(19) United States
(12) Patent Application Publication
(10) Pub. No.: US 2024/0382624 A1
(43) Pub. Date: Nov. 21, 2024

(54) FIBROBLAST ACTIVATION PROTEIN TARGETED DYES THEIR RELATED USES

(71) Applicant: On Target Laboratories, Inc., West Lafayette, IN (US)

(72) Inventors: Sumith A Kularatne, West Lafayette, IN (US); Pravin Gagare, West Lafayette, IN (US); Mini Thomas, West Lafayette, IN (US)

(21) Appl. No.: 18/660,867

(22) Filed: May 10, 2024

Related U.S. Application Data
(63) Continuation of application No. 18/655,248, filed on May 4, 2024.
Application No. 18/655,248 is a continuation-in-part of application No. PCT/US2022/048858, filed on Nov. 3, 2022.
(60) Provisional application No. 63/276,157, filed on Nov. 5, 2021.

(51) Int. Cl. A61K 49/00

(52) U.S. Cl. CPC A61K 49/0032; A61K 49/0056

(57) ABSTRACT

The present disclosure relates to Fibroblast Activating Protein (FAP) targeted compounds conjugated to near-infra red (NIR) dyes and methods for their therapeutic and diagnostic use. More specifically, this disclosure provides compounds and methods for diagnosing and treating diseases associated with cells and/or vasculature expressing Fibroblast Activating Protein (FAP). The disclosure further describes methods and compositions for making and using the compounds, methods incorporating the compounds, and kits incorporating the compounds.

### RELATED APPLICATIONS

The present patent application is a continuation of U.S. patent application Ser. No. 18/655,248, filed May 4, 2024, which is a continuation in part of International Patent Application No. PCT/US22/48858, filed Nov. 3, 2022 which claims the priority benefit of U.S. Provisional Patent Application Ser. No. 63/276,157, filed Nov. 5, 2021, the content of each is hereby incorporated by reference in its entirety into this disclosure.

### BACKGROUND

Fibroblast activation protein (FAP) is a serine protease with post-proline dipeptidyl peptidase and endopeptidase enzymatic activity. FAP is also known as Surface Expressed Protease (seprase), antiplasmin-cleaving enzyme (APCE), (EC3.4.2.1.B28)). FAP is upregulated in several tumor types, while its expression in healthy adult tissues is scarce. The FAP molecule itself and FAP⁺ stromal cells play an important, although probably context-dependent, and tumor type-specific pathogenetic role in tumor progression. FAP is characteristically expressed by various cell types in the microenvironment of human malignancies, and which may be a promising therapeutic target in cancer treatment. FAP also express in non-malignant conditions, including myocardial infarction, liver cirrhosis, pulmonary fibrosis, osteoarthritis, and rheumatoid arthritis.

Imaging agents, such as optical imaging agents, are a valuable tool for image-guided surgery. Image-guided surgery helps surgeons perform safer and less invasive procedures and has become a recognized standard of care in managing disorders including cranial, otorhinolaryngology, spine, orthopedic, and cardiovascular. However, a need exists for an suitable FAP-imaging agents for the specific targeting of tumors for imaging, diagnostic, or therapeutic purposes, or for laboratory purposes in the study of endogenous FAP expression.

### BRIEF SUMMARY

The present disclosure relates to imaging agents conjugated to fibroblast activating protein (FAP) targeting compounds and methods for their imaging, therapeutic, or diagnostic use. More specifically, this disclosure provides compounds and methods for diagnosing and surgical removal (image-guided surgery) of cells and/or micro-environment expressing FAP, such as cancer of colon, kidney, endometrial, urinary, colorectal, ovarian, breast, pancreatic, prostate, liver, and esophagus and related diseases. The disclosure further describes methods and compositions for making and using the compounds, methods incorporating the compounds, and kits incorporating the compounds.

One aspect of the disclosure includes a compound having a formula B—X—Z, wherein B comprises a Fibroblast Activating Protein (FAP)-targeting molecule or FAP inhibitor (FAPI); X comprises a spacer; and N comprises an imaging agent.

In an aspect, B is a monovalent group derived from a FAP-targeted molecule.

In an aspect, B is a small molecule comprising an extended hydrophobic moiety with alpha keto amide unit, a substituted alpha keto amide, or a substituted alpha keto amide with aromatic moiety with methoxy units.

In an aspect, B has a formula:

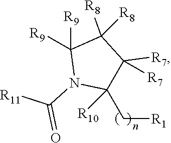

wherein R¹ is CHO, C═CH, CH(CN)₂, CN, NHCN, B(OH)R₂, COR₂, Zn(OH), PO(OH)R₂, SO₂R₂, Si(OH)₂R₂, a halogen, OH, H or COCONHR₃;
wherein R₂ is OH, SH, NH₂, O-alkyl, S-alkyl, NH-alkyl, a halogen, or H; and
R₃ is H, a halogen, OH, methyl, ethyls, isopropyl, tBu, cyclopropyl, alkyl, alkyl-OH, CN, CHPh₂, adamantane or

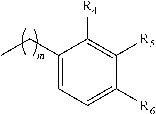

wherein R₄, R₅, or R₆ are independently selected from the group consisting of H, OCH₃, SCH₃, COR₂, SO₂R₂, PO(OH)R₂, Si(OH)₂R₂, halogen, OH, CN, B(OH)R₂, and combinations thereof;
R₇, R₈, R₉, or R₁₀ are independently selected from the group consisting of H, a halogen, OH, OCH₃, SCH₃, CN, COR₂, SO₂R₂, PO(OH)R₂, Si(OH)₂R₂, B(OH)R₂, a C₁-C₁₀ alkyl, and combinations thereof;
n and m are independently selected from 0, 1, 2, 3, 4, or 5;
R₁₁ is selected from the group consisting of

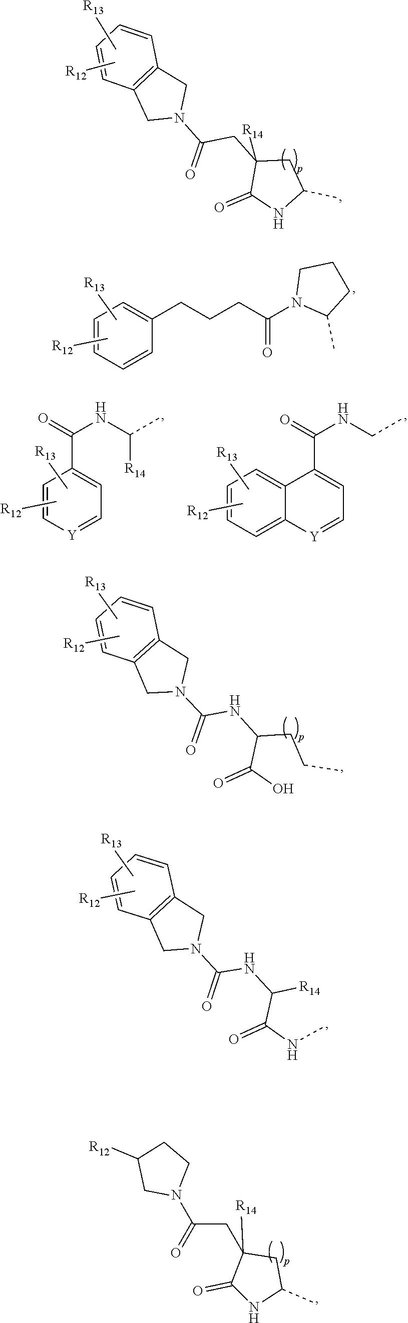

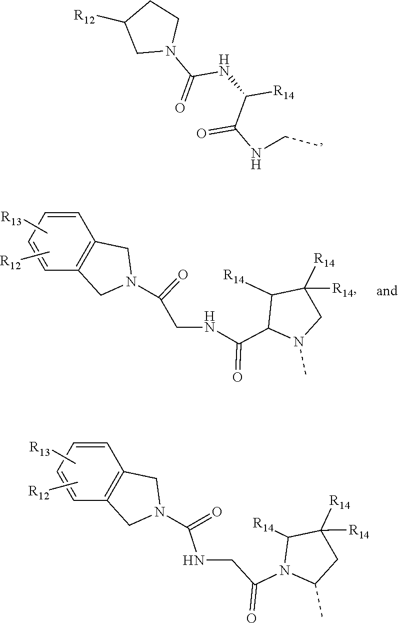

wherein R₁₂ is (CH₂)q-X, X═COOH, SH, OH, NH₂, halogen, or maleimide;
R₁₃ is H, halogen, Me, OH, SH, OCH₃, OBn, or CH₂OH;
R₁₄ is H, F, CH₃, OCH₃, Isopropyl, or Tert-butyl;
Y is CH or N; and
p and q are independently selected from 0, 1, 2, 3, 4, or 5.

In an aspect, X is selected from the group consisting of six aminohectanoic acid (SAHA), eight aminooctonoic acid (EAOA), polyethylene glycol (PEG), polyethylene amine (PEA) unit, and N-amino-dPEG₂ acid.

In an aspect, X is a peptide comprising at least one aryl group or at least one aryl alkyl group, each of which is optionally substituted.

In an aspect, the peptide comprises at least two aryl or aryl alkyl group, wherein a first aryl or aryl alkyl group is about 6 atoms to about 15 atoms.

In an aspect, the peptide compromises at least one amino acid selected from the group consisting of a quaternary amine containing amino acid, an acidic amino acid, a basic amino acid, a neutral polar amino acid, a neutral nonpolar amino acid, an aromatic amino acid, and amino acid derivatives.

In an aspect, the acidic amino acid is selected from the group consisting of aspartic acid and glutamic acid; and/or the basic amino acid is selected from the group consisting of arginine, lysine, histidine, and ornithine; and/or the neutral polar amino acid is selected from the group consisting of glycine, serine, threonine, cysteine, tyrosine, asparagine, and glutamine; and/or the neutral nonpolar amino acid is selected from the group consisting of alanine, leucine, isoleucine, valine, proline, phenylalanine, tryptophan, and methionine; and/or amino acid derivative is derived from tyrosine.

In an aspect, the amino acid derivative is selected from the group consisting of:

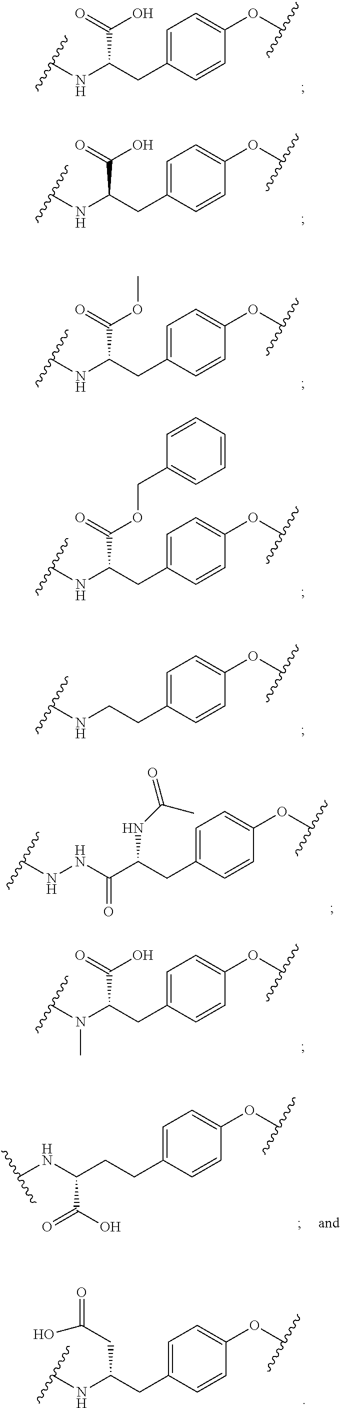

In an aspect, X comprises an amino acid spacer with sulfur-containing side chain group, wherein the sulfur-containing side group is selected from the group consisting of cysteine, methionine and a molecule containing a thiophenol moiety or an amino acid spacer with a chalcogen-containing side chain group.

In an aspect, X comprises a single amino acid selected from the group consisting of tyrosine, cysteine, lysine, tyramine, a tyrosine derivative, a cysteine derivative, and a lysine derivative.

In an aspect, X comprises an amino acid isotope.

In an aspect, X has a length of about 1 atoms to about 20 atoms.

In an aspect, Z comprises a monovalent group derived from a NIR dye.

In an aspect, the NIR dye is selected from the group consisting of:

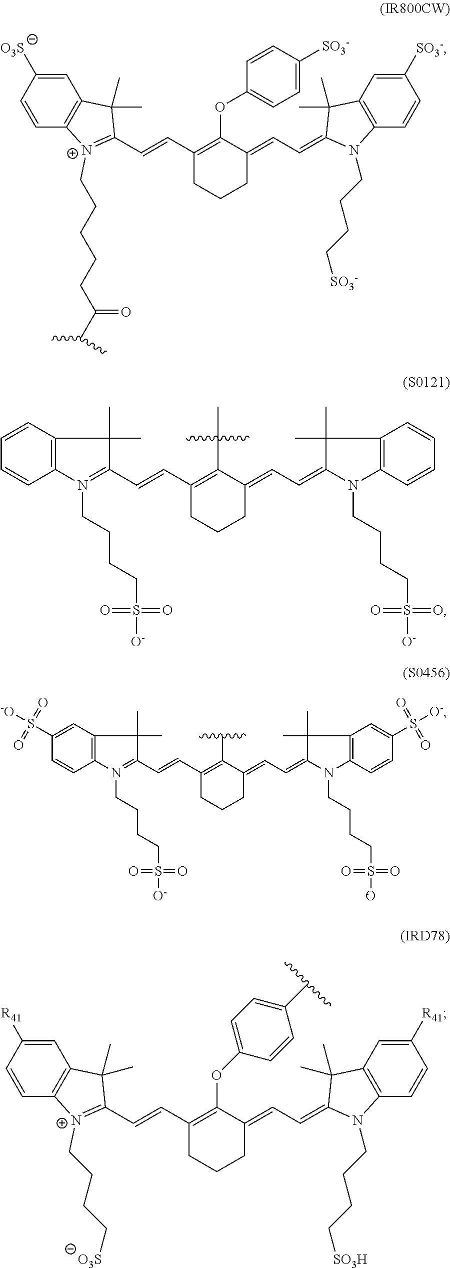

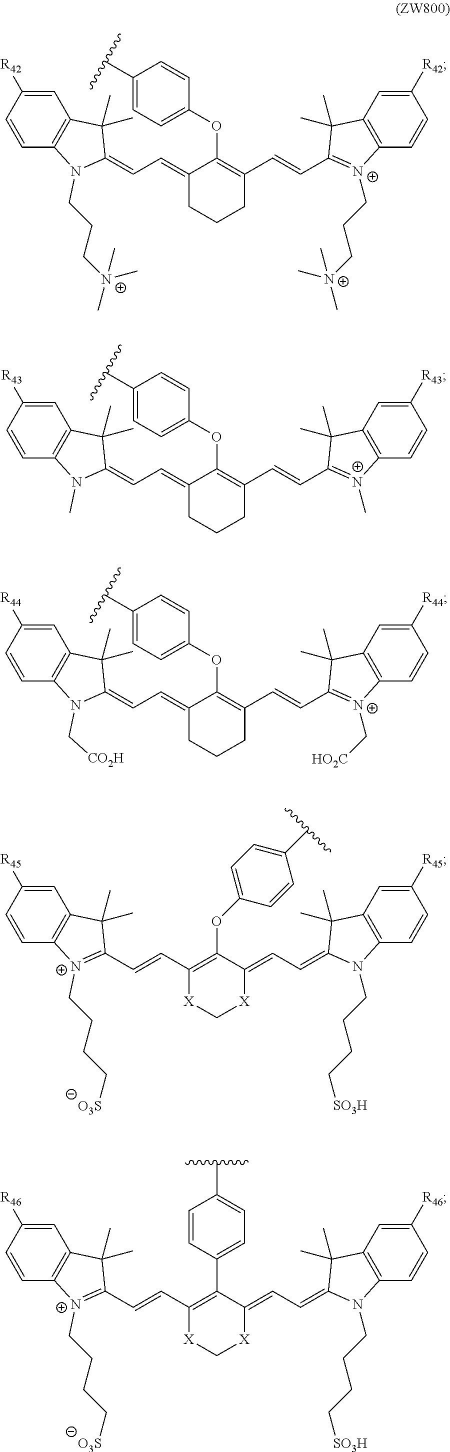

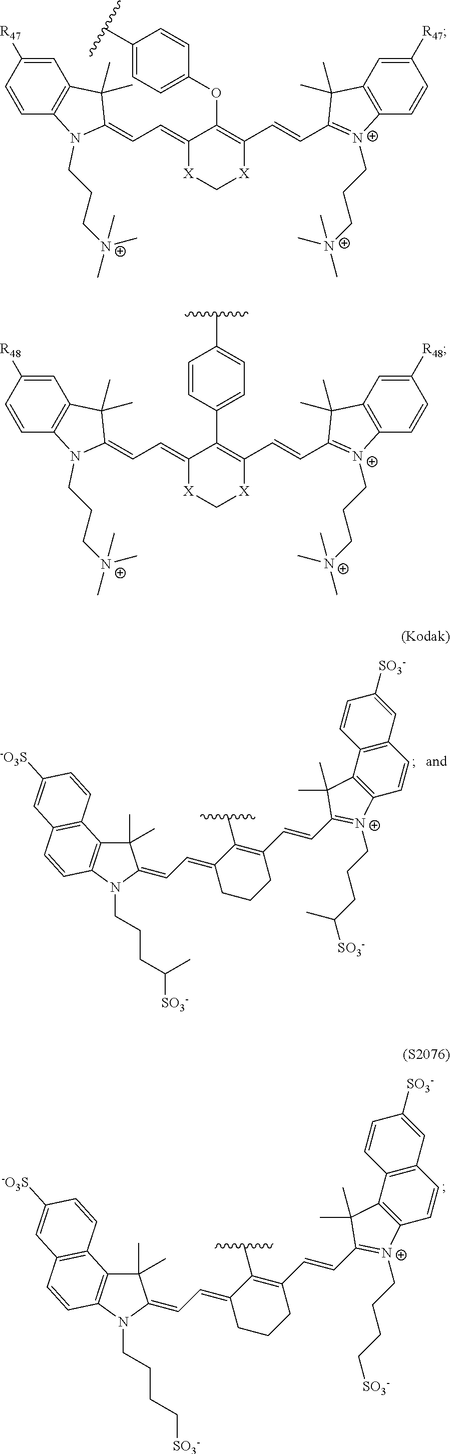

wherein R41, R42, R43, R44, R45, R46, R47, R48=H or SO₃H; X═O, S, or N.

One aspect of the disclosure is a compound selected from the group consisting of:

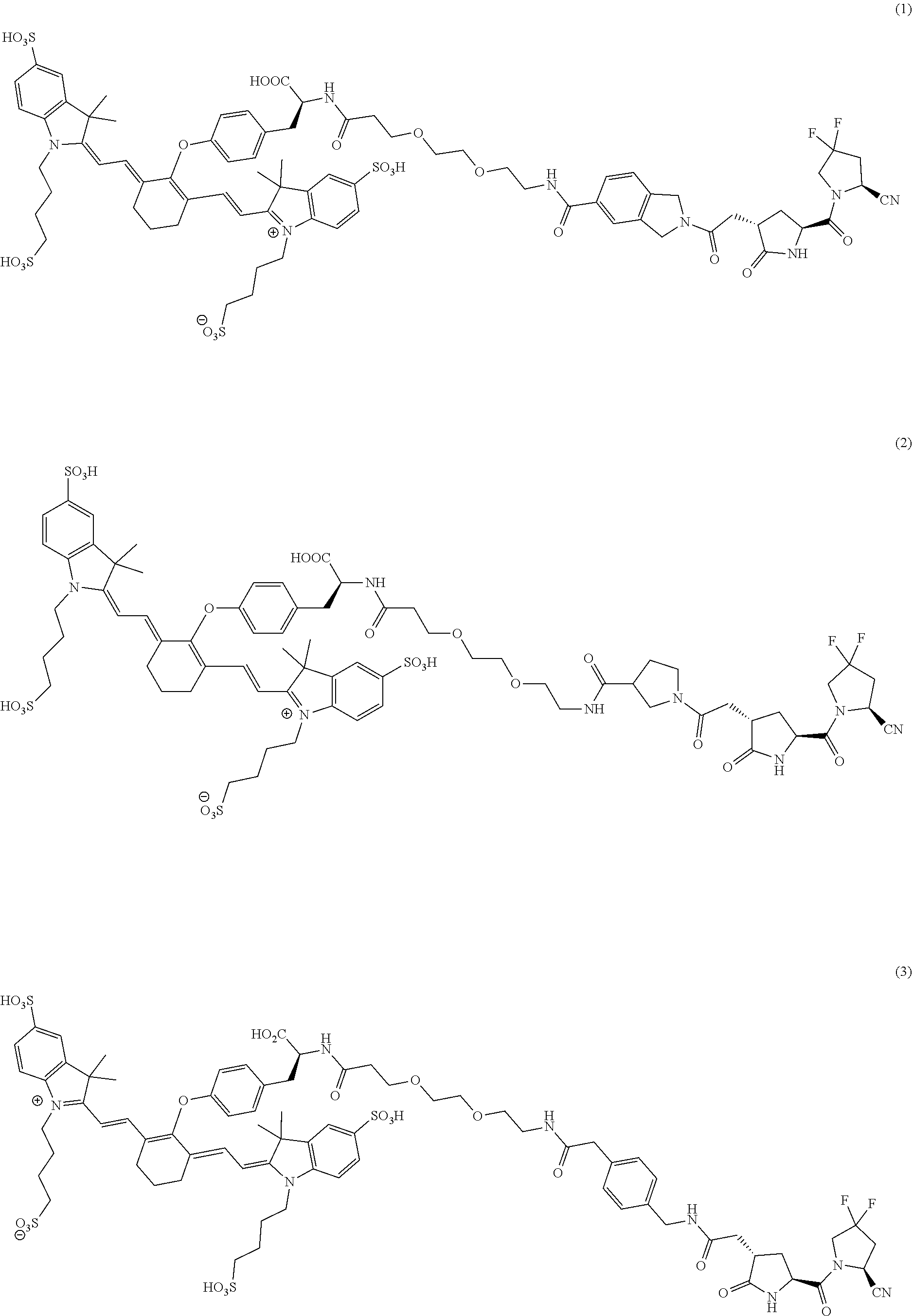

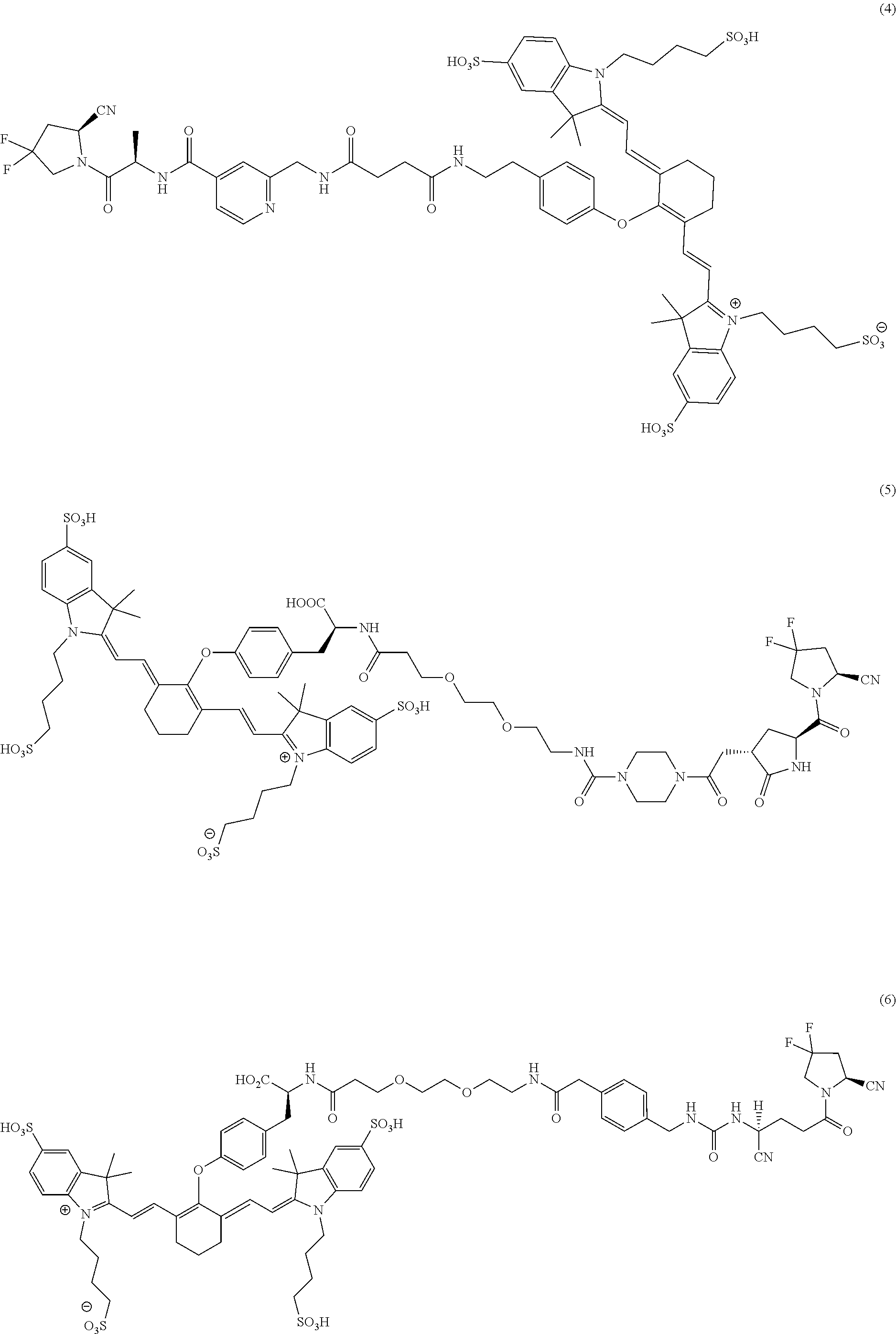

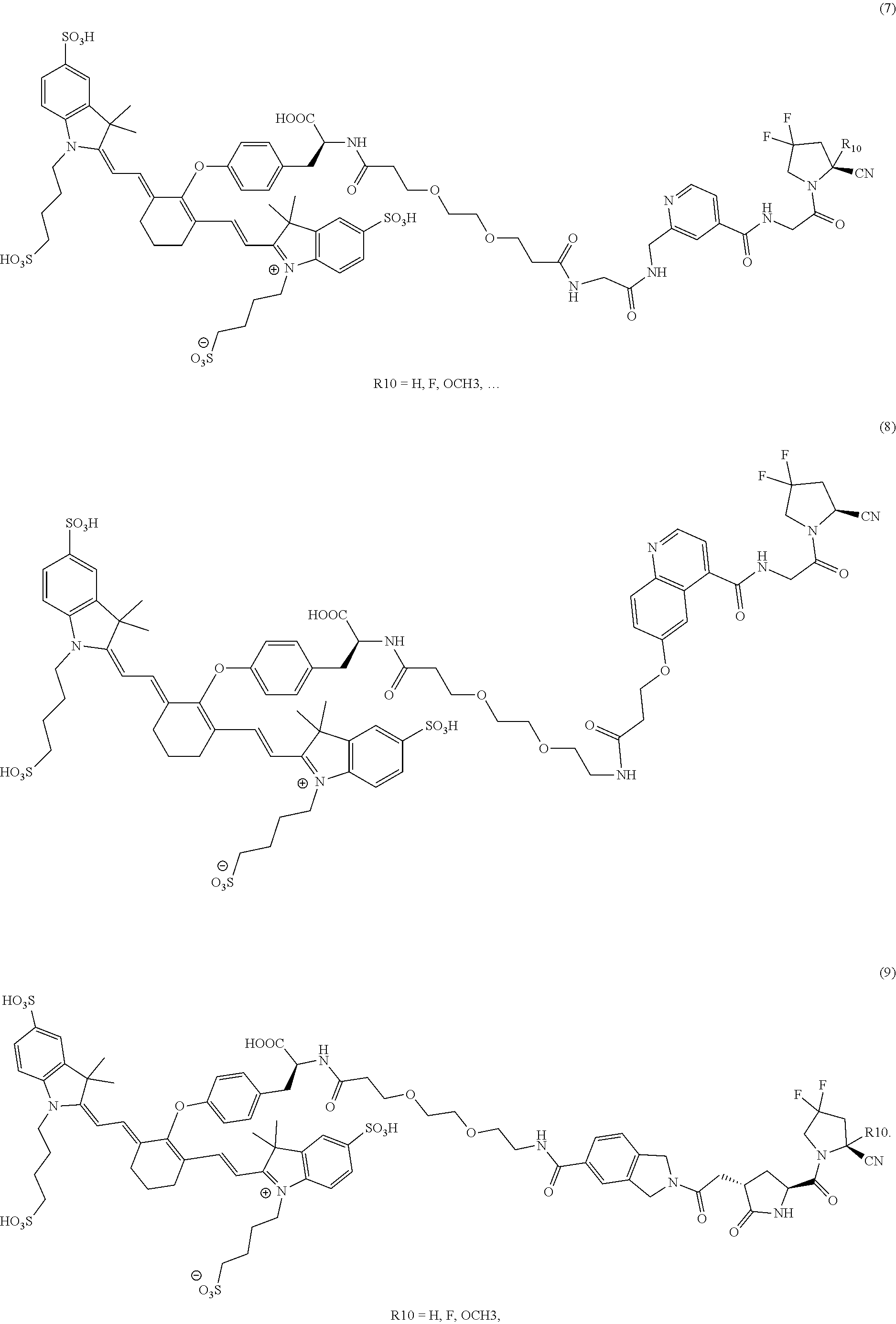

One aspect of the disclosure is a selected from the group consisting of:

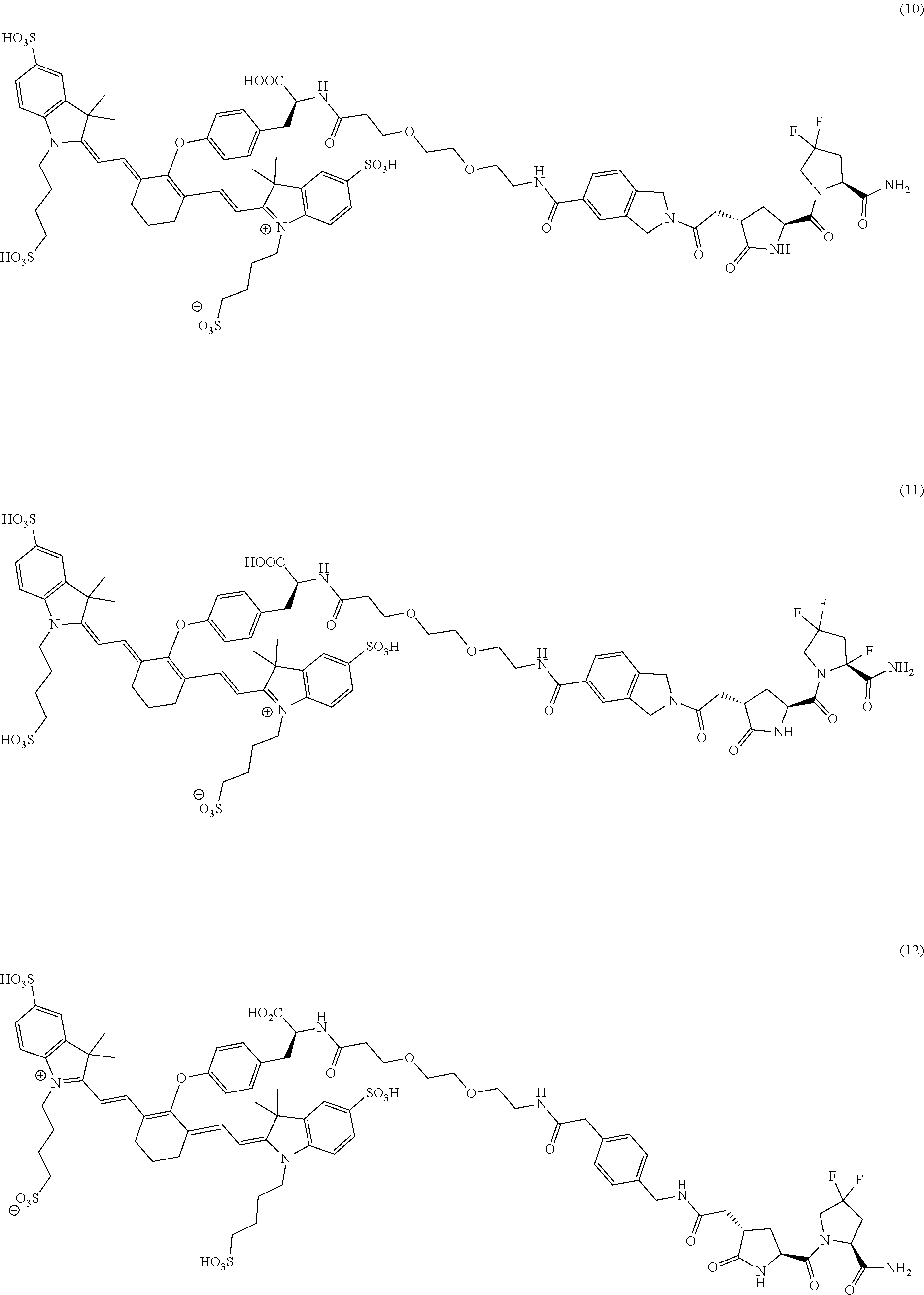

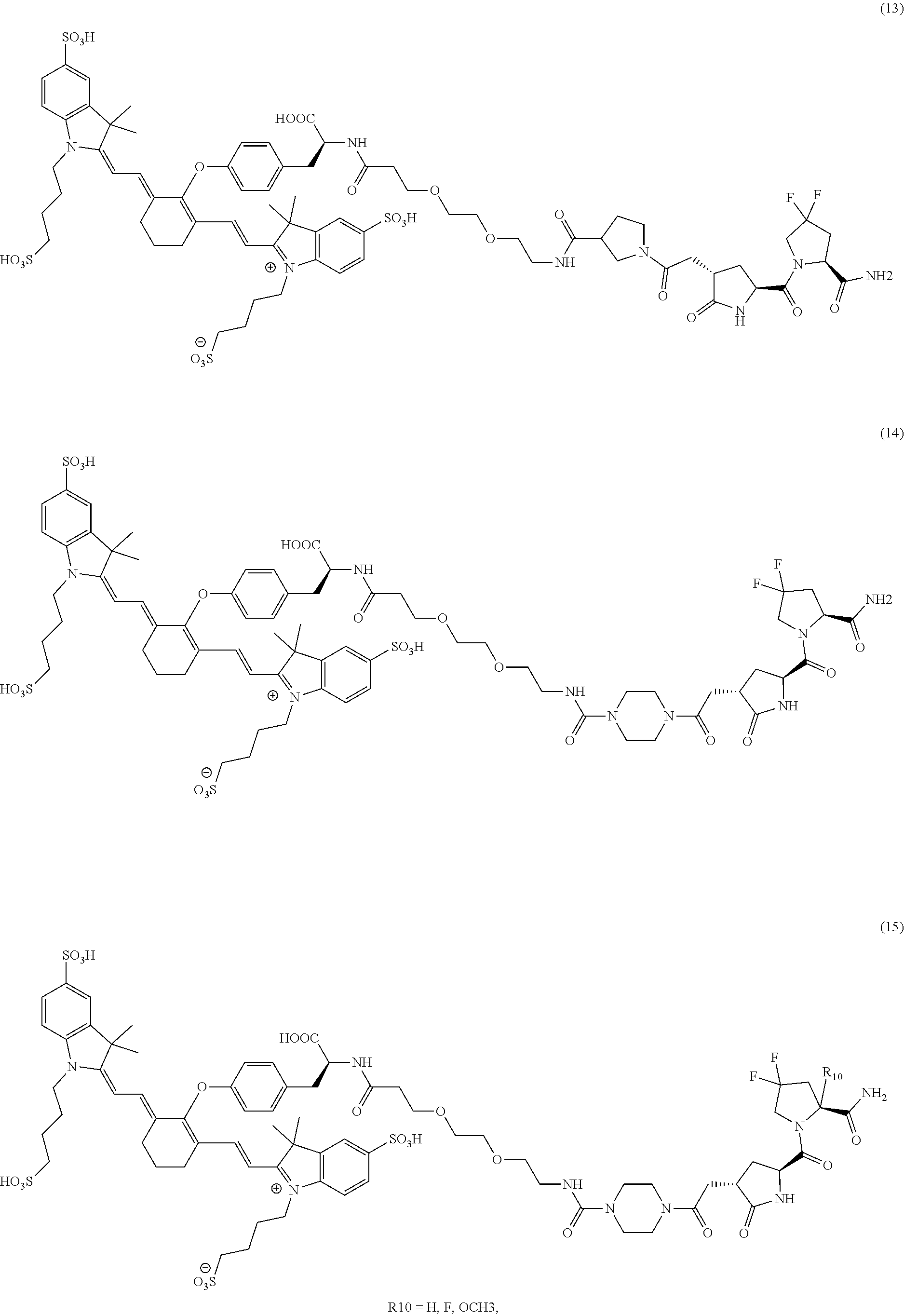

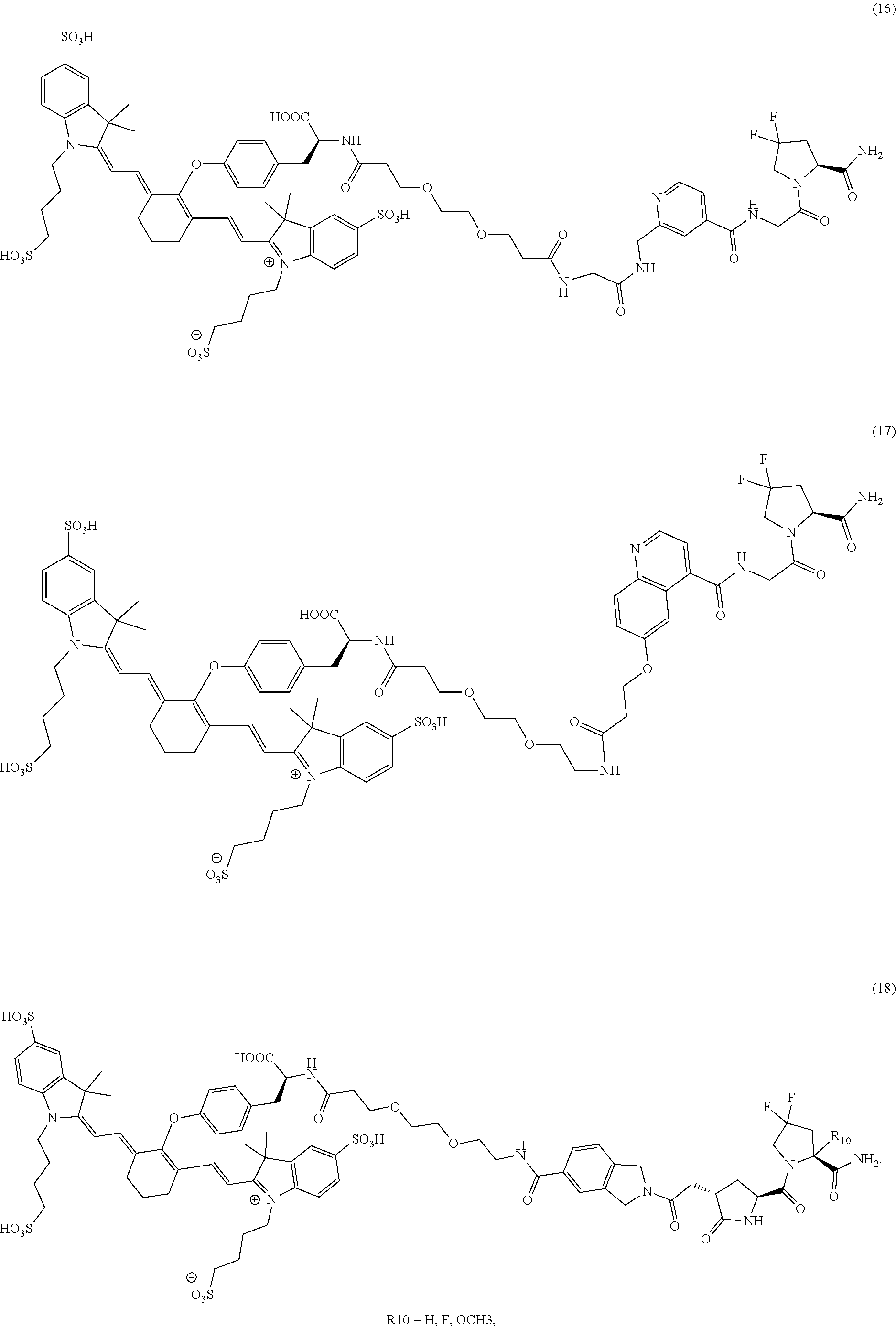

One aspect of the disclosure is a compound selected from the group consisting of:

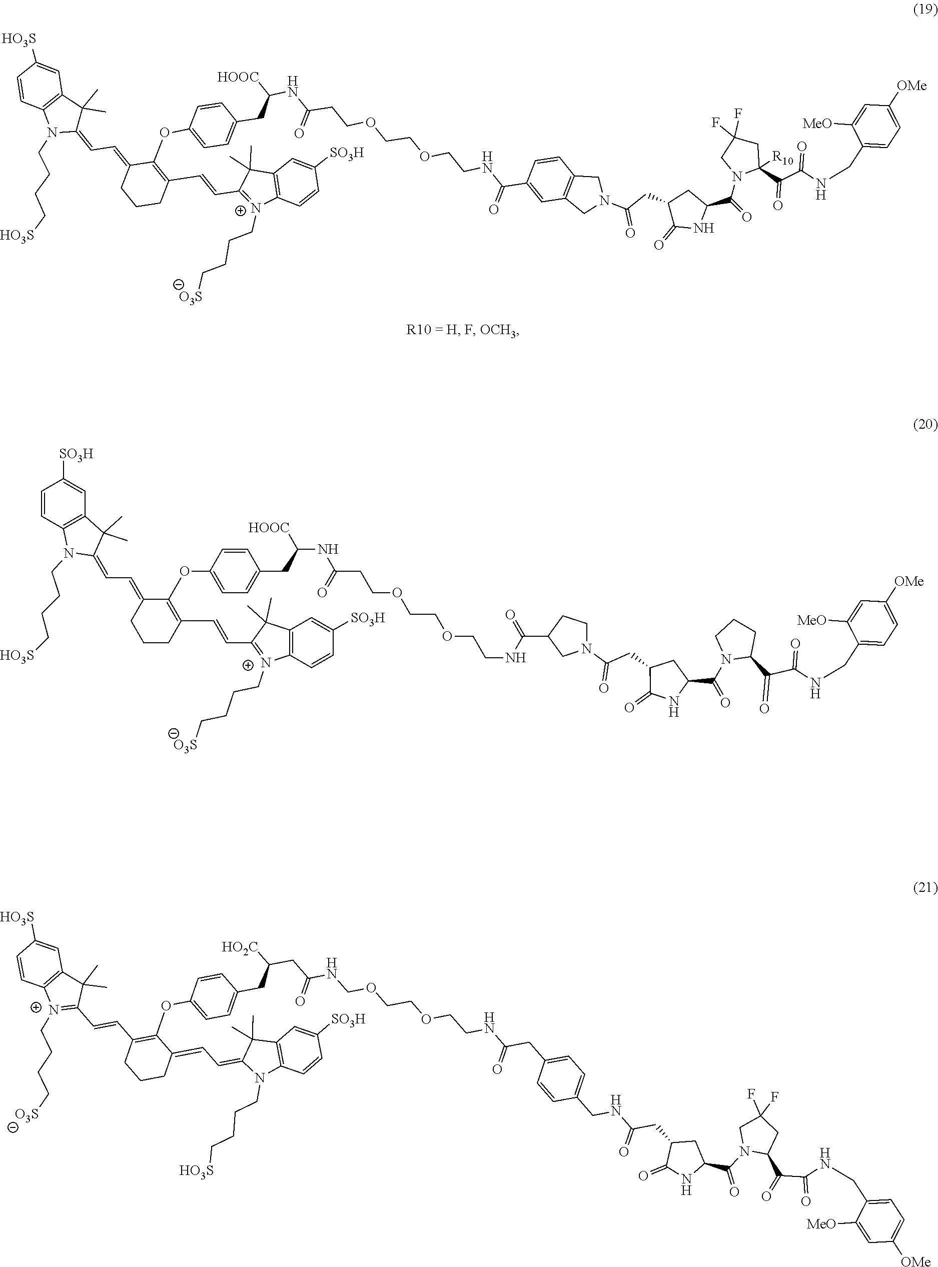

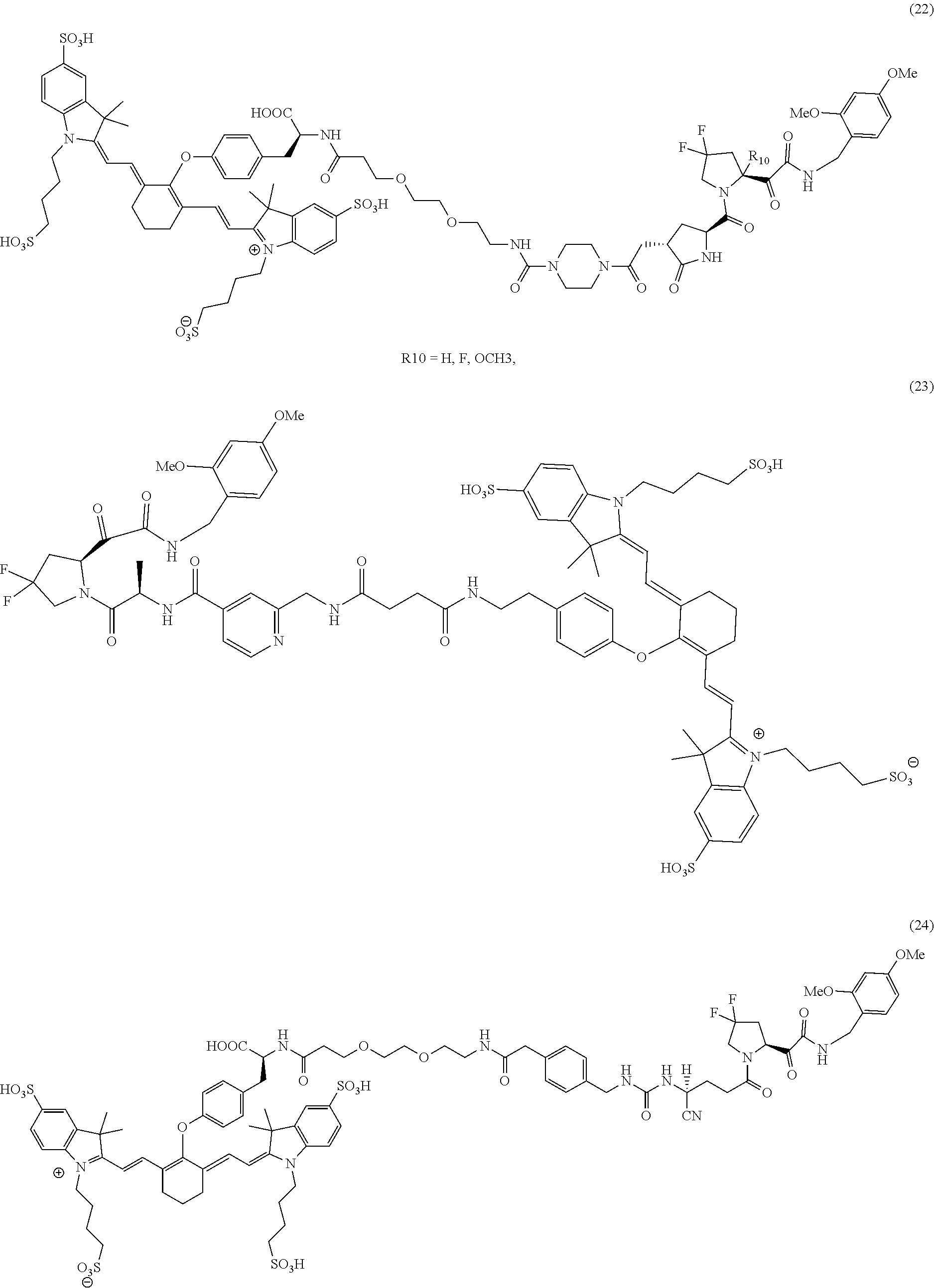

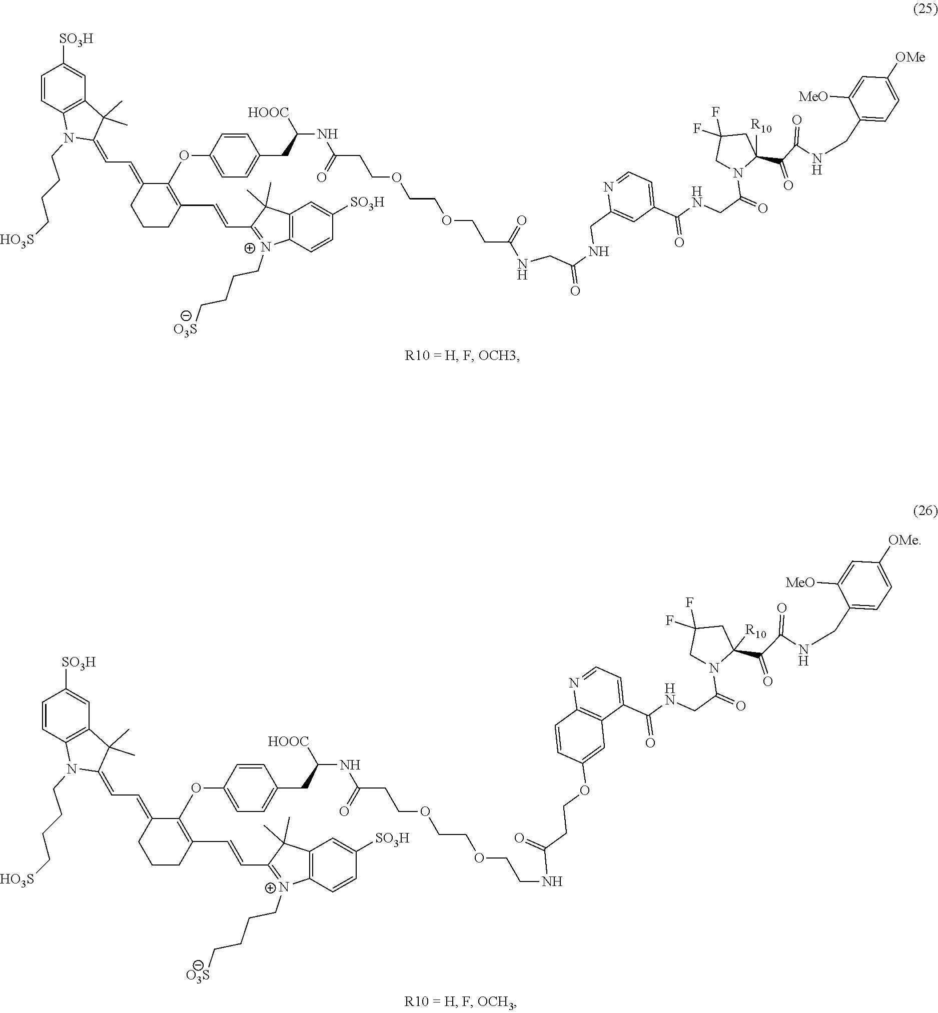

One aspect of the disclosure is a compound selected from the group consisting of:

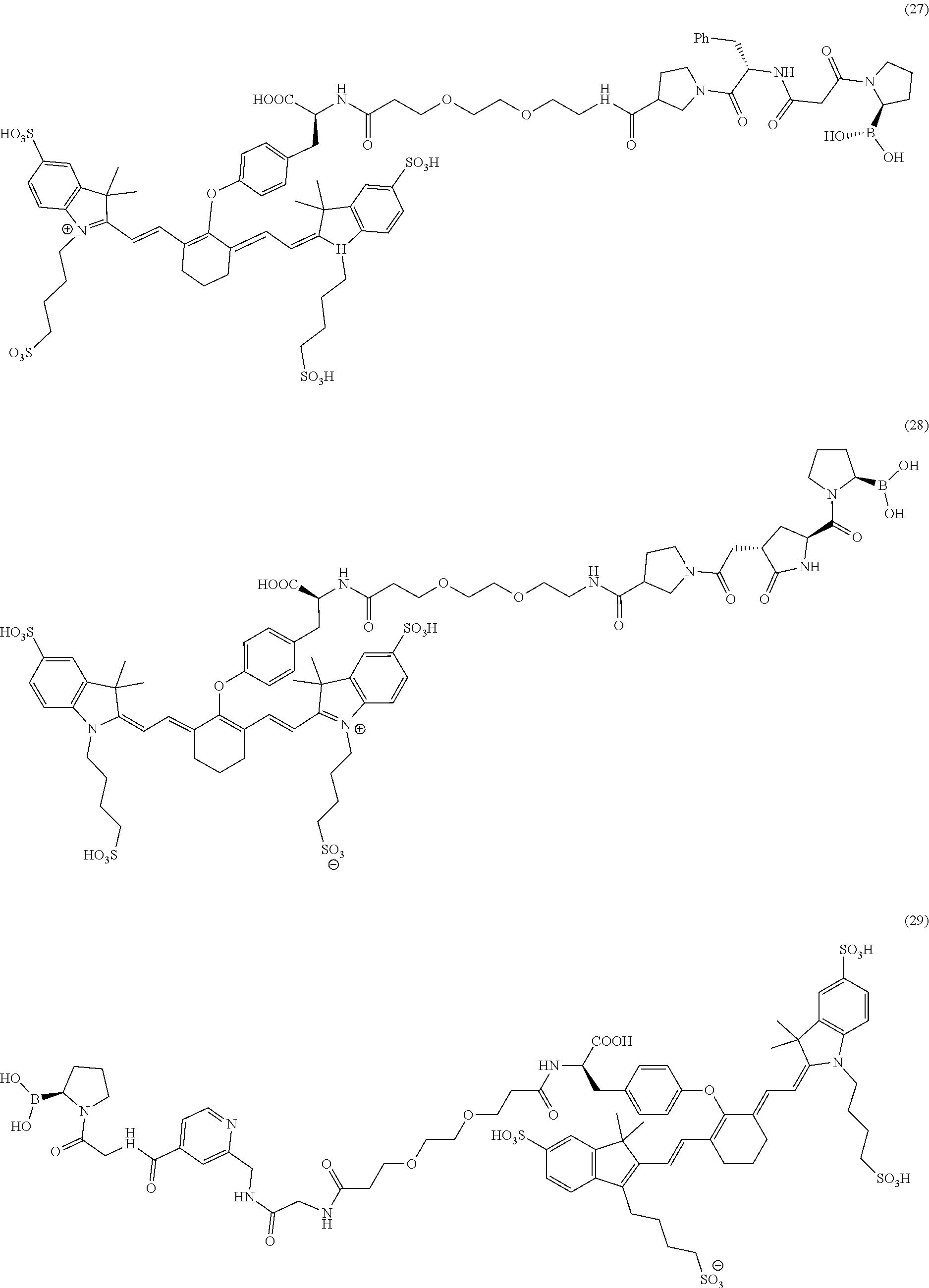

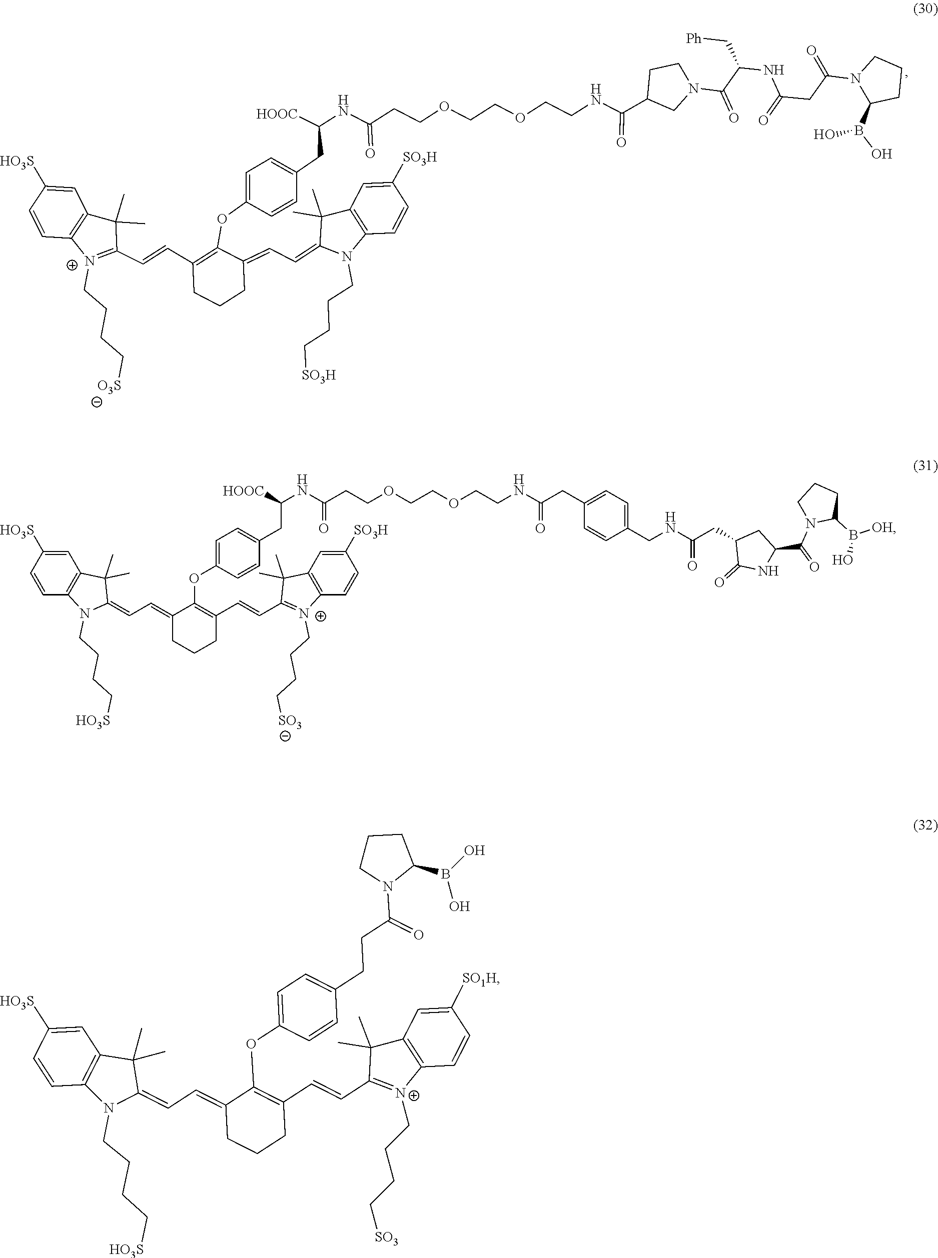

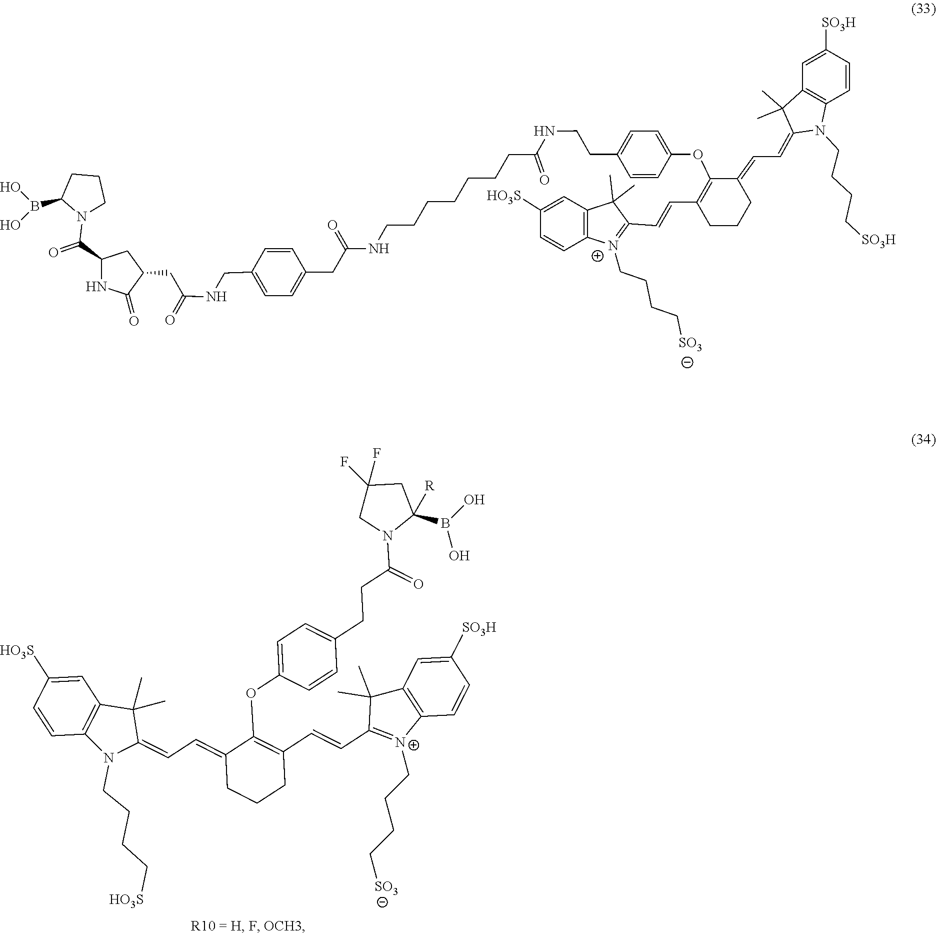

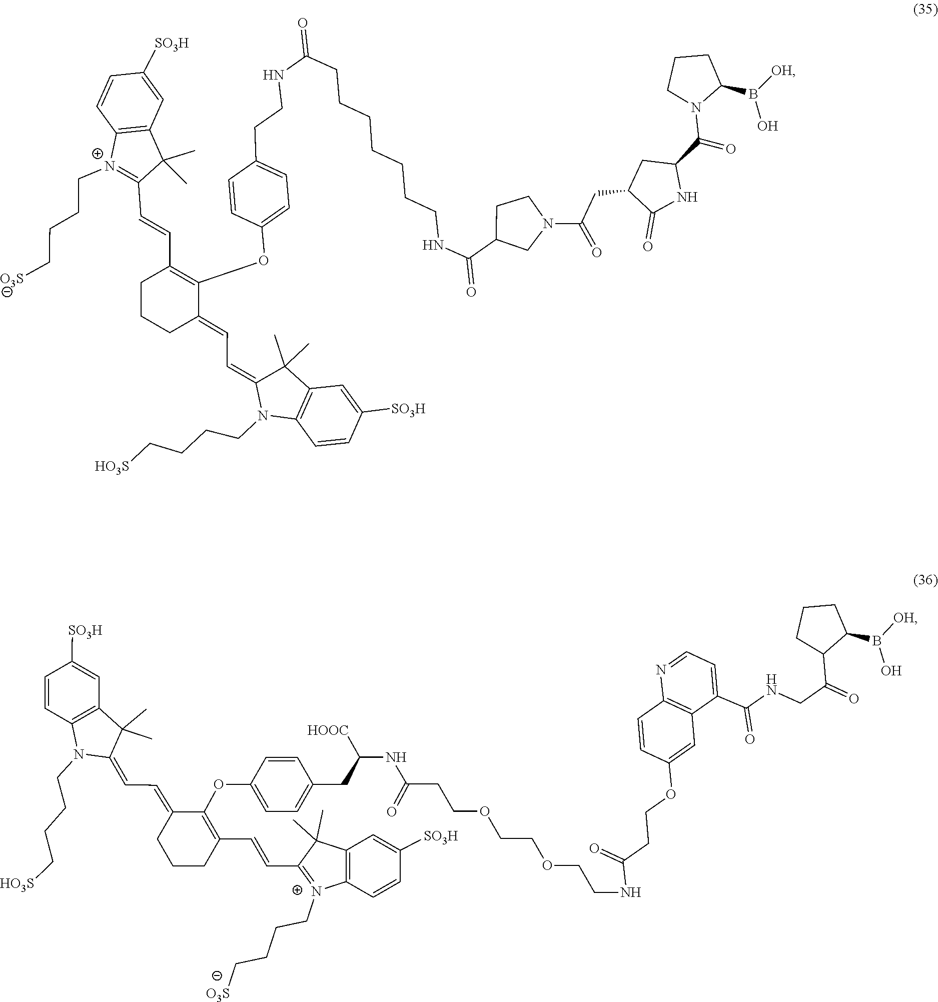

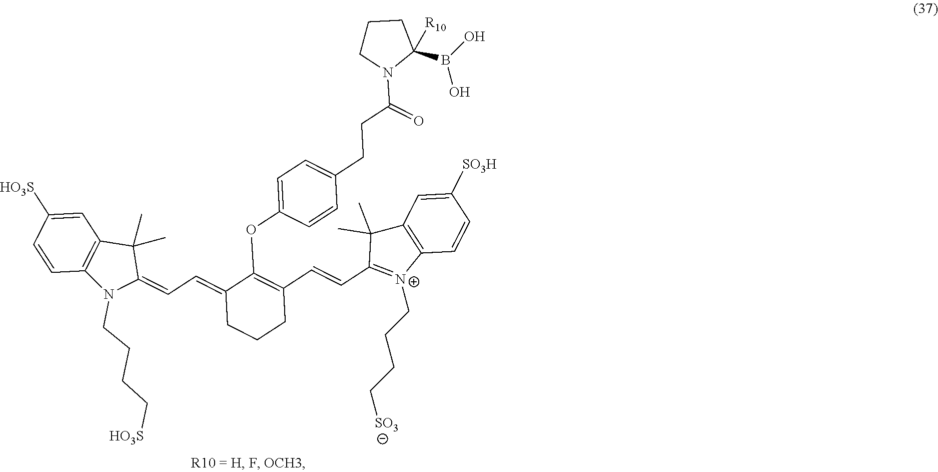

One aspect of the disclosure is a compound selected from the group consisting of:

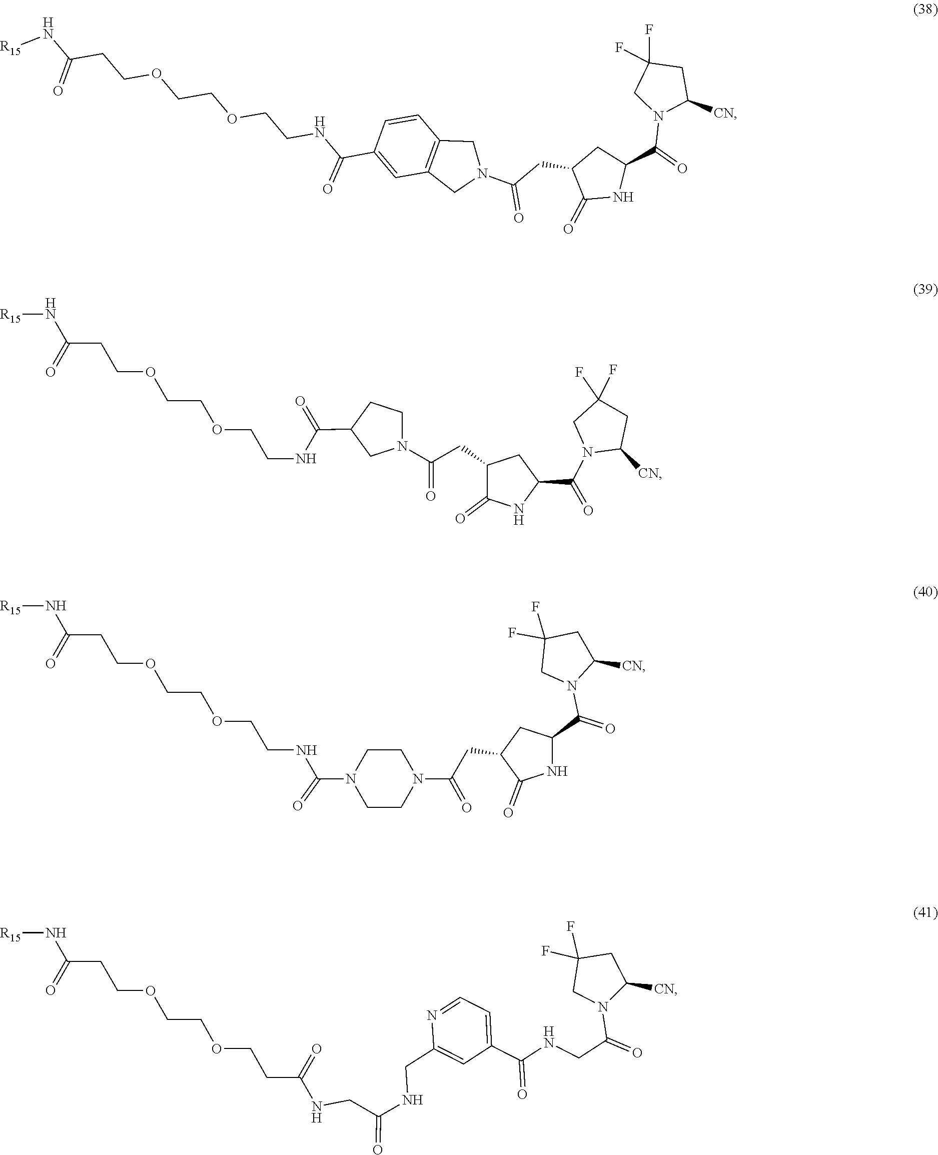

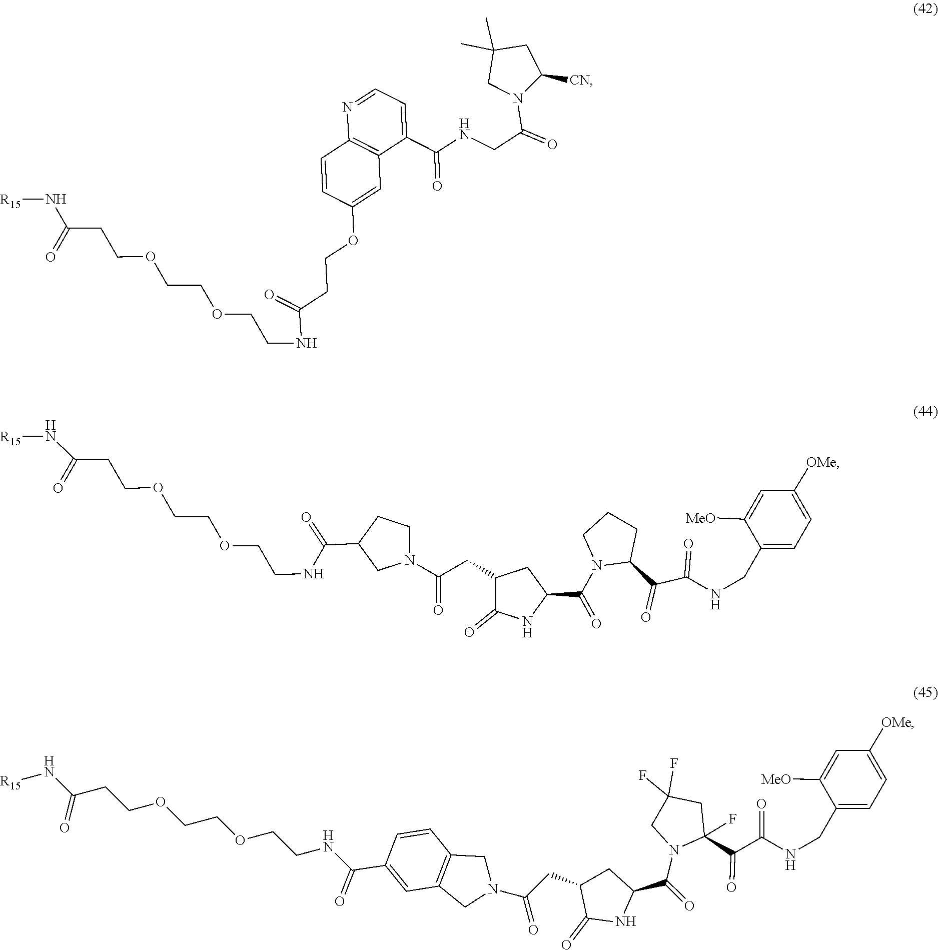

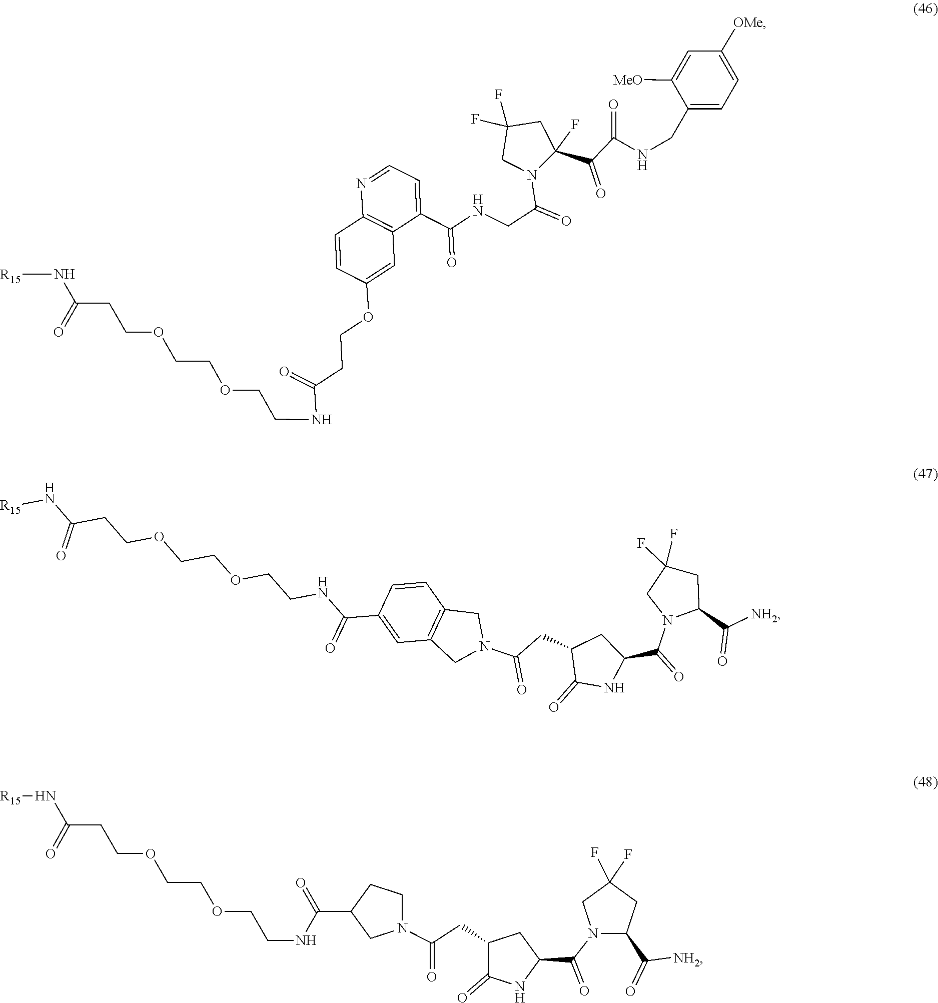

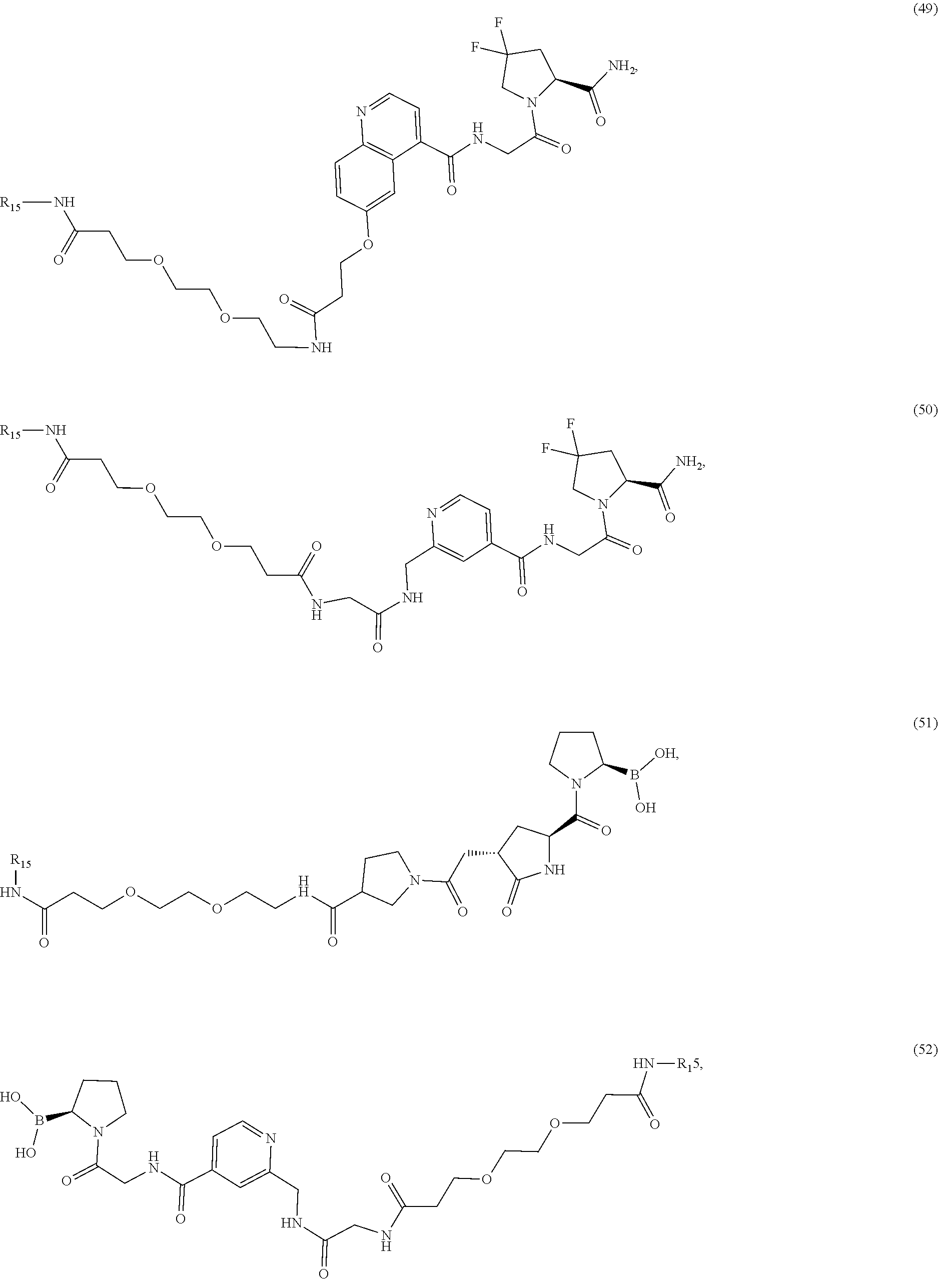

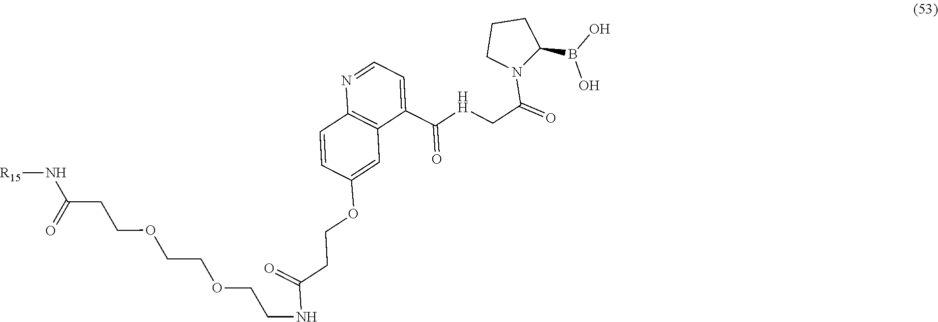

wherein R₁₅ is chelating group selected from the group consisting of:

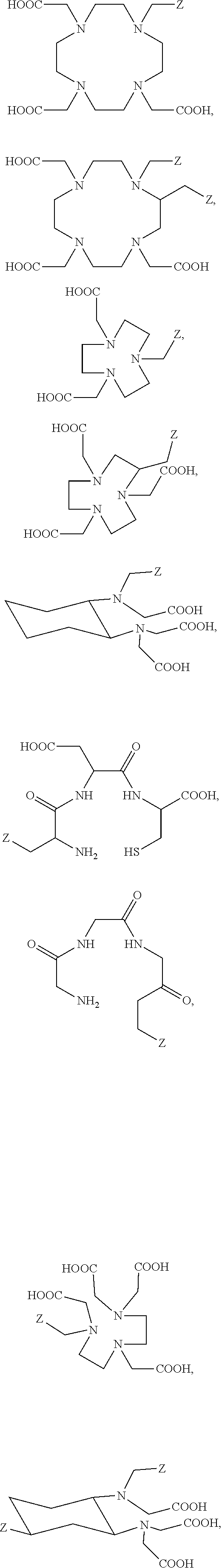

and Z═CO—, CH₂-Ph-NH—CO—, CH(COOH)—(CH₂)₂—CO—.

### BRIEF DESCRIPTION OF THE DRAWINGS

Various aspects of the present disclosure will now be described, by way of example only, with reference to the attached Figures, wherein:

FIGS. 1A-1C show the chemical structure of Fibroblast Activating Protein Inhibitors with Cyanide (FAPI-Cyno) functional group-NIR dye conjugates for use in Image-Guided Surgery (IGS)

FIGS. 2A-2C show the chemical structure of Fibroblast Activating Protein Inhibitors with Amide (FAPI-Amido) functional group-NIR dye conjugates for use in IGS.

FIGS. 3A-3C show chemical structure of Fibroblast Activating Protein Inhibitors with α-Keto Amide (FAPI-KetoAmido) functional group-NIR dye conjugates for use in IGS.

FIGS. 4A-4D show chemical structure of Fibroblast Activating Protein Inhibitors with Boronic acid (FAPI-Boro) functional group-NIR dye conjugates for use in IGS.

FIGS. 5A-5H shows the chemical structure of Fibroblast Activating Protein Inhibitors with cyno, amido, ketoamido, or Boro functional groups-radio conjugates Image-Guided Surgery (IGS)/Radio-guided surgery (RGS) conjugates. The chelating group is chelated with metal such as 64Cu, 67Cu, 68Ga, 89Zr, 90Y, 99mTe, 111 In, 153Sm, 177Lu, 186Re, 188Re, 223Ra or 225Ac and use with tomographic methods to guide the surgery.

FIG. 6. shows a LCMS Chromatogram of trans-55.

FIG. 7. shows a LCMS Chromatogram of trans-56.

FIG. 8. shows a LCMS Chromatogram of trans-58.

FIG. 9. shows a LCMS Chromatogram of trans-59.

FIG. 10. shows a LCMS Chromatogram of compound 72.

FIG. 11. shows a LCMS Chromatogram of compound 79.

FIG. 12. shows a LCMS Chromatogram of 3-(2-(2-(2-(2-((3S,5S)-5-((S)-2-cyano-4,4-difluoropyrrolidine-1-carbonyl)-2-oxopyrrolidin-3-yl) acetyl) isoindoline-5-carboxamido)ethoxy)ethoxy)propanoyl)-L-tyrosine.

FIG. 13. shows a LCMS Chromatogram of compound 1.

FIG. 14. shows a LCMS Chromatogram of compound 10.

FIG. 15. shows a LCMS Chromatogram of compound 81.

FIG. 16 shows a LCMS Chromatogram of (3-(2-(2-(2-(4-((2-((3S,5S)-5-((S)-2-cyano-4,4-difluoropyrrolidine-1-carbonyl)-2-oxopyrrolidin-3-yl) acetamido)methyl)phenyl) acetamido) ethoxy) ethoxy) propanoyl)-L-tyrosine.

FIG. 17 shows a LCMS Chromatogram of compound 3.

FIG. 18 shows a LCMS Chromatogram of (3-(2-(2-(1-(2-((3S,5S)-5-((S)-2-cyano-4,4-difluoropyrrolidine-1-carbonyl)-2-oxopyrrolidin-3-yl) acetyl) pyrrolidine-3-carboxamido)ethoxy)ethoxy)propanoyl)-L-tyrosine.

FIG. 19 shows a LCMS Chromatogram of compound 2.

FIG. 20 shows a LCMS Chromatogram of compound 85.

FIG. 21 shows a LCMS Chromatogram of (3-(2-(2-(4-(2-((3S,5S)-5-((S)-2-cyano-4,4-difluoropyrrolidine-1-carbonyl)-2-oxopyrrolidin-3-yl)acetyl)piperazine-1-carboxamido)ethoxy)ethoxy)propanoyl)-L-tyrosine.

FIG. 22 shows a LCMS Chromatogram of compound 5.

FIG. 23 shows a LCMS Chromatogram of compound 6.

FIG. 24 shows an overlay of whole-body fluorescence image of over white light images after adjusting the threshold U87MG human Malignant Glioma tumor xenograft bearing mouse who were administered a compound according to an aspect of the disclosure.

FIG. 25 shows an overlay of whole-body fluorescence images over white light images after adjusting the threshold of HT human colorectal tumor xenograft bearing mice who were administered a compound according to an aspect of the disclosure.

### DETAILED DESCRIPTION

This disclosure provides FAP-targeted ligands linked to imaging agents via different linkers to improve clinical properties (e.g., stability, PK properties, solubility, fast tumor accumulation, higher fluorescence, fast skin clearance, and higher tumor-to-background ratios) of the compounds. The disclosure provides uses of the compounds in image-guided surgery and methods for synthesizing the same. This disclosure also provides novel higher affinity ligands to improve in vivo affinity and PK properties of, for example, NIR conjugates. This disclosure also provides compounds for use in the targeted imaging of tumors expressing FAP, including but not limited to cancer, and methods of use, for example, in imaging and surgery involving FAP positive tissues and tumors.

### I. Definitions

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The singular form “a”, “an” and “the” include plural referents unless the context clearly dictates otherwise. These articles refer to one or to more than one (i.e., to at least one). As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. Herein, “up to” a number (for example, up to 50) includes the number (for example, 50). The term “in the range” or “within a range” (and similar statements) includes the endpoints of the stated range.

Reference throughout this specification to “one aspect,” “an aspect,” “certain aspects,” or “some aspects,” etc., means that a particular feature, configuration, composition, or characteristic described in connection with the aspect is included in at least one aspect of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

The invention is defined in the claims. However, below is a non-exhaustive listing of non-limiting exemplary aspects. Any one or more of the features of these aspects may be combined with any one or more features of another example, embodiment, or aspect described herein.

### II. Molecular Imaging and Imaging Agents

Molecular imaging is an important tool for in vivo cancer visualization. The use of molecular imaging may improve the diagnostic performance for early cancer detection, tumor staging, risk stratification, and guidance of therapy. Various imaging agents may be used with molecular imaging techniques. In some embodiments, the molecular imaging is computed tomography (CT), magnetic resonance imaging (MRI), and ultrasound imaging (UI) and the corresponding imaging agent is used. However, while these approaches may be effective for diagnosing cancer, its low resolution makes it challenging to detect early-stage cancer, and accurately removing the lesion during surgery can lead to numerous side effects.

In some aspects, nuclear techniques like positron emission tomography (PET) and single-photon emission computed tomography (SPECT) may be employed to visualize and quantify physiological processes through the use of radiotracers. For instance, in clinical settings, 2-deoxy-2-18F-fluoro-D-glucose (18FDG) is regularly utilized alongside PET for cancer staging. In some embodiments, the nuclear techniques may be used for radio-guided surgery. In some aspects, the radio-guided surgery may employ PET imaging. In other aspects, the radio-guided surgery may employ SPECT imaging. In a non-limiting aspect, the SPECT imaging employes the use of radioisotopes such as iodine-123, technetium-99m, xenon-133, thallium-201, and indium-111.

In some embodiments, optical imaging techniques are employed. In comparison to other cancer imaging techniques, optical imaging offers high resolution for observing real-time functional and structural changes. It is an in vivo, noninvasive method that may protect patients from the potential risks associated with non-optical methods like biopsy and subsequent histologic examination. In some embodiments, the optical imaging technique is confocal imaging. In some embodiments, the optical imaging technique is optical molecular imaging. In some embodiments, the optical imaging technique is optical coherence tomography (OCT). In some embodiments, the optical imaging technique is fluorescence imaging (FI). In some embodiments, the optical imaging technique is near-infrared (NIR) fluorescence imaging.

In some aspects, OCT is used in retinal imaging in ophthalmology and in the imaging of various anatomical structures like the skin, gastrointestinal tract, respiratory system, genitourinary tract, and oral cavity. In certain embodiments, OCT may be used for early cancer detection by distinguishing between tumorous and non-tumorous tissues due to its high resolution and significant penetration depth (up to 2 to 3 mm). OCT can also guide biopsies and aid surgeons with intraoperative cancer imaging by providing immediate feedback. It also proves valuable for monitoring tumor responses to treatments such as radiotherapy, chemotherapy, photodynamic therapy (PDT), and ablative therapies.

Fluorescence imaging (FI) is widely used for visualizing cells, tissues, and biological processes within living organisms. Fluorescence images are generated through microscopy, imaging probes, and spectroscopy. Near-infrared (NIR) fluorescence imaging, in particular, has become integral to tumor-specific fluorescence-guided surgery (TS-FGS). NIR fluorescence delivers high-resolution images and can penetrate tissues and blood several hundred microns deep due to the optimal optical window of 650 to 1350 nm, which minimizes scattering and autofluorescence from biomolecules. With these capabilities, NIR fluorescence imaging offers real-time guidance to surgeons during procedures, assisting in locating structures like tumor margins and sentinel lymph nodes that require resection.

In some embodiments, the imaging is FI and the imaging agent is an NIR dye. In some embodiments, the NIR dyes have an absorption wavelength between the range of about 600 nm to about 1200 nm. In some aspects, the NIR dyes have an absorption wavelength between the range about 600 nm to about 1000 nm, alternatively about 670 nm to about 850 nm, alternatively between about 650 nm to about 900 nm, alternatively between about 650 nm to about 1000 nm, or alternatively about 800 nm. In some aspects, the NIR dyes have an absorption and emission maxima between about 500 nm and about 900 nm, alternatively between about 600 nm and about 800 nm, alternatively between about 650 nm and about 900 nm, alternatively between about 600 nm and about 1000 nm, or alternatively about 800 nm.

In some aspects, the NIR dye is IR800CW. IR800CW is excited by the laser and emission captured by the 780/60 filter. In some aspects, IR800CW has the following structure:

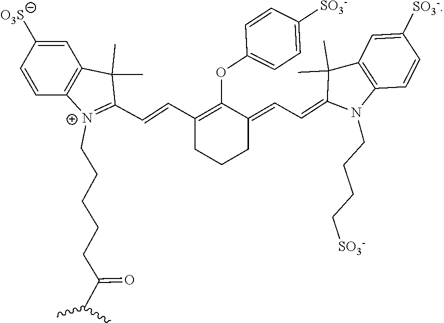

In some aspects, the NIR dye is S0121. In some aspects, S0121 has the following structure:

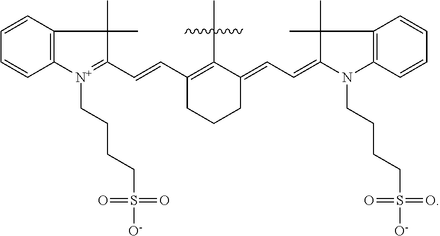 [SMILES: *C1=C(/C=C/C2=[N+](CCCCS(=O)(=O)[O-])c3ccccc3C2(C)C)CCC/C1=C\C=C1\N(CCCCS(=O)(=O)[O-])c2ccccc2C1(C)C]

In some aspects, the NIR dye is S0456. S0456 is a near-infrared (NIR) fluorescent dye with an excitation (Ex) of 788 nM and emission (Em) of 800 nM. In some aspects, S0456 has the following structure:

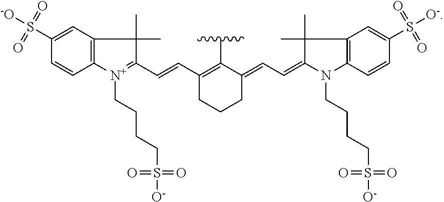

In some aspects, the NIR dye is IRD78. In some aspects, IRD78 has the following structure:

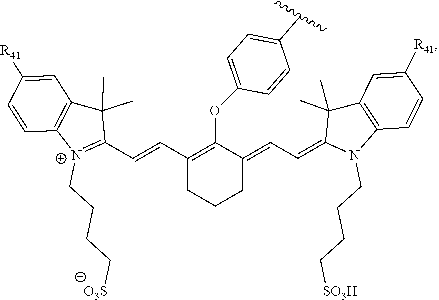

wherein R41 is H or SO₃H.

In some aspects, the NIR dye is ZW800. In some aspects, ZW800 has the following structure:

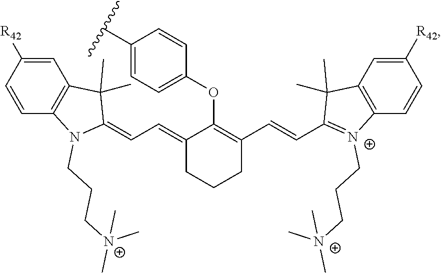

wherein R₄₂ is H or SO₃H.

In some aspects, the NIR dye is Kodak. In some aspects, Kodak has the following structure:

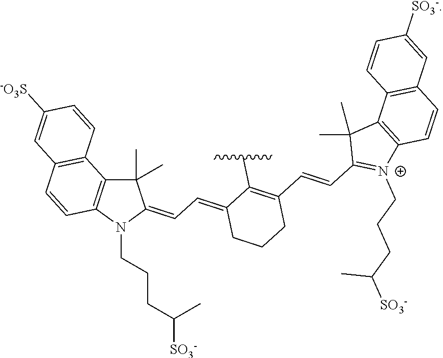

In some aspects, the NIR dye is S2076. In some aspects. S2076 has the following structure

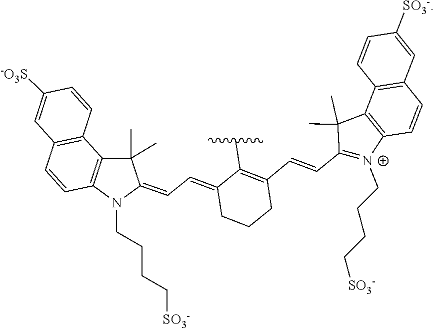

In some aspects, the NIR dye is

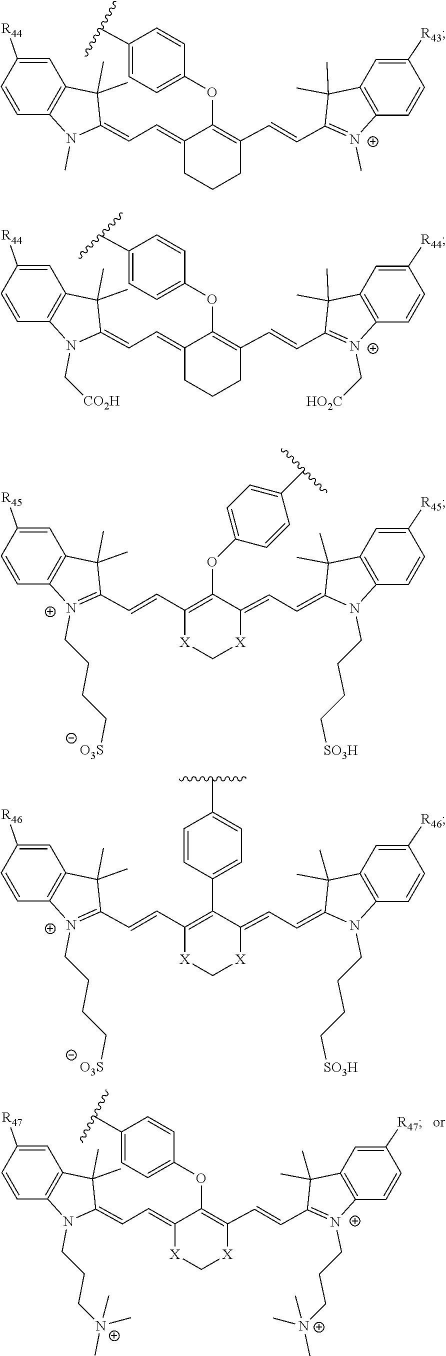

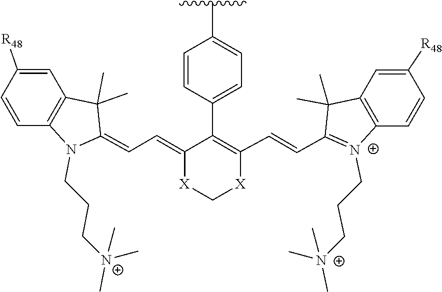

wherein R43, R44, R45, R46, R47, R48=H or SO_{3H}; X═O, S, or N.

### III. Fibroblast Activation Protein (FAP) Targeting Moieties

FAP is a non-classical serine protease belonging to “Dipeptidyl peptidase (DPP)—IV activity and/or structure homologues” (DASH). The FAP gene is highly conserved across various species. It is localized on the long arm of chromosome 2 in humans and mice adjacent to its closest homologue DPP-IV/CD26 (52% amino acid identity) and, similarly to DPP-IV, is organized into 26 exons. It is therefore thought to have arisen by gene duplication. The protein encoded by the human FAP gene is a 760 amino acid single pass type II transmembrane protein composed of a short cytoplasmic N terminal part (6 amino acids), a transmembrane region (amino acids 7-26), and a large extracellular domain. Several isoforms of FAP have been reported in the literature. A soluble form of FAP which lacks 26 amino acids of the intracellular and transmembrane portion can be detected in the plasma in various species and is speculated to be the product of protein shedding. Two alternatively spliced, in-frame mRNA variants have been identified in mouse embryonic tissues. Predicted proteins encoded by these mRNAs contain a transmembrane domain and a catalytic region but lack 33 and 5 amino acids respectively, in the membrane-proximal portion of the protein. Goldstein et al. described a shortened isoform corresponding to the 239 carboxyterminal amino acids of a human FAP protein generated from an alternatively spliced mRNA in melanoma cells. It is not clear, however, whether this isoform is also expressed in vivo. The same group has later reported shortened forms of the human FAP/seprase produced by a proteolytic processing by EDTA-sensitive activators, especially in ovarian carcinoma. These isoforms may exhibit increased collagenolytic activity, possibly due to a reduced steric hindrance for larger substrates. We have recently described the existence of several molecular forms of FAP of varying pI and electrophoretic mobility in human glioblastoma tissues but, like the abovementioned isoforms, their pathophysiological role is currently unknown.

FAP is enzymatically active as a homodimer. It exhibits both a post-proline dipeptidyl peptidase and endopeptidase activity, both of which are dependent on the catalytic triad comprising Ser624 Asp702 His734 in human and mouse FAP. Due to the unique structure of proline, most proteases do not cleave the peptide bonds adjacent to it. In several cases, the presence of proline thus acts as a mechanism that prevents protein degradation or cleavage. Several bioactive peptides and structural proteins have been proposed to be FAP substrates. Of these, neuropeptide Y (NPY), Peptide YY, Substance P (SP), and B-type natriuretic peptide (BNP) are cleaved rapidly, whereas the incretins glucagonlike peptide-1 (GLP-1) and glucose-dependent insulinotropic peptide (GIP), as well as some other biopeptides (GLP-2, Peptide Histidine-Methionine, Growth hormone-releasing hormone) are rather poor substrates. Among structural proteins, collagen I and III have been shown to be cleaved by FAP, but efficient FAP activity seems to require denaturation or predigestion by other proteases. Cleavage of the proteoglycan brevican has also been reported, but only after a prolonged incubation. Substrates that are cleaved in vivo include human fibroblast growth factor 21 (FGF-21), a protein involved in the regulation of energy metabolism and insulin sensitivity, human alpha2 antiplasmin, and probably also collagen I. Further studies are needed to clarify the role of FAP in the proteolysis of other possible substrates. The intracellular antagonist of receptor tyrosine kinases sprouty (SPRY2) is cleaved more efficiently than alpha2 antiplasmin and has been used to study FAP substrate specificity in a study by Huang et al. The physiological relevance, if any, of this cleavage is unclear. Similarly, the various candidate FAP substrates identified by proteomic approaches (ADAM15, interleukin 6 (IL-6), fibrillin-2, matrillin-3, serine protease 23, testican-1, and transforming growth factor beta-induced protein) remain to be validated, and the physiological importance of their cleavage by FAP is yet to be established.

FAP expression has been documented in some of the primitive mesenchymal cells at various stages of mouse embryonic development, but its absence did not lead to developmental defects. Although most normal adult tissues show little or no detectable FAP expression, a soluble form of FAP is present in the blood plasma of various species. In humans, plasma concentrations of FAP measured by ELISA in healthy individuals are around 100 ng/ml or 0.6 nmol/l (median concentration reported in the range of 15-500 ng/ml in various studies depending on the material and methodology used). In mice, plasma FAP enzymatic activity is higher than in humans. The source of plasma FAP is at present unknown. In healthy adult humans, FAP is co-expressed with DPP-IV in the alpha cells of Langerhans islets. It is also present in multipotent bone marrow stromal cells (BM-MSC) in both mice and humans. Independent of its enzymatic activity, FAP promotes the motility of human BM-MSC possibly by regulating the RhoA activity. FAP protein is also weakly expressed in the cervix and in the uterine stroma, where it reaches the highest levels during the proliferative phase. It has also been detected in the human placenta and in some cases in dermal fibroblasts surrounding hair follicles.

Increased FAP expression is associated with several non-malignant conditions, especially those that involve tissue remodeling. Skin wound healing induces FAP expression in fibroblasts, and increased FAP was also reported in keloids and in scleroderma. Similarly, healing after myocardial infarction is accompanied by the presence of FAP+ activated fibroblasts and FAP contributes to their migratory potential. FAP expression has also been detected in the submucosa and muscle layer in intestinal strictured regions in Crohn's disease, in advanced aortic atherosclerotic plaques, and in thincap human coronary fibroatheromata, where it was proposed to contribute to type I collagen breakdown in the fibrous caps.

FAP is undetectable in a healthy liver, but markedly elevated in liver cirrhosis. FAP is expressed predominantly in the hepatic stellate cells (HSC) at the tissue remodeling interface around regenerative nodules, where it co-localizes with collagen I and fibronectin. A weaker expression has also been detected in the cells of the fibrous portal septa. Further work has demonstrated that the intensity of FAP immunoreactivity correlates with the severity of liver fibrosis in hepatitis C infected patients and its serum concentrations are elevated in patients with alcoholic liver disease. Functionally, FAP may, independently of its enzymatic activity, increase the adhesion, migration, and apoptosis in the HSC.

FAP is not detectable in normal human lung or centriacinar emphysema by immunohistochemistry. FAP is expressed in idiopathic pulmonary fibrosis, particularly in areas of ongoing tissue injury (fibroblast foci in close association with hyperplastic epithelium), but it is absent in the neighboring normal tissue. Interestingly, FAP seems to have a protective role in the context of idiopathic pulmonary fibrosis. Using FAP knockout mice, Fan et al. have demonstrated that the absence of FAP led to a decrease in collagen I fragment clearance from the lungs, and thereby also an increase in the fibrotic response and decreased animal survival.

In osteoarthritis and rheumatoid arthritis, FAP expression has been demonstrated in fibroblast-like synoviocytes. In rheumatoid arthritis, higher FAP levels have been observed in connection with increased levels of other proteins involved in extracellular matrix turnover (matrix metalloproteinases (MMP) 1 and 13, and CD44 splice variants v3 and v7/8). In osteoarthritis of the hip, FAP expression has been further detected in the chondrocytes in the superficial zone of the cartilage. It was induced by interleukin 1 (IL-1) and oncostatin M, cytokines which promote cartilage destruction. In a model of murine arthritis, radiolabeled anti-FAP antibodies accumulated in inflamed joints and signal intensity correlated with the severity of the inflammation and with response to treatment. In addition, FAP knockout mice exhibited a decrease in cartilage destruction. FAP+ cells thus probably contribute to joint destruction and FAP seems to be involved in these processes.

FAP was originally identified as an antigen recognized by the F19 murine monoclonal antibody raised against lung fibroblasts. Seminal works by Rettig et al. suggested that this antigen was expressed in a large proportion of astrocytoma, sarcoma, and melanoma cell lines, as well as in cultured normal fibroblasts in vitro, whereas epithelial cells, including malignant epithelial cells and hematopoietic malignancies, were F19 negative. On the tissue level, it has been shown that the antigen is a characteristic trait of the stroma in various malignancies, while its expression under physiological conditions is very limited. Reflecting its predominant localization in activated fibroblasts, the abovementioned investigators coined the designation “fibroblast activation protein”.

Further work expanded the list of malignancies that characteristically overexpress FAP and revealed that in addition to cancer-associated fibroblasts, FAP may be present in other cellular components of the tumor microenvironment. FAP has been detected in endothelial cells, in a subpopulation of CD45+ stromal cells (presumably macrophages), and osteoclasts in multiple myeloma. Association between FAP expression and clinicopathological variables, including patient survival, seems to be tumor type-dependent, but larger studies have not yet been undertaken. A recently published meta-analysis involving 15 studies which assessed FAP expression in 11 solid cancers by immunohistochemistry concluded that FAP positivity is found in 50-100% of patients and a higher FAP expression is associated with 1) a higher local tumor invasion, 2) increased risk of lymph node metastases, 3) decreased survival, in particular in cases where FAP is expressed in the malignant cells. The association with a worse survival has been most clearly demonstrated in colorectal and pancreatic carcinoma, but was also reported for hepatocellular, ovarian, non-small cell lung carcinoma, and osteosarcoma. Nonetheless, a recent retrospective study in pancreatic cancer by Park et al. suggests that a high number of FAP+ fibroblasts is associated with increased overall survival. In breast cancer, improved prognosis in patients with FAP positive stroma has been reported, but other studies could not confirm this finding. The cause of these somewhat discrepant results is currently unclear but may involve differences in the methodology of FAP quantification as well as differences in the FAP/seprase epitopes recognized by various antibodies, in particular in paraffin sections. It is thought that FAP participates in several hallmarks of malignancy, including transformed cell invasiveness and proliferation, extracellular matrix (ECM) remodeling, tumor vascularization, and escape from immunosurveillance. The gelatinolytic activity of FAP contributes to ECM degradation. Several reports have shown that FAP is localized mainly in the invadopodia of migrating cells, where it forms proteolytic complexes (e.g. with DPP-IV or urokinase receptor (uPAR)). Integrins such as alpha3 beta1 interact with FAP and are probably responsible for this specific localization of FAP. In line with these data, it has been shown that FAP contributes to tumor cell motility and invasiveness.

One approach to targeting FAP, is through the use of FAP targeting molecule conjugated imaging or therapeutic agents. In some embodiments, the FAP targeting molecules have the formula:

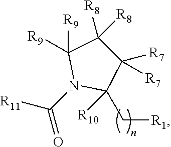

wherein R¹ is CHO, C═CH, CH(CN)₂, CN, NHCN, B(OH)R₂, COR₂, Zn(OH), PO(OH)R₂, SO₂R₂, Si(OH)₂R₂, a halogen, OH, H or COCONHR₃;
wherein R₂ is OH, SH, NH₂, O-alkyl, S-alkyl, NH-alkyl, a halogen, or H; and
R₃ is H, a halogen, OH, methyl, ethyls, isopropyl, tBu, cyclopropyl, alkyl, alkyl-OH, CN, CHPh₂, adamantane or

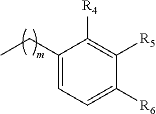

wherein R₄, R₅, or R₆ are independently selected from the group consisting of H, OCH₃, SCH₃, COR₂, SO₂R₂, PO(OH)R₂, Si(OH)₂R₂, halogen, OH, CN, B(OH)R₂, and combinations thereof;
R₇, R₈, R₉, or R₁₀ are independently selected from the group consisting of H, a halogen, OH, OCH₃, SCH₃, CN, COR₂, SO₂R₂, PO(OH)R₂, Si(OH)₂R₂, B(OH)R₂, a C₁-C₁₀ alkyl, and combinations thereof;
n and m are independently selected from 0, 1, 2, 3, 4, or 5;
R₁₁ is selected from the group consisting of

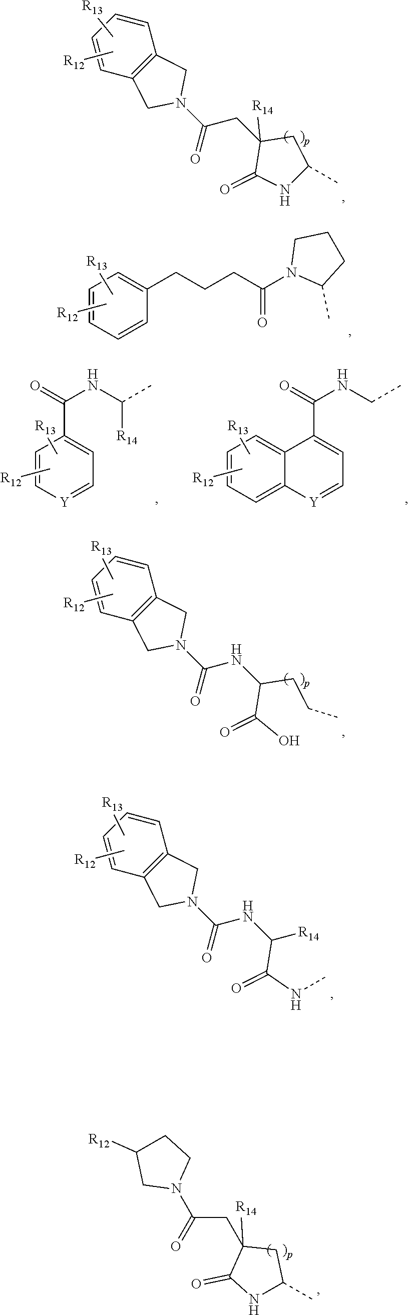

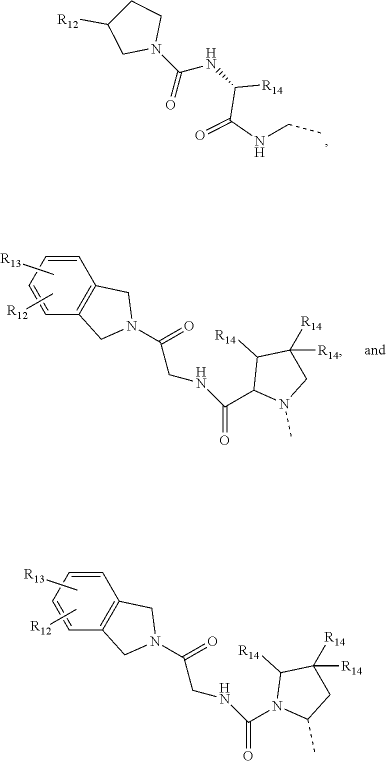

wherein R₁₂ is (CH2)q-X, X═COOH, SH, OH, NH₂, halogen, or maleimide;
R₁₃ is H, halogen, Me, OH, SH, OCH₃, OBn, or CH₂OH;
R₁₄ is H, F, CH₃, OCH₃, Isopropyl, or Tert-butyl;
Y is CH or N; and
p and q are independently selected from 0, 1, 2, 3, 4, or 5.

### IV. FAPI—Image-Guided Surgery Agents

The disclosure describes several novel FAPI-image-guided surgery agents. These agents leverage targeting activities of the disclosed FAP inhibitors and the high sensitivity disclosed dyes. In some embodiments, the FAPI-image-guided surgery agents have the following formula B—X—Z, wherein B comprises a FAP-targeted molecule (or FAP inhibitor), X comprises a spacer, and Z comprises an imaging agents.

In some aspects, B is a monovalent group derived from a FAP-targeted molecule. In some aspects, the FAP-targeted molecule is selected from the group consisting of a small molecule, a ligand, an inhibitor, an agonist, or a derivative thereof. In some aspects, the FAP-targeted compound is a ligand. In other aspects, the FAP-targeted compound is a small molecule that binds FAP. In some aspects, the FAP-targeted compound is a small molecule with an extended hydrophobic moiety with alpha keto amide unit. In some aspects, the FAP-targeted compound is a small molecule with an substituted alpha keto amide. In some aspects, the FAP-targeted compound is a small molecule with an substituted alpha keto amide with aromatic moiety with methoxy units.

In some aspects B has the formula:

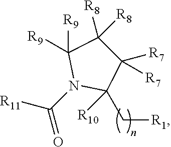

wherein R₁ is CHO, C═CH, CH(CN)₂, CN, NHCN, B(OH)R₂, COR₂, Zn(OH), PO(OH)R₂, SO₂R₂, Si(OH)₂R₂, a halogen, OH, H or COCONHR₃;
wherein R₂ is OH, SH, NH₂, O-alkyl, S-alkyl, NH-alkyl, a halogen, or H; and
R₃ is H, a halogen, OH, methyl, ethyls, isopropyl, tBu, cyclopropyl, alkyl, alkyl-OH, CN, CHPh₂, adamantane or

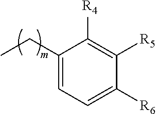

wherein R₄, R₅, or R₆ are independently selected from the group consisting of H, OCH₃, SCH₃, COR₂, SO₂R₂, PO(OH)R₂, Si(OH)₂R₂, halogen, OH, CN, B(OH)R₂, and combinations thereof;
R₇, R₈, R₉, or R₁₀ are independently selected from the group consisting of H, a halogen, OH, OCH₃, SCH₃, CN, COR₂, SO₂R₂, PO(OH)R₂, Si(OH)₂R₂, B(OH)R₂, a C₁-C₁₀ alkyl, and combinations thereof;
n and m are independently selected from 0, 1, 2, 3, 4, or 5;
R₁₁ is selected from the group consisting of

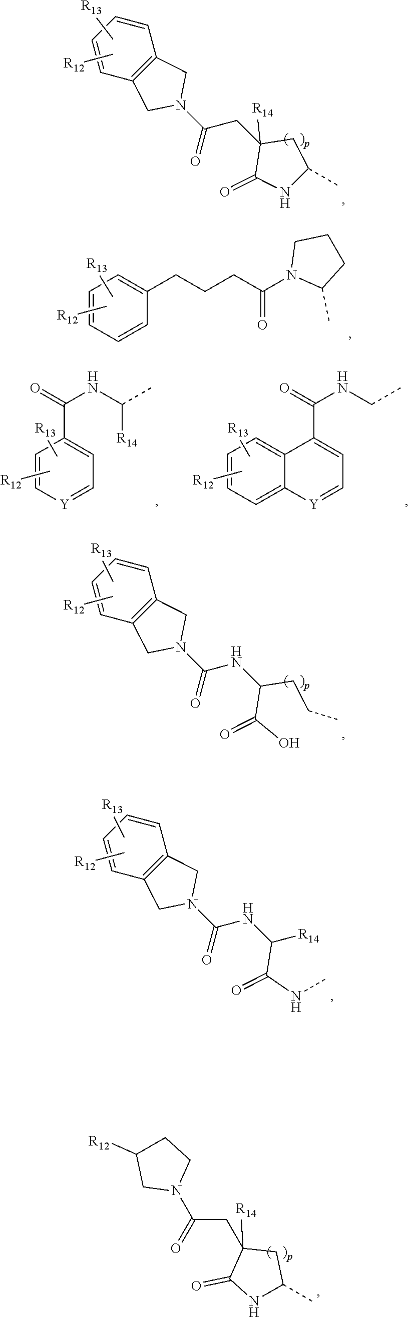

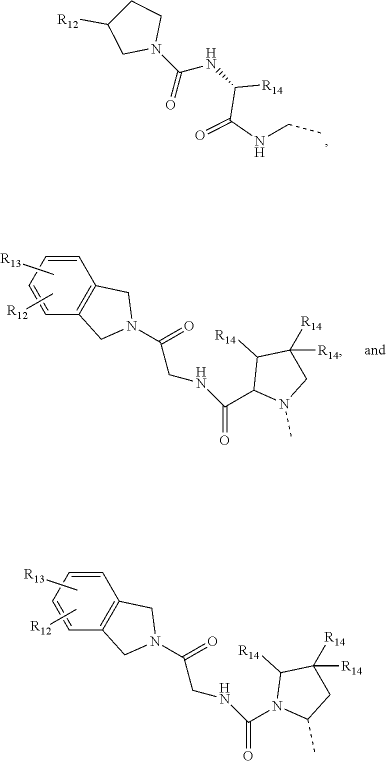

wherein R₁₂ is (CH₂)q-X, X=COOH, SH, OH, NH₂, halogen, or maleimide;
R₁₃ is H, halogen, Me, OH, SH, OCH₃, OBn, or CH₂OH;
R₁₄ is H, F, CH₃, OCH₃, Isopropyl, or Tert-butyl;
Y is CH or N; and
p and q are independently selected from 0, 1, 2, 3, 4, or 5.

In some aspects, compounds which include a linker (i.e., X) having a predetermined length, and/or a predetermined diameter, and/or preselected functional groups along its length may provide enhanced imaging properties when compared to other imaging agents.

In some aspects, X is a hydrophobic spacer. In some aspects, X is variably charged, has a negative charge, or has a positive charge.

In some aspects, X is selected from the group consisting of six aminohectanoic acid (SAHA), eight aminooctonoic acid (EAOA), polyethylene glycol (PEG), polyethylene amine (PEA) unit, N-amino-dPEG₂ acid. In some aspects, X is a peptide comprising at least one aryl group or at least one aryl alkyl group, each of which is optionally substituted. In another aspect, the peptide comprises at least two aryl or aryl alkyl group, wherein the first aryl or aryl alkyl group is about 6 atoms to about 10 atoms, alternatively about 6 to about 14 atoms, and the second aryl or aryl alkyl group is about 10 atoms to about 14 atoms, alternatively about 10 to about 15 atoms.

In an aspect, X comprises a peptide comprising at least one amino acid selected from the group consisting of a quaternary amine containing amino acid, an acidic amino acid, a basic amino acid, a neutral polar amino acid, a neutral nonpolar amino acid, an aromatic amino acid, and amino acid derivatives. In another aspect, the acidic amino acid is selected from the group consisting of aspartic acid and glutamic acid. In another aspect, the basic amino acid is selected from the group consisting of arginine, lysine, histidine, and ornithine. In another aspect, the neutral polar amino acid is selected from the group consisting of glycine, serine, threonine, cysteine, tyrosine, asparagine, and glutamine. In another aspect, the neutral nonpolar amino acid is selected from the group consisting of alanine, leucine, isoleucine, valine, proline, phenylalanine, tryptophan, and methionine.

In some aspects, the amino acid derivative is derived from tyrosine. In another aspect, the amino acid derivative is tyramine. In another aspect, the amino acid derivative is selected from the group consisting of:

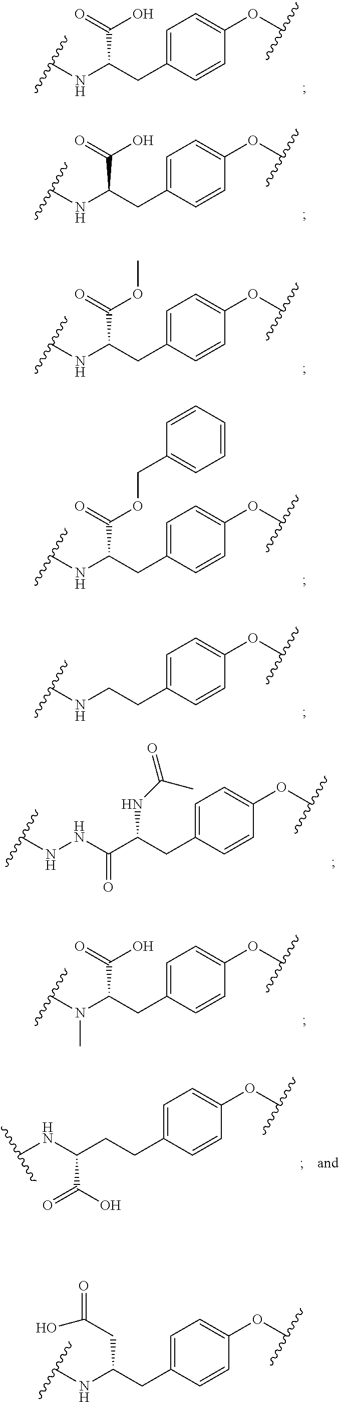

In an aspect, X comprises an amino acid spacer with sulfur-containing side chain group. In another aspect, the sulfur-containing side group is selected from the group consisting of cysteine, methionine and a molecule containing a thiophenol moiety. In an aspect, X comprises an amino acid spacer with a chalcogen-containing side chain group. In another aspect, X comprises a single amino acid selected from the group consisting of tyrosine, cysteine, lysine, tyramine, a tyrosine derivative, a cysteine derivative, and a lysine derivative. In a further aspect, the tyrosine derivative is selected from the group consisting of:

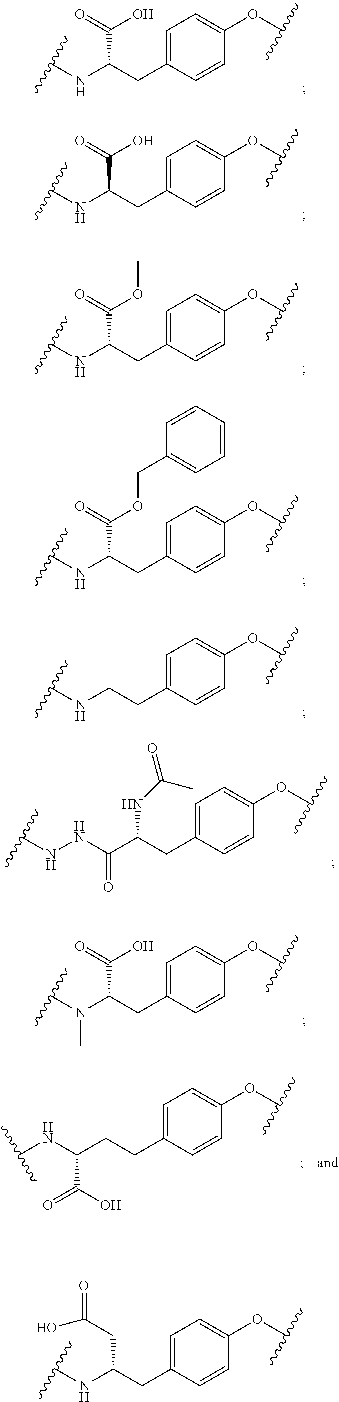

In an aspect, X comprises an amino acid isotope. In another aspect, the amino acid is tyrosine, wherein the tyrosine comprises a hydrogen isotope or carbon isotope on the aromatic ring.

In another aspect, the linker X includes one or more hydrophilic linkers capable of interacting with one or more residues of FAP.

In some aspects, X has a length of about 1 atoms to about 20 atoms, alternatively about 2 atoms, alternatively about 3 atoms, alternatively about 4 atoms, alternatively about 5 atoms, alternatively about 6 atoms, alternatively about 7 atoms, alternatively about 8 atoms, alternatively about 9 atoms, alternatively about 10 atoms, alternatively about 11 atoms, alternatively about 12 atoms, alternatively about 13 atoms, alternatively about 14 atoms, alternatively about 15 atoms, alternatively about 16 atoms, alternatively about 17 atoms, alternatively about 18 atoms, alternatively about 19 atoms, alternatively about 20 atoms.

In one illustrative aspect, the linker X may be a releasable or non-releasable linker. In one aspect, the linker X is at least about 7 atoms in length. In one variation, the linker X is at least about 10 atoms in length. In one variation, the linker X is at least about 14 atoms in length. In another variation, the linker X is between about 7 and about 22, between about 7 and about 20, or between about 7 and about 18 atoms in length. In another variation, the linker X is between about 14 and about 22, between about 15 and about 12, or between about 14 and about 20 atoms in length.

In an alternative aspect, the linker X is at least about 10 angstroms (Å) in length.

In one variation, the linker X is at least about 15 Å in length. In another variation, the linker L is at least about 20 Å in length. In another variation, the linker X is in the range from about 10 Å to about 30 Å in length.

In an alternative aspect, at least a portion of the length of the linker X is about 5 Å in diameter or less at the end connected to the binding ligand B. In one variation, at least a portion of the length of the linker X is about 4 Å or less, or about 3 Å or less in diameter at the end connected to the binding ligand B. It is appreciated that the illustrative aspects that include a diameter requirement of about 5 Å or less, about 4 Å or less, or about 3 Å or less may include that requirement for a predetermined length of the linker, thereby defining a cylindrical-like portion of the linker. Illustratively, in another variation, the linker includes a cylindrical portion at the end connected to the binding ligand that is at least about 7 Å in length and about 5 Å or less, about 4 Å or less, or about 3 Å or less in diameter.

It is to be understood that the foregoing aspects and aspects may be included in the linker X either alone or in combination with each other. For example, linkers X that are at least about 7 atoms in length and about 5 Å, about 4 Å or less, or about 3 Å or less in diameter or less are contemplated and described herein, and also include one or more hydrophilic linkers capable of interacting with one or more residues of FAP, including valine, leucine, phenylalanine, tyrosine, methionine, lysine, cystine, and like residues are contemplated and described herein.

In another aspect, one end of the linker is not branched and comprises a chain of carbon, oxygen, nitrogen, and sulfur atoms. In one aspect, the linear chain of carbon, oxygen, nitrogen, and sulfur atoms is at least 5 atoms in length. In one variation, the linear chain is at least 7 atoms, or at least 10 atoms in length. In another aspect, the chain of carbon, oxygen, nitrogen, and sulfur atoms are not substituted. In one variation, a portion of the chain of carbon, oxygen, nitrogen, and sulfur atoms is cyclized with a divalent fragment.

In some aspects, Z comprises a monovalent group derived from a NIR dye. In some aspects, the NIR dye is selected from the group consisting of:

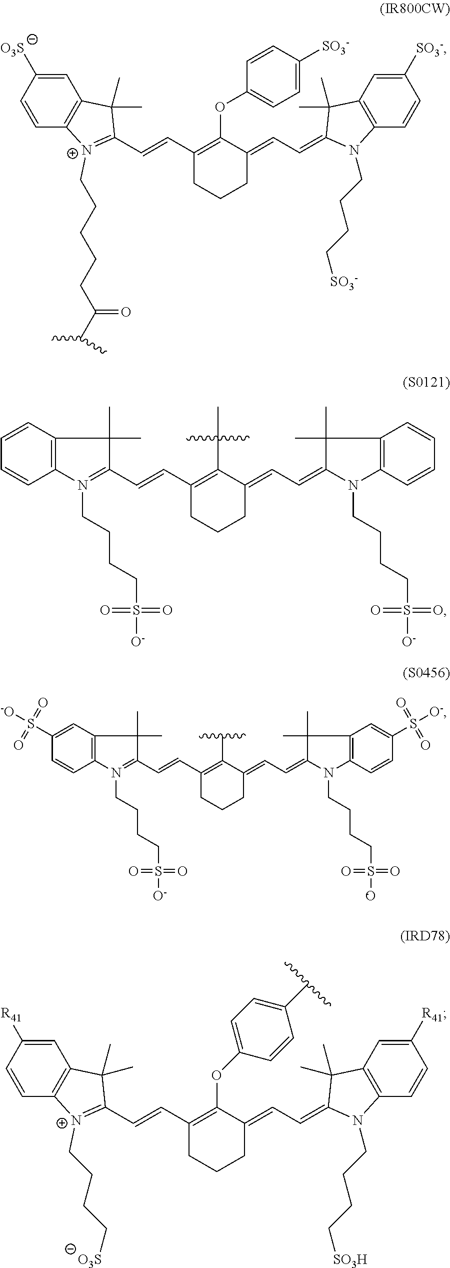

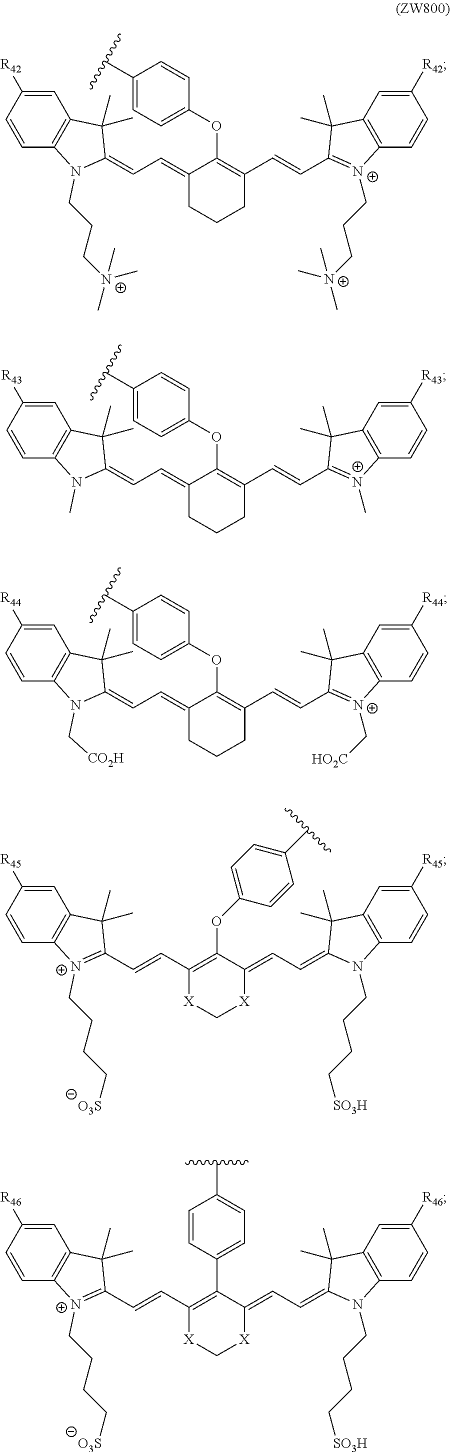

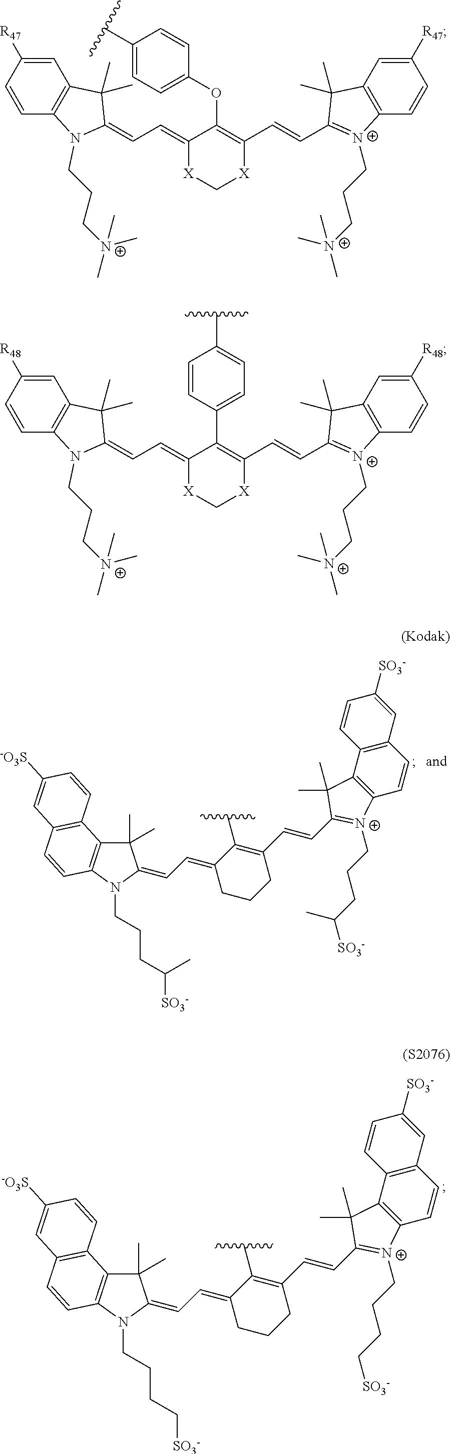

wherein R₄₁, R₄₂, R₄₃, R₄₄, R₄₅, R₄₆, R₄₇, R₄₈ is H or SO₃H and X is O, S, or N.

In some aspects, Z is variably charged, has a negative charge, or has a positive charge.

In some aspects, B comprises a small molecule or a ligand, X comprises eight aminooctonoic acid (EAOA) or polyethylene glycol (PEG), and Z comprises IRD78.

In some aspects, B comprises a small molecule with an substituted alpha keto amide or a small molecule with an substituted alpha keto amide with aromatic moiety with methoxy units, X comprises polyethylene glycol (PEG) or a peptide comprising at least one aryl group or at least one aryl alkyl group, each of which is optionally substituted, and Z comprises IRD78 or S0456.

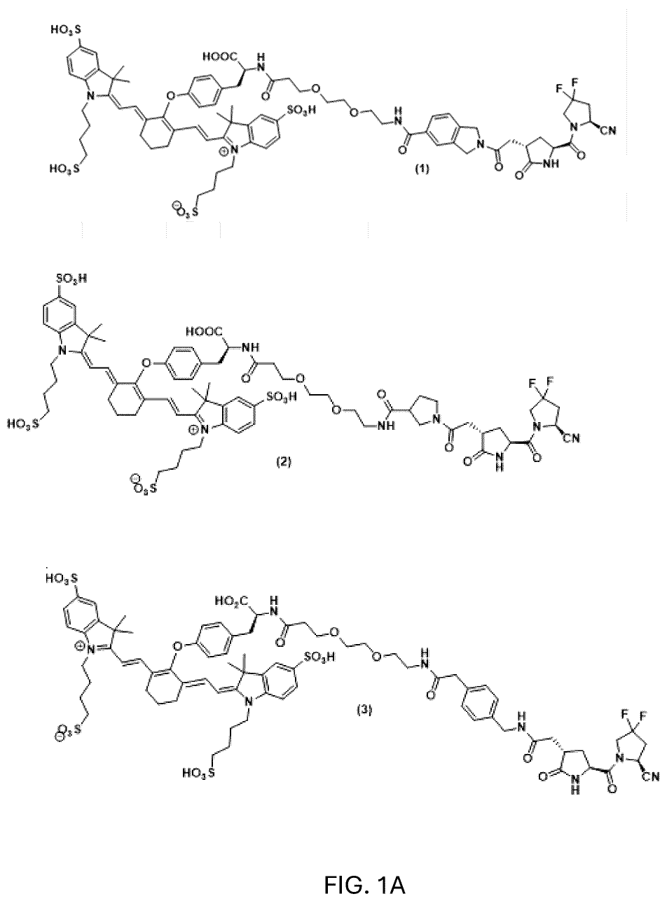

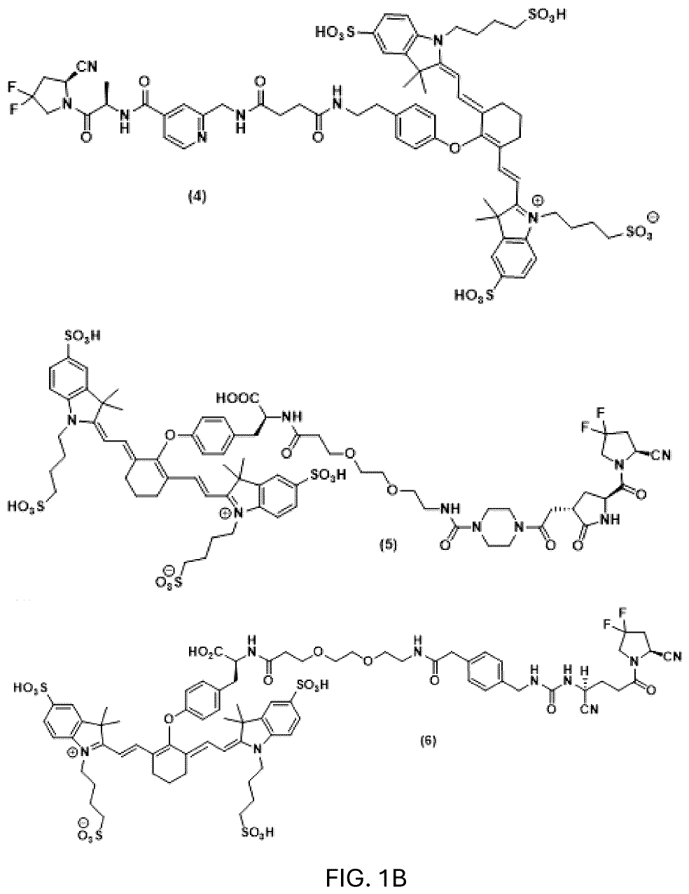

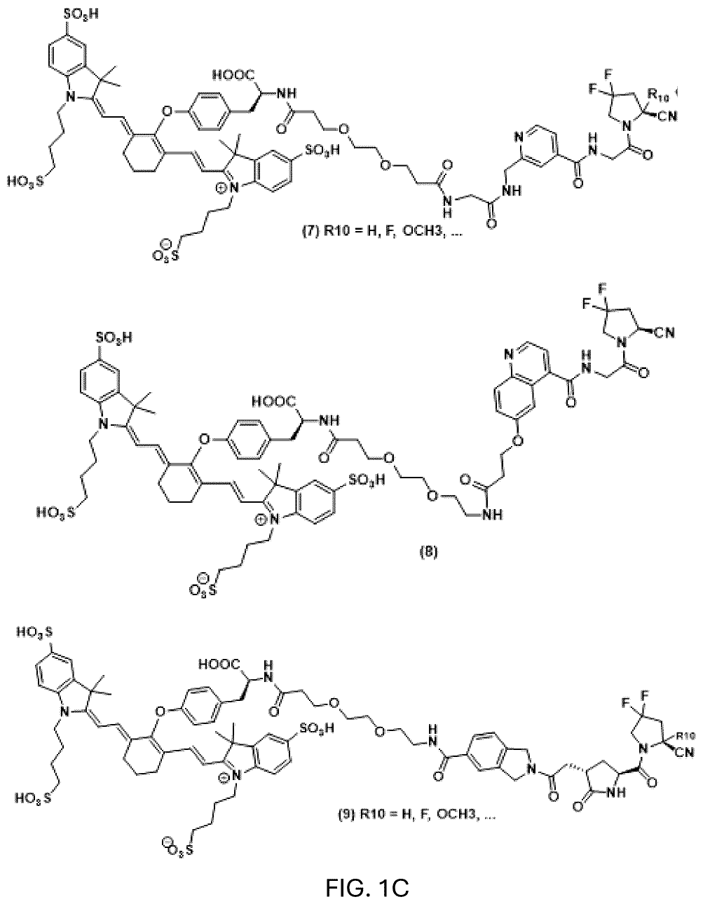

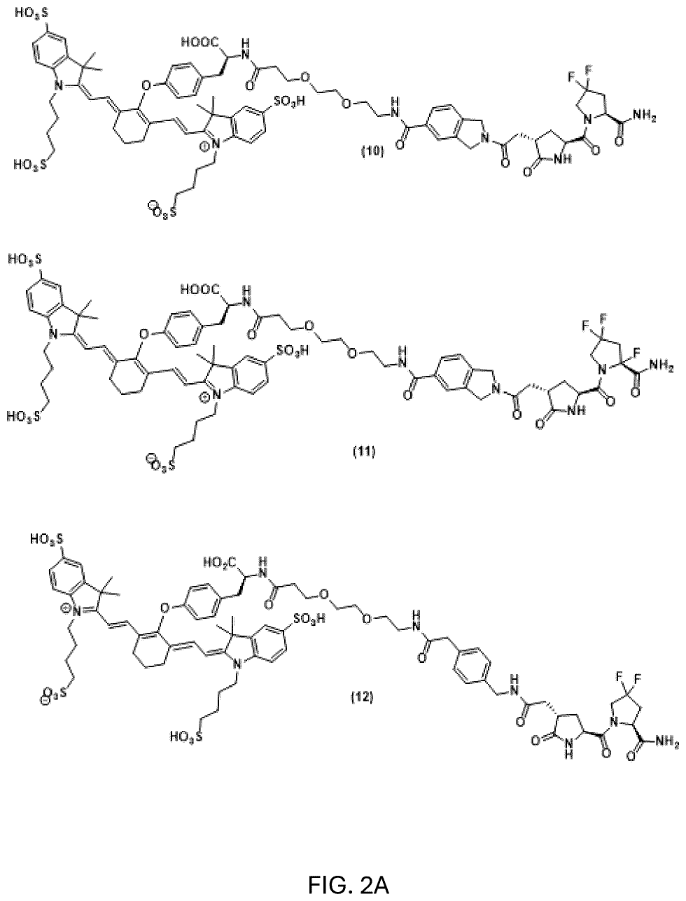

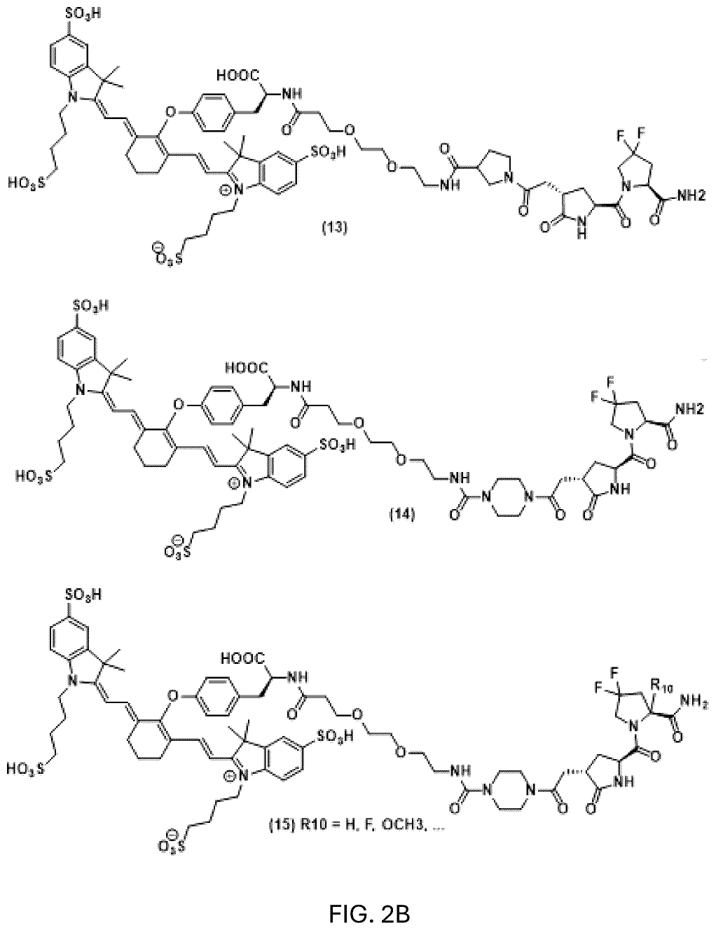

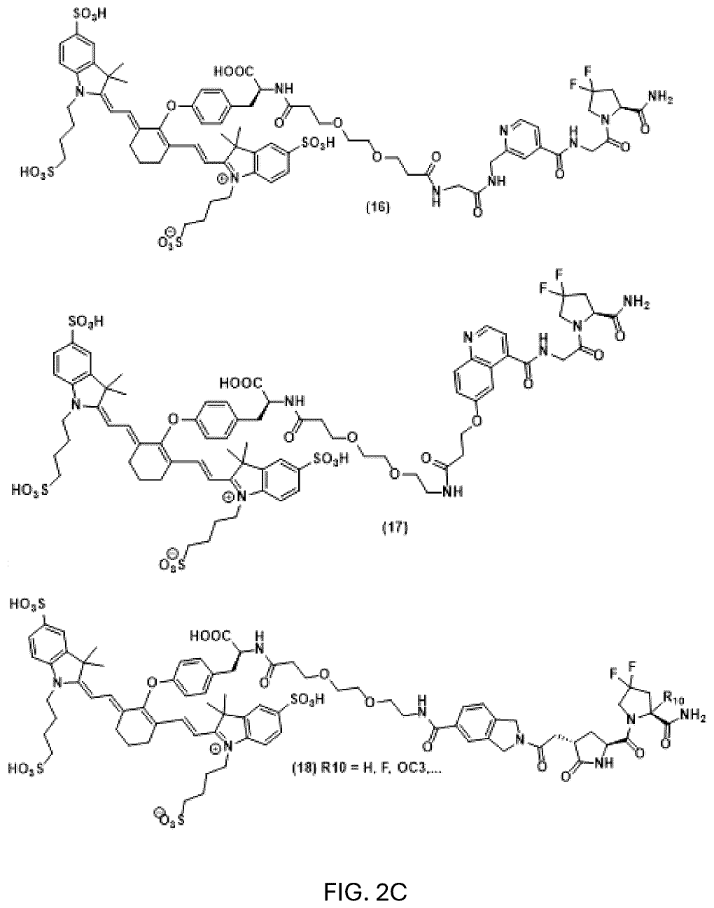

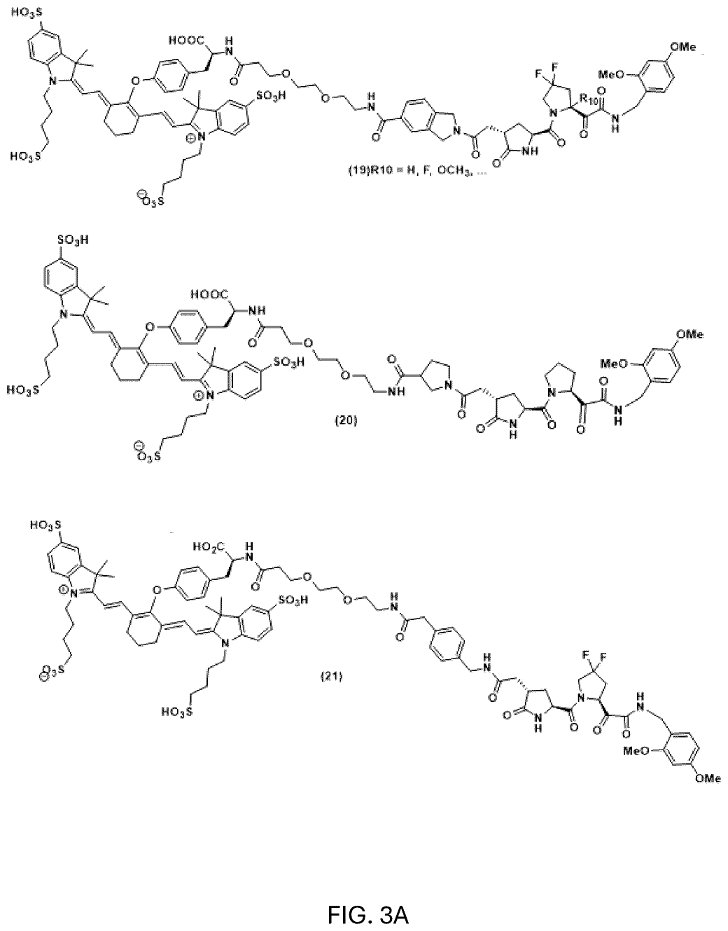

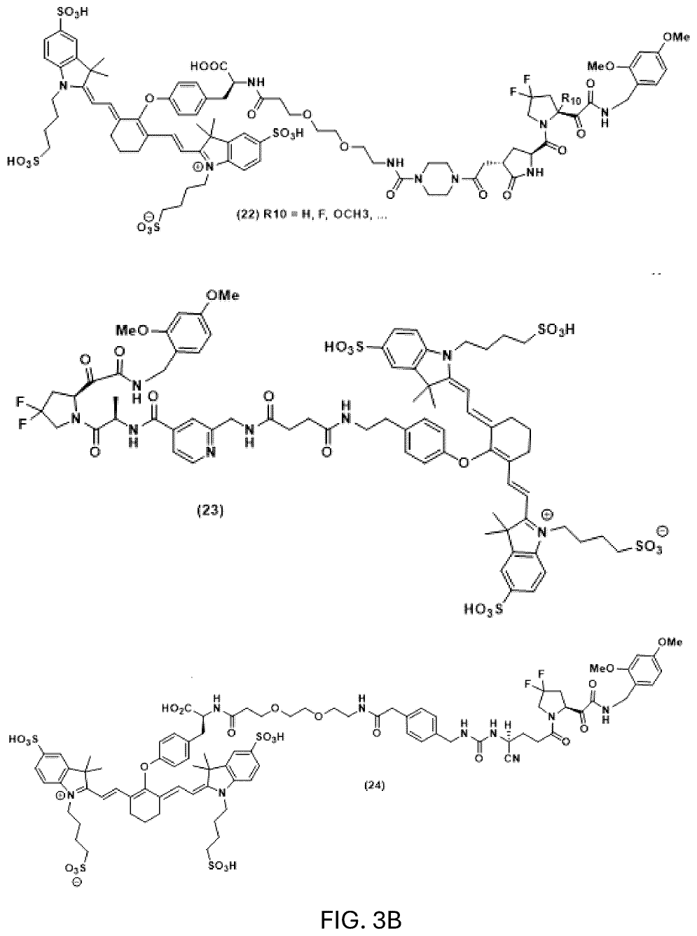

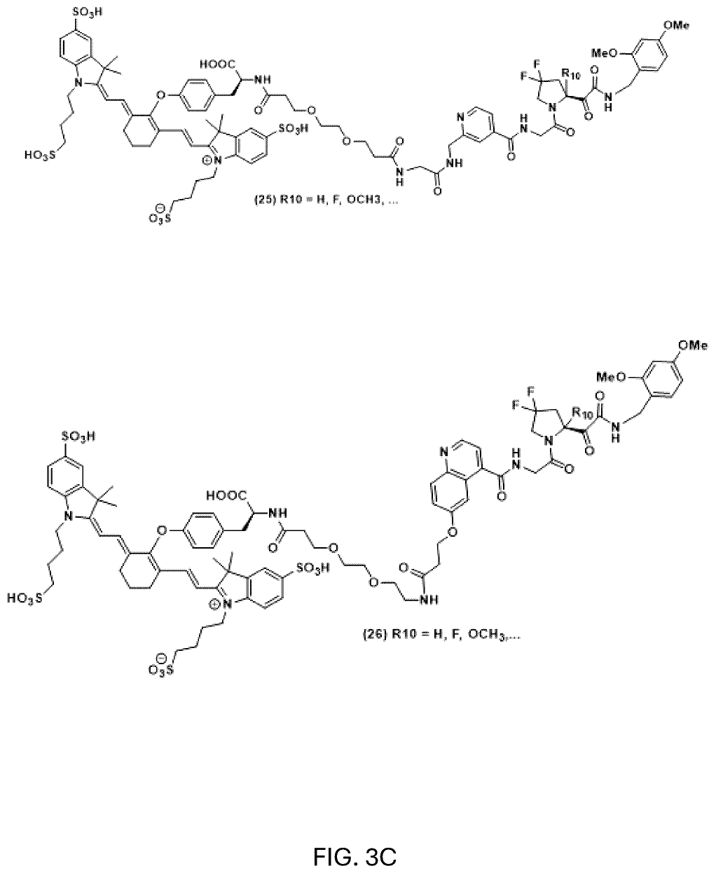

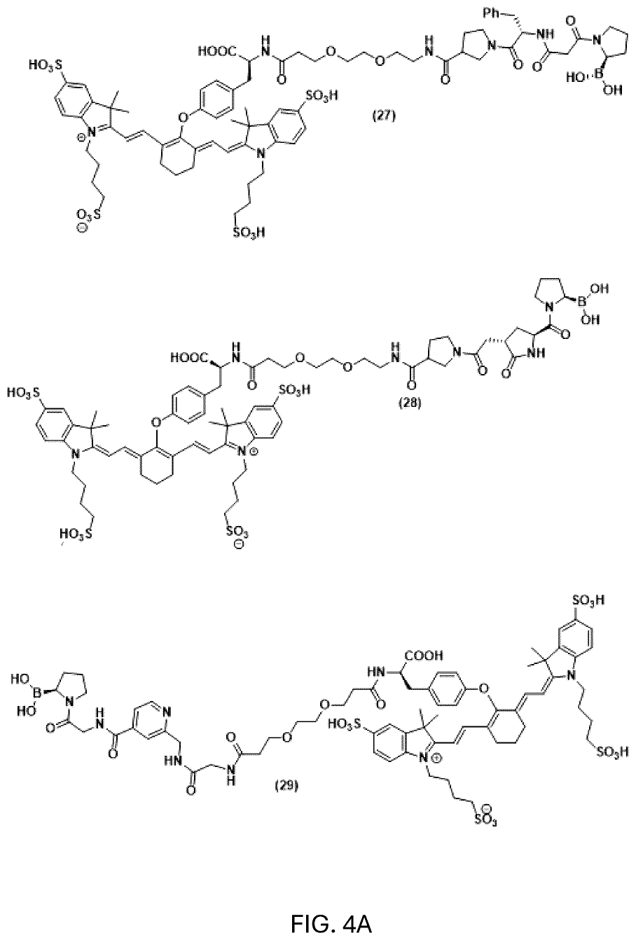

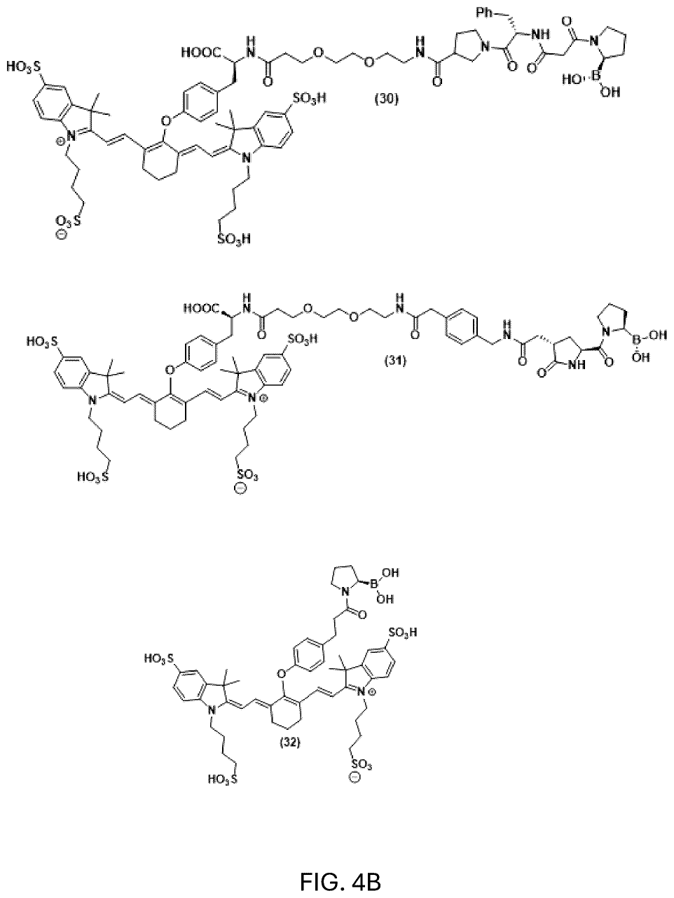

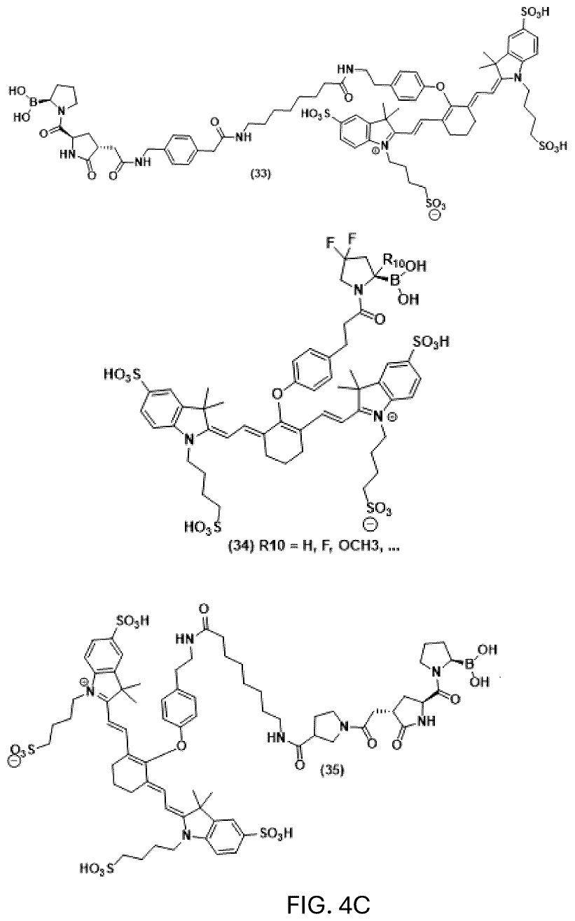

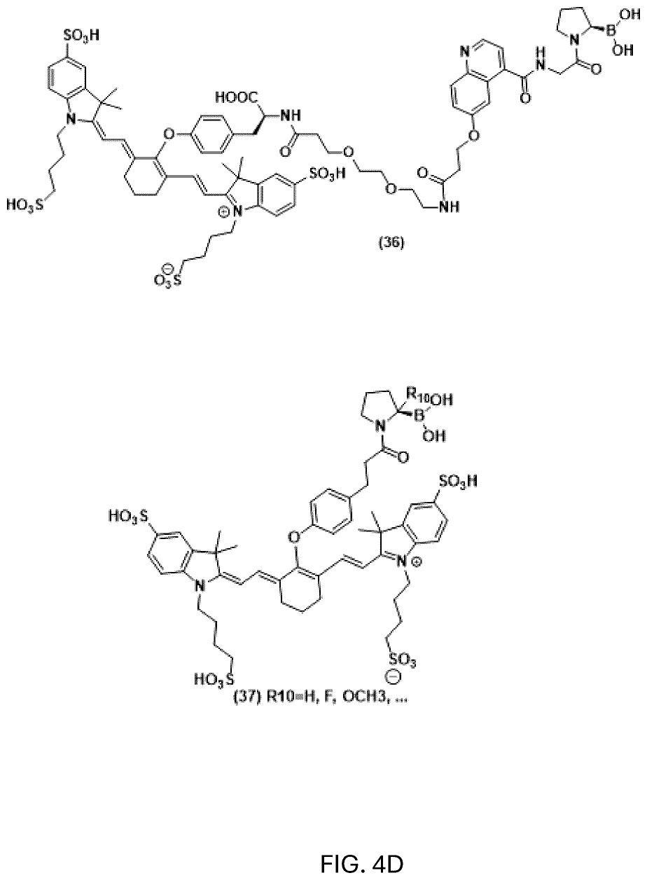

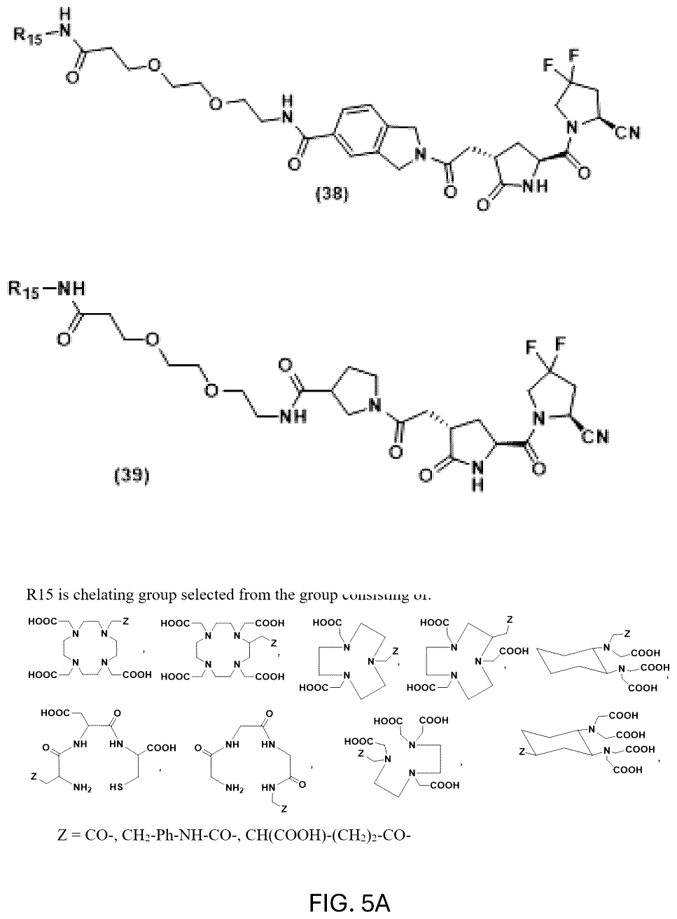

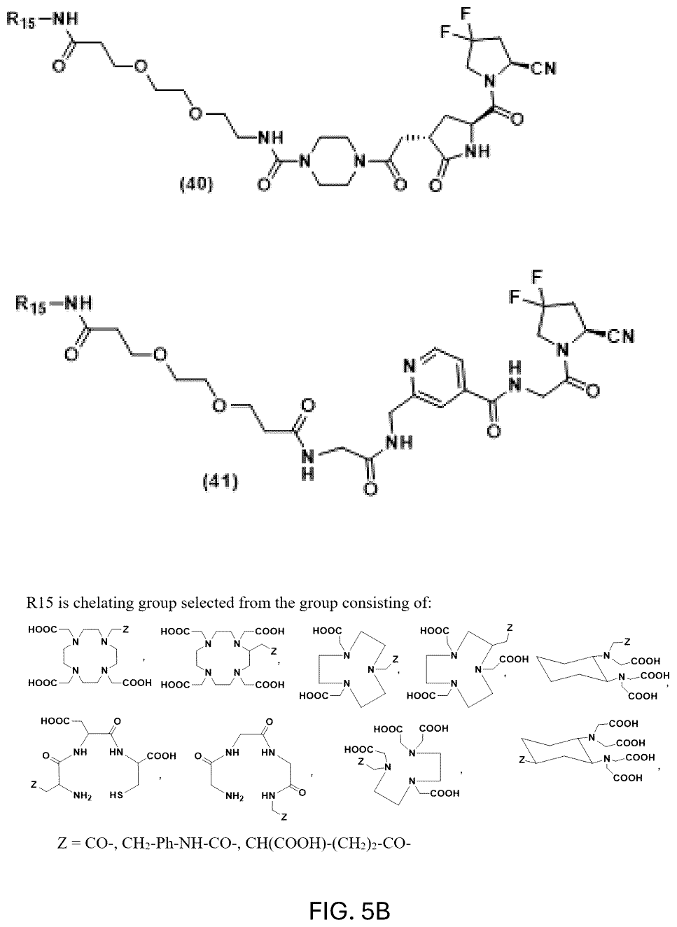

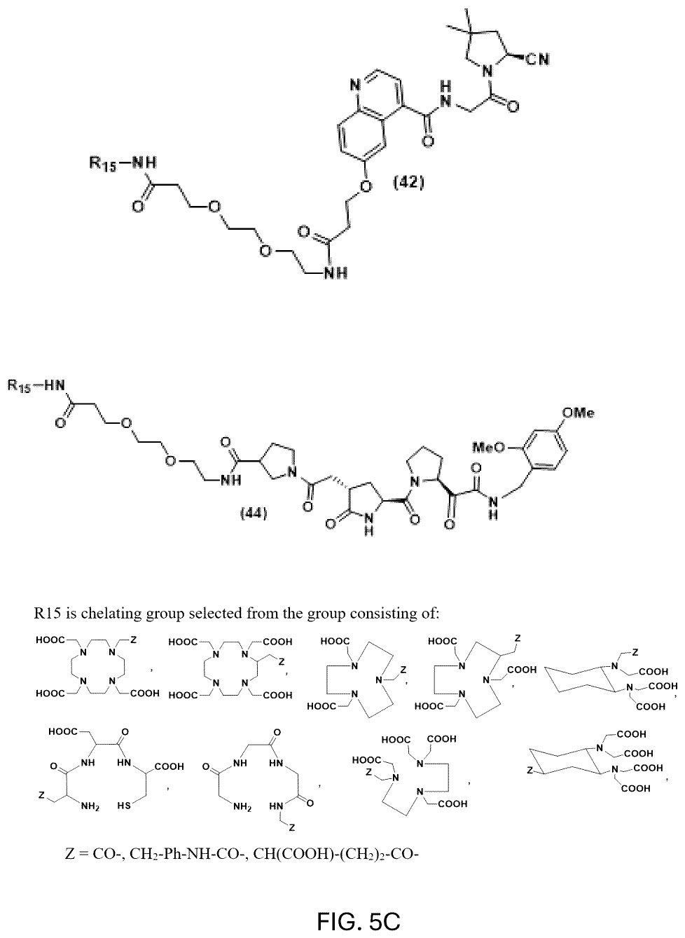

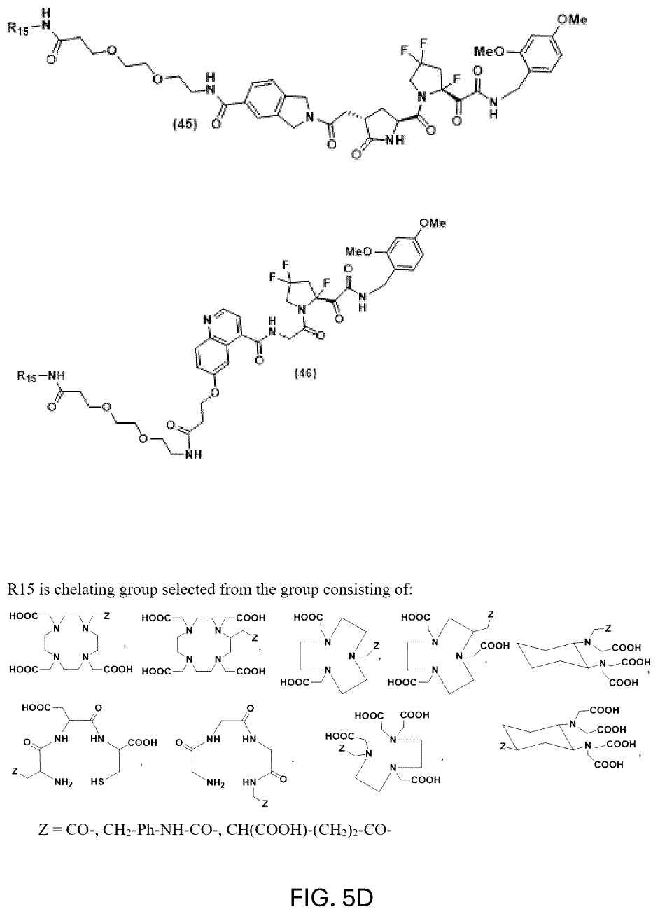

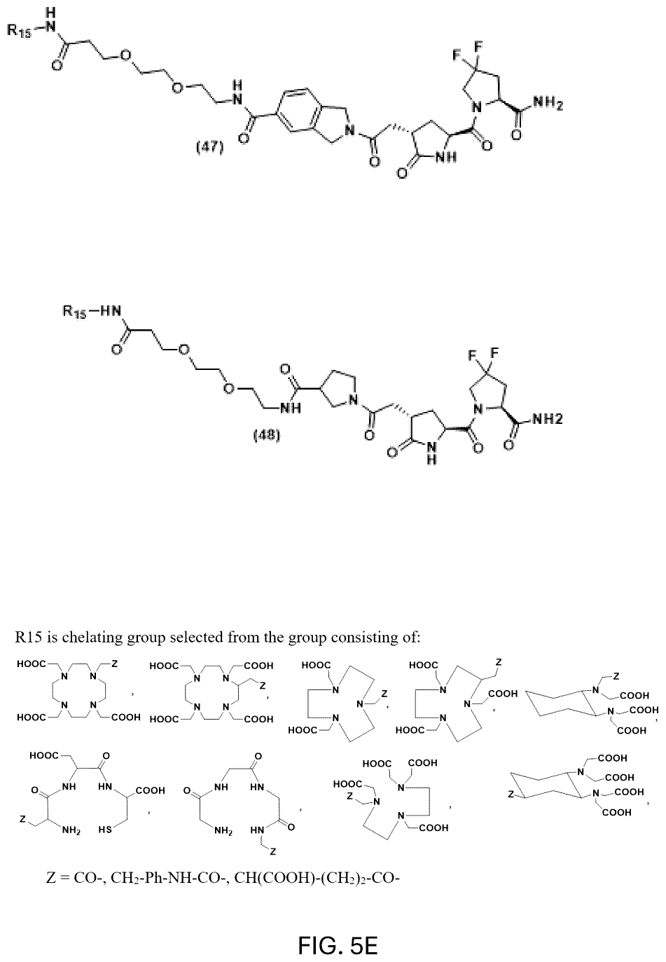

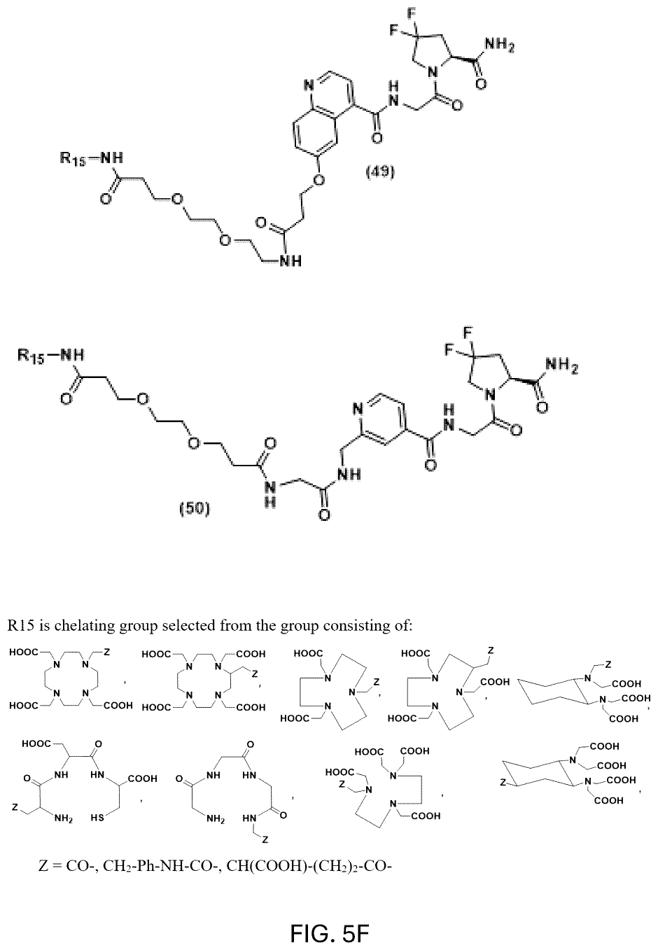

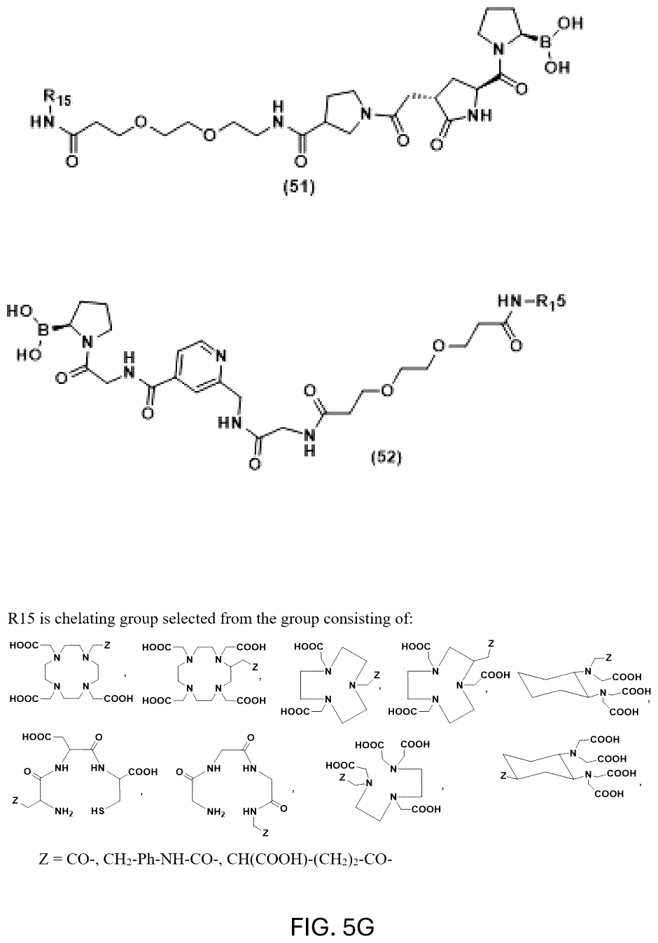

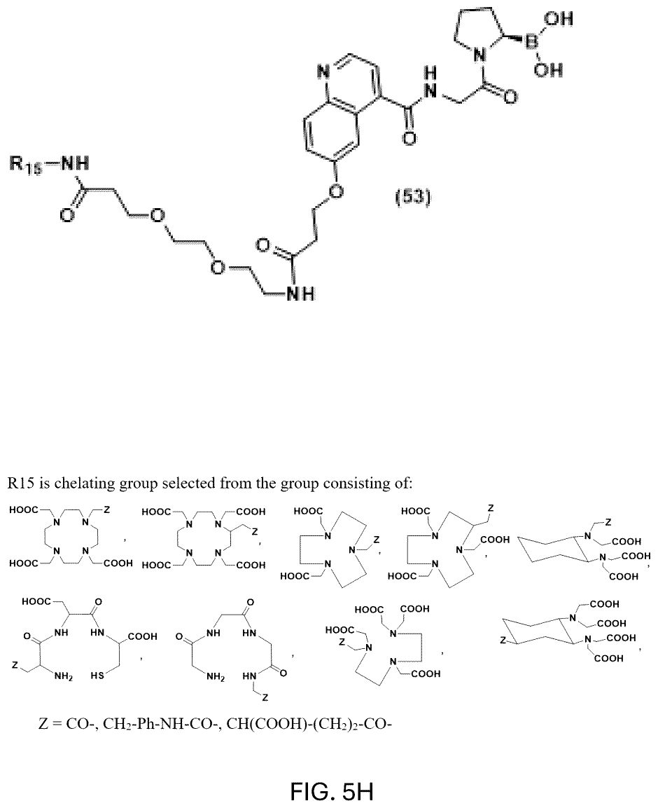

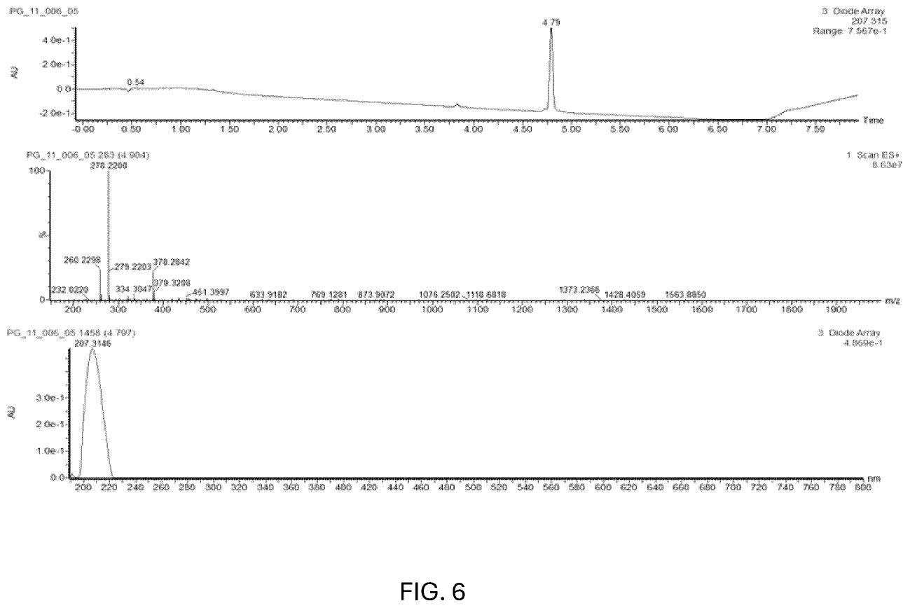

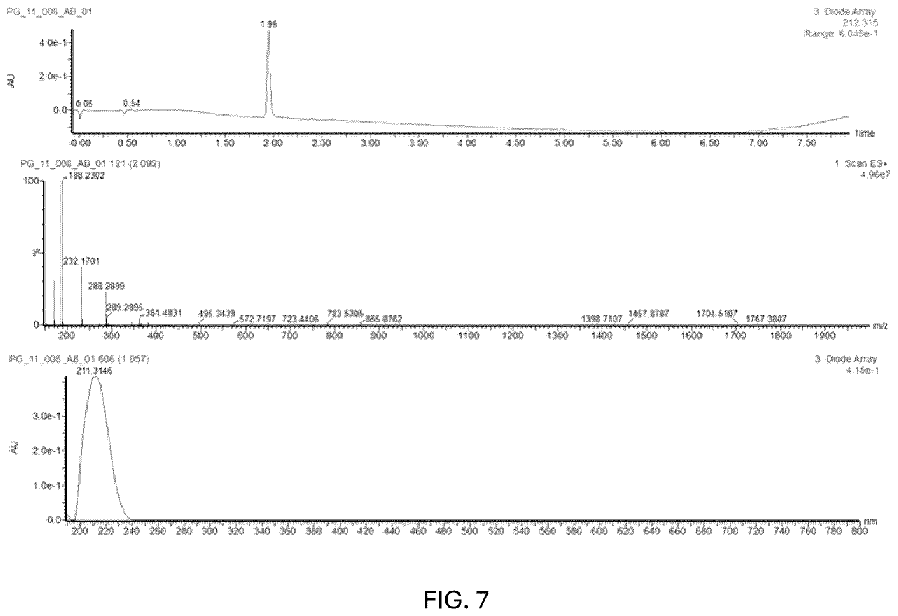

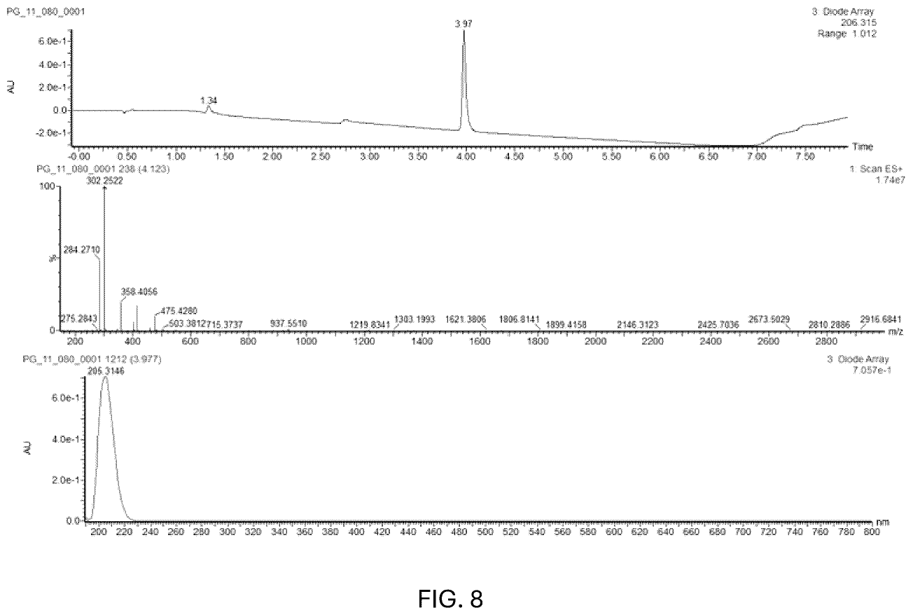

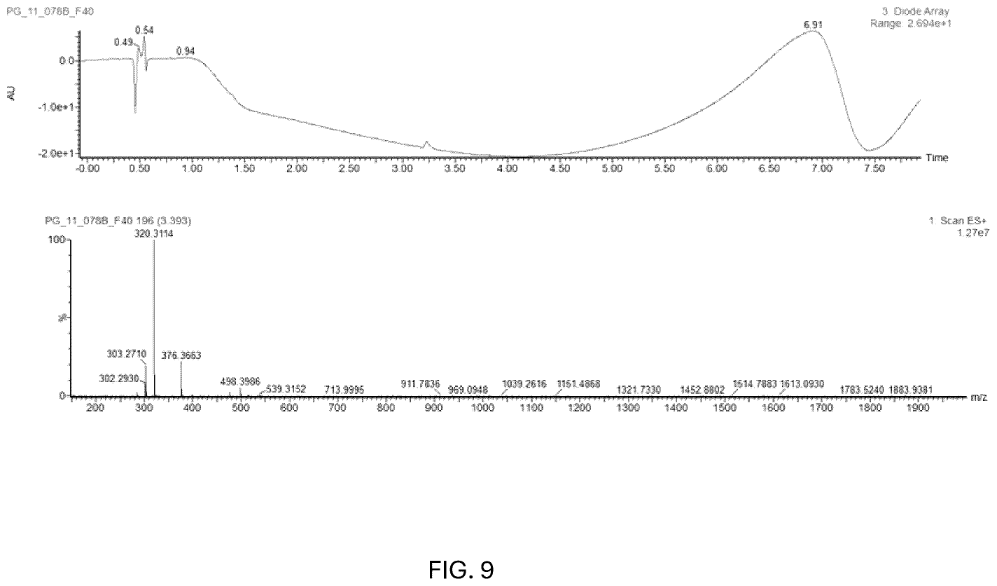

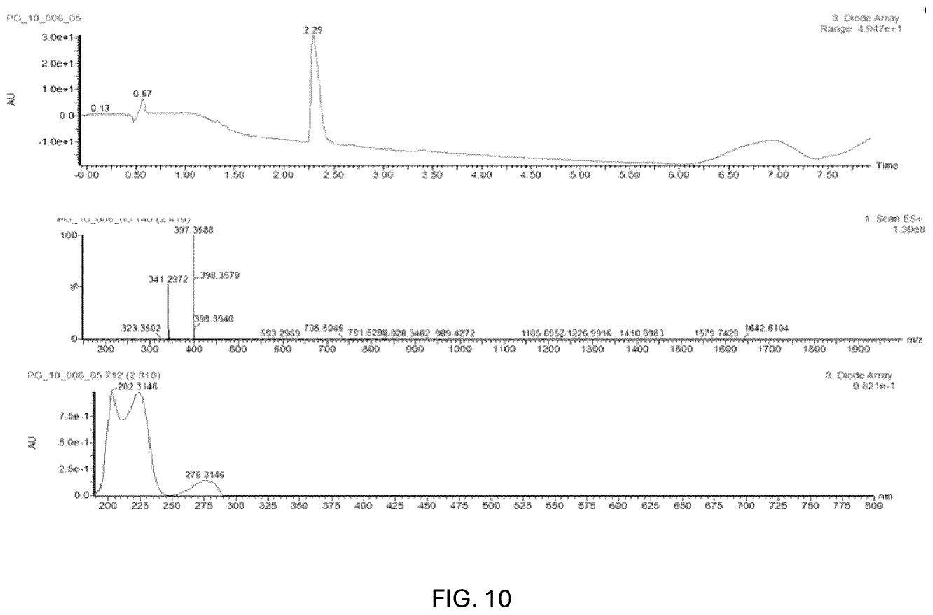

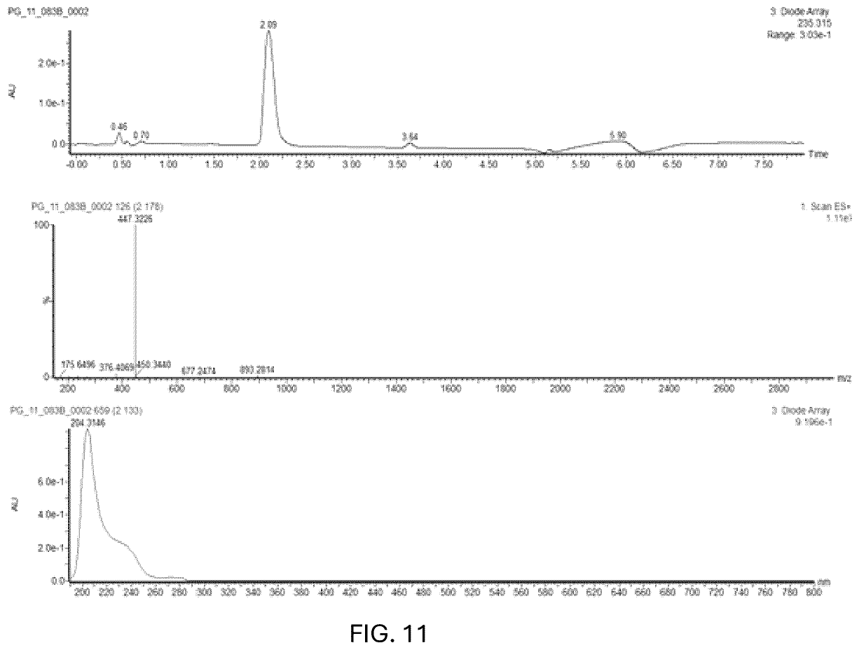

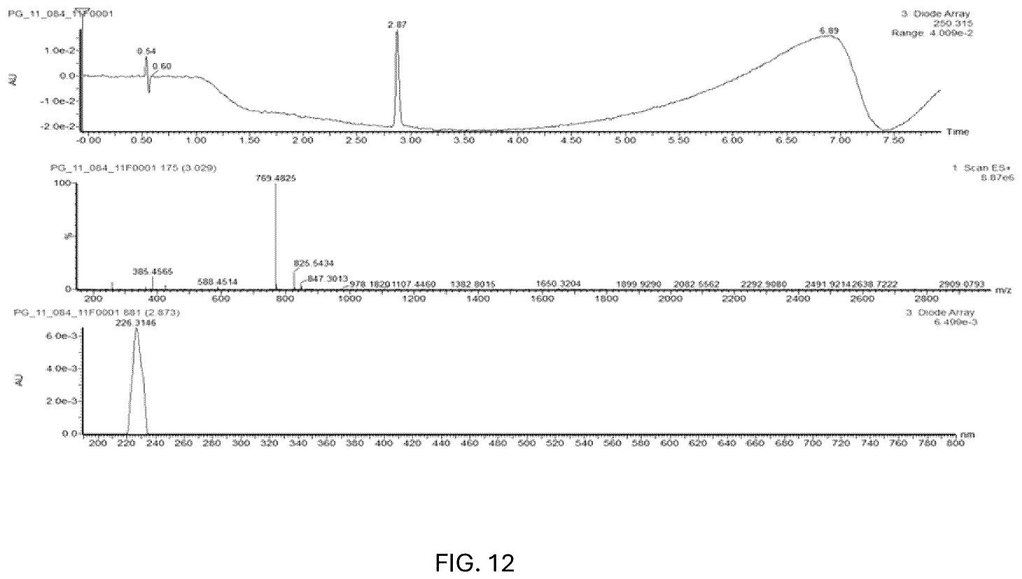

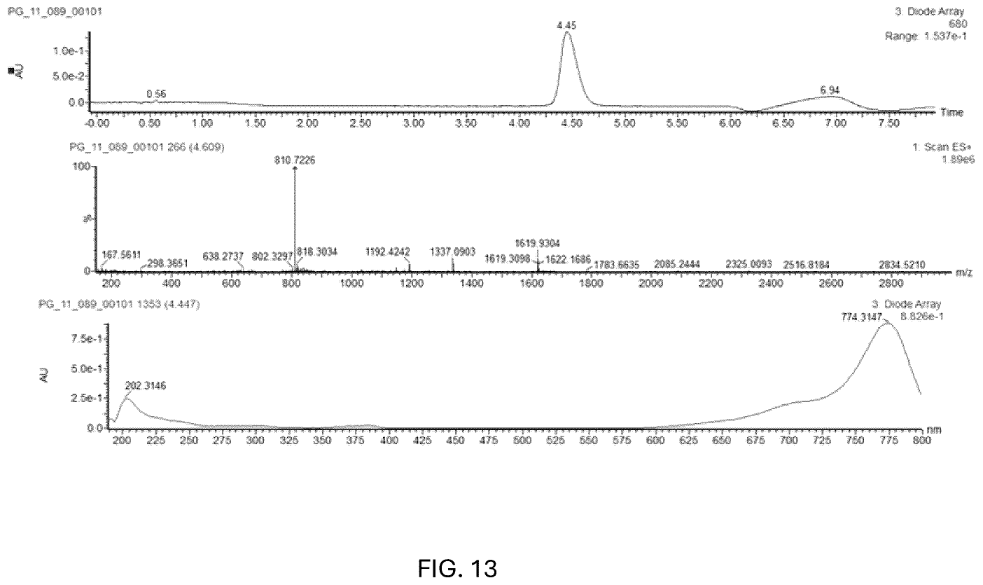

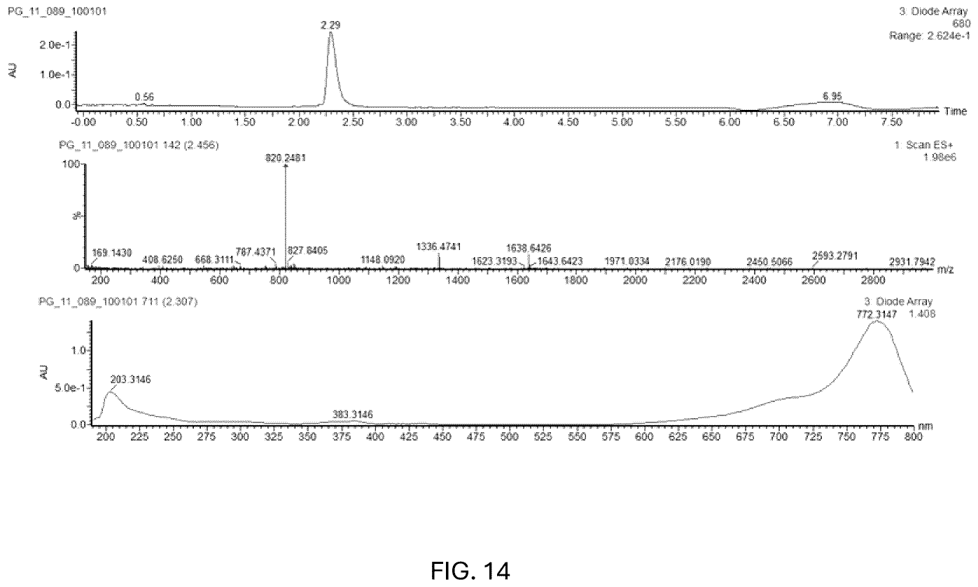

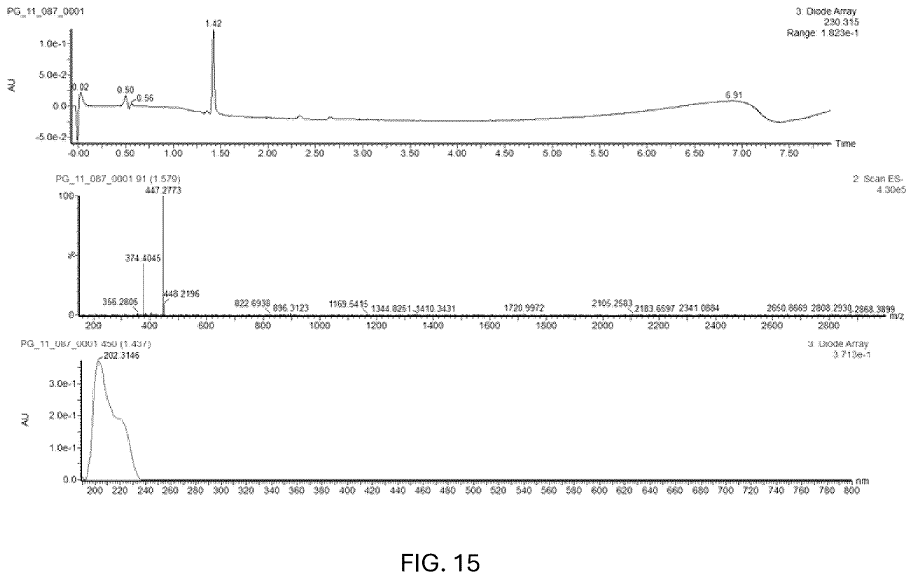

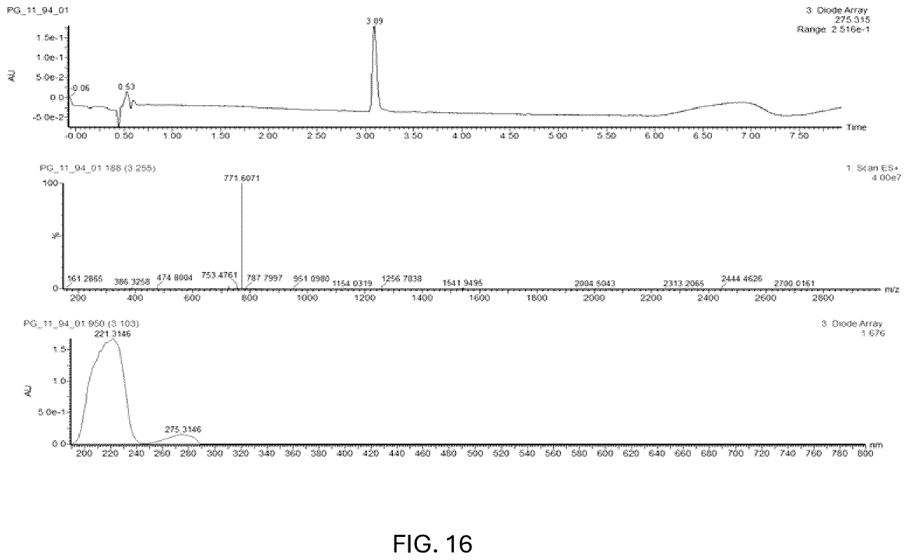

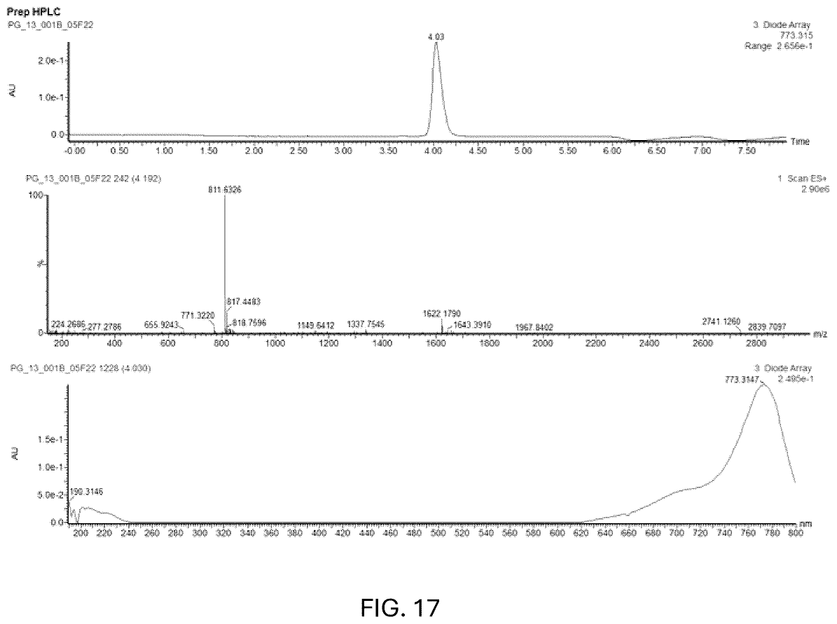

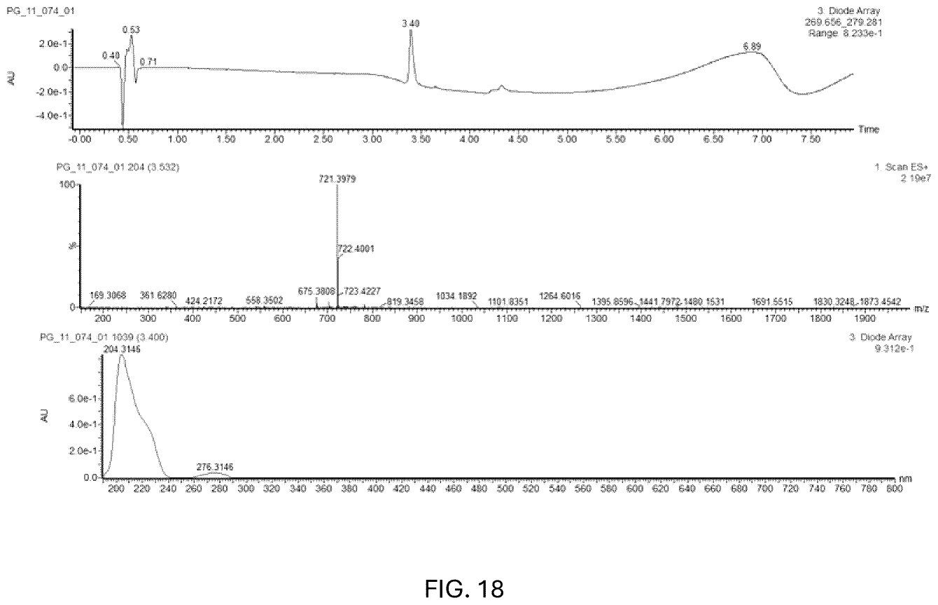

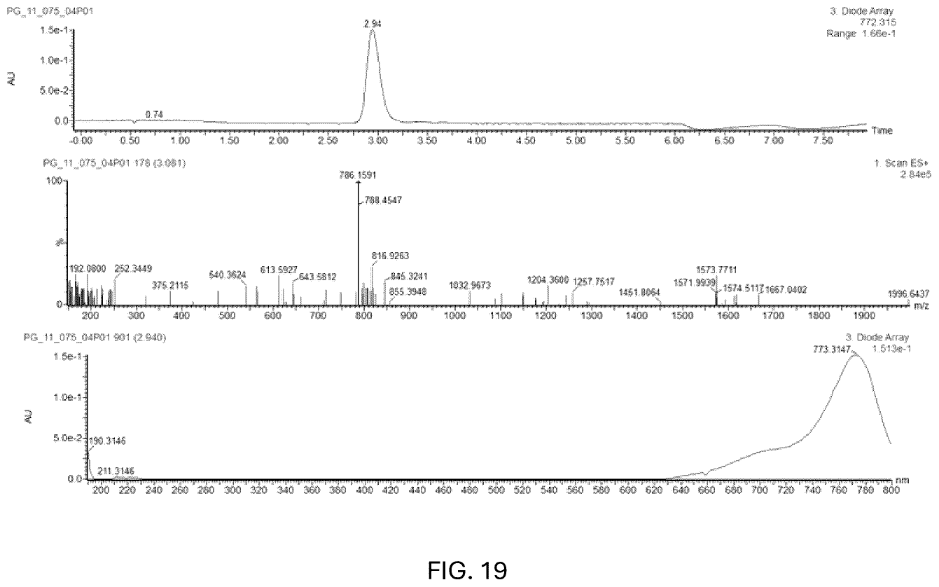

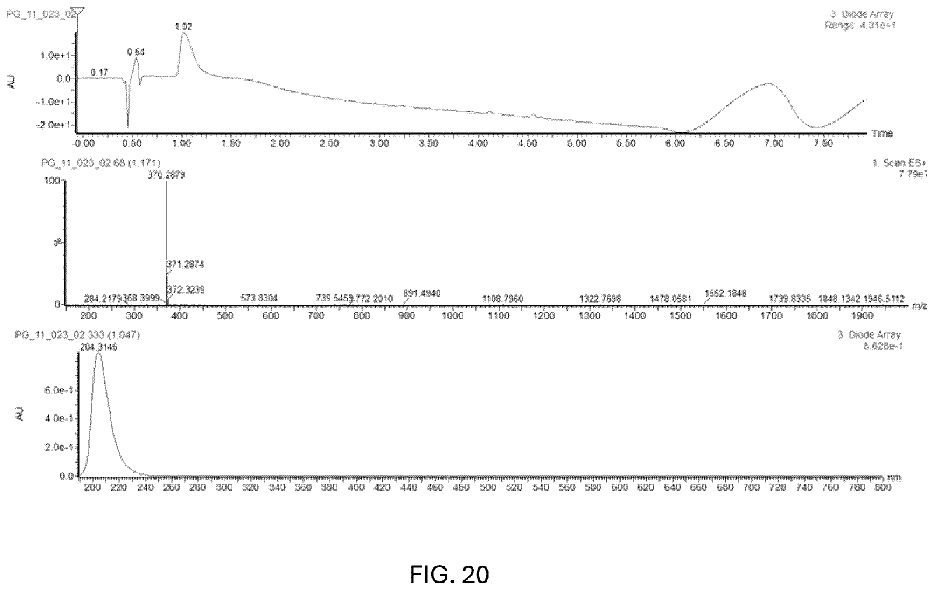

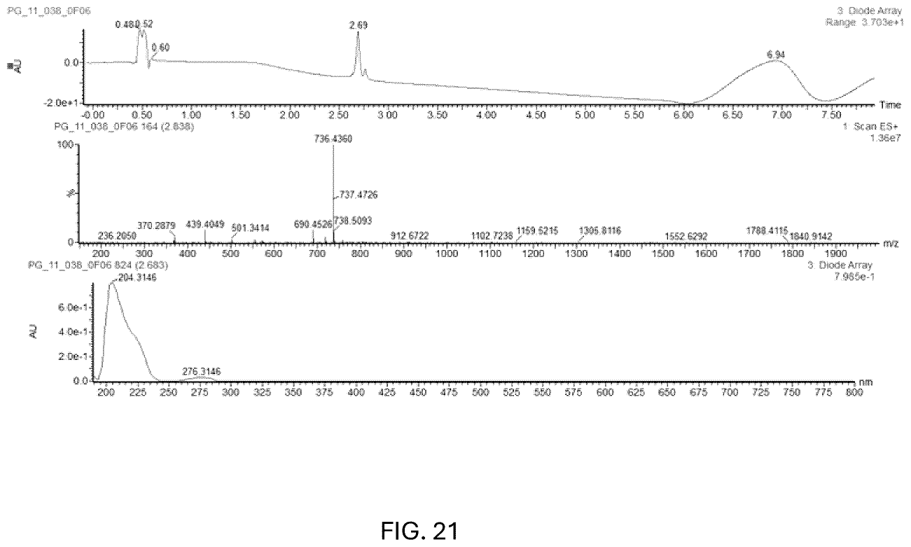

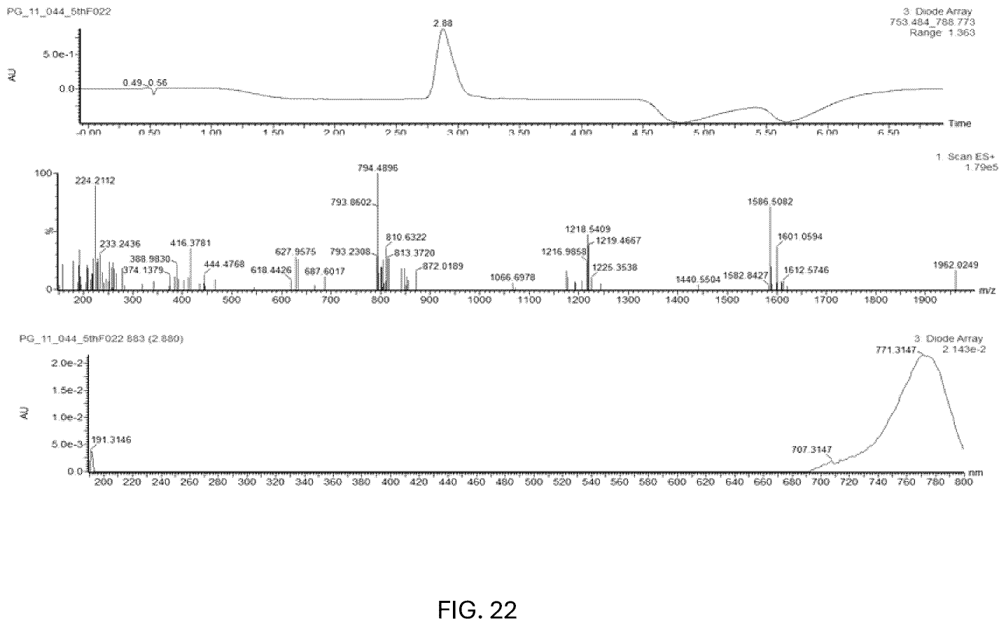

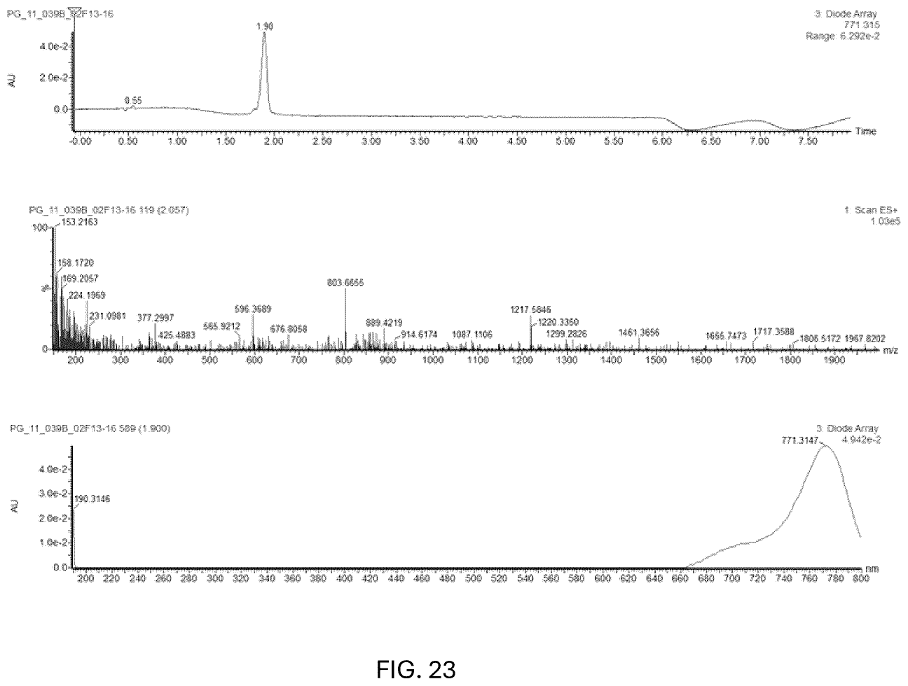

Other non-limiting examples of FAPI-image-guided surgery agents include Fibroblast Activating Protein Inhibitors with Cyanide (FAPI-Cyno) functional group-NIR dye conjugates (See FIGS. 1A-1C), Fibroblast Activating Protein Inhibitors with Amide (FAPI-Amido) functional group-NIR dye conjugates (See FIGS. 2A-2C), Fibroblast Activating Protein Inhibitors with α-Keto Amide (FAPI-KetoAmido) functional group-NIR dye conjugates (See FIGS. 3A-3C), Fibroblast Activating Protein Inhibitors with Boronic acid (FAPI-Boro) functional group-NIR dye conjugates (See FIGS. 4A-4C), and Fibroblast Activating Protein Inhibitors with cyno, amido, ketoamido, or Boro functional groups-radio conjugates (see FIGS. 5A-5H).

In some aspects, when the disclosed compounds are administered to a subject the compounds will fluoresce (or produce fluorescence) distribution thereof in the tissue cells of a subject. In an aspect, the disclosed compounds are made to fluoresce by subjecting the compounds to excitation light of near infrared wavelength.

### V. Methods of Use

Optical imaging is a non-invasive technique that is useful for visualizing tissues, including cancerous tissues and tumors. In some aspects, the disclosed compounds are used in optical imaging methods. In some embodiments, the compound improves the tumor-to-background ratio.

In an embodiments, a subject may be administered a compound (or a composition comprising said compound). The biological sample is then illuminated with an excitation light of a wavelength absorbable by the compound and the optical signal emitted by the compound is the detected. In some aspects, the biological sample is selected from the group consisting of urine, blood, oral fluid, plasma, tissue, bone marrow, and tumor samples. In some aspects, the biological sample expresses FAP. In another aspect, an area of the biological sample that expresses FAP is a tumor micro-environment. In another aspect, the biological sample comprises tumor cells and/or FAP-expressing cells.

As used herein, the terms “subject”, “individual”, and “patient” are interchangeable, and relate to vertebrates, preferably mammals. For example, mammals in the context of the disclosure are humans, non-human primates, domesticated animals such as dogs, cats, sheep, cattle, goats, pigs, horses, etc., laboratory animals such as mice, rats, rabbits, guinea pigs, etc., as well as animals in captivity such as animals in zoos. The term “animal” as used herein includes humans. The term “subject” may also include a patient, i.e., an animal, having a disease. In exemplary aspects, a subject, individual, or patient refers to a human (e.g., a man, a woman, or a child).

In an aspect, the biological sample is more than about 5 mm below the skin of the subject, alternatively more than about 6 mm below the skin of the subject, alternatively more than about 7 mm below the skin of the subject, alternatively more than about 8 mm below the skin of the subject, alternatively more than about 9 mm below the skin of the subject, or alternatively more than about 10 mm below the skin of the subject.

In some aspects, the composition comprising the compound is formulated for administration to a subject in need thereof. In yet another aspect, the composition further comprises a pharmaceutically acceptable carrier, excipient or diluent.

In some aspects, the methods include constructing an image from the optical signal emitted by the compound. For example, the optical signal detected and/or the image constructed can be used to detect the presence or absence of the compound of in the biological sample and/or diagnose a disease.

In another aspect, the method further comprises contacting the biological tissue with a fluorescent compound, wherein the fluorescent compound has a signal property distinguishable from the signal properties of the disclosed compounds.

Image guided surgery refers to any surgical procedure in which the surgeon utilizes tracked surgical instruments alongside preoperative or intraoperative images to guide the procedure directly or indirectly. Systems for image guided surgery may comprise cameras, ultrasonics, electromagnetics, or a combination of these technologies to capture and transmit a subject's anatomy and the surgeon's precise movements relative to the subject. This information may be displayed on computer monitors within the operating room or through augmented reality headsets (augmented reality surgical navigation technology). In some aspects, this guidance occurs in real-time.

One aspect of the disclosure includes a method for performing image guided surgery on a subject in need thereof. In an embodiment, the method includes administering a composition comprising a disclosed compound, illuminating the compound using an excitation light, and performing surgical resection of the areas that fluoresce upon excitation by the excitation light

In some aspects, the image guided surgery is radio-guided surgery. Radio-guided surgery comprises the use of radioactive imaging agents to in imaged surgery. In a non-limiting aspect, a radio-guided surgery may comprise administering a FAPI-radio-labelled tracer to a subject which is taken up by a tumor. A surgeon may then used a radiation detection probe to look for the FAP targeted tumors.

In some aspects, the image guided surgery is fluorescence image guided surgery. Fluorescence image guided surgery comprises the use of imaging devices to provide real-time simultaneous information from color reflectance images (bright field) and fluorescence emission. In some embodiments, one or more light sources are employed to excite and illuminate a sample, with optical filters used to collect light matching the emission spectrum of the fluorophore. Imaging lenses and digital cameras (CCD or CMOS) are utilized to capture the final image. Live video processing can enhance contrast during fluorescence detection and improve the signal-to-background ratio.

Fluorescence excitation employs various light sources. In some aspects, the light source is a halogen lamp, light-emitting diode (LED), or a laser. In other aspects, the light source may comprise different band-pass filters, enabling it to produce several excitation channels from ultraviolet (UV) to near-infrared (NIR). LEDs are commonly used for broad-band illumination and narrow-band excitation in FGS. LEDs emit light within a narrow wavelength range that matches the absorption spectrum of a specific fluorophore, reducing the complexity of the optical system. Laser diodes are used when high power over a short wavelength range is needed, requiring consideration of exposure limits to laser radiation.

In an aspect, the excitation light is a near-infrared wavelength light or infrared light. In another aspect, the excitation light has a wavelength range from about 600 nm to about 1000 nm, alternatively about 670 nm to about 850 nm, alternatively between about 650 nm to about 900 nm, alternatively between about 650 nm to about 1000 nm, alternatively about 800 nm.

Live images of fluorescent dye and the surgical field are obtained using a combination of filters, lenses, and cameras. Fluorescence image guided surgery may also be performed using minimally invasive devices like laparoscopes or endoscopes, where a system of filters, lenses, and cameras is attached to the probe. Fluorescence image guided surgery may be performed using robotic surgery setups or handheld devices. In some aspects of the method, an endoscope, catheter, tomographic system, hand-held optical imaging system, surgical goggles, or intra-operative microscope is in any of the disclosed methods. In an aspect, the use of the compound improves surgical resection and/or provides cleaner surgical margins than non-NIR conjugated fluorescing dyes. In another aspect, the areas that fluoresce upon excitation are tumor micro-environments or comprise tumor cells.

The optical imaging and/or image guided surgery methods may be used to image tumors. The optical imaging methods may be used to image tumors in vivo, in vitro, or ex vivo. In some aspects, the volume of the tumor is at least 1000 mm³. In some aspects, the volume of the tumor is less than 1000 mm³. In some aspects, the volume of the tumor is less than 950 mm³. In some aspects, the volume of the tumor is less than 900 mm³. In some aspects, the volume of the tumor is less than 850 mm³. In some aspects, the volume of the tumor is less than 800 mm³. In some aspects, the volume of the tumor is less than 750 mm³. In some aspects, the volume of the tumor is less than 700 mm³. In some aspects, the volume of the tumor is less than 650 mm³. In some aspects, the volume of the tumor is less than 600 mm³. In some aspects, the volume of the tumor is less than 550 mm³. In some aspects, the volume of the tumor is less than 500 mm³. In some aspects, the volume of the tumor is less than 450 mm³. In some aspects, the volume of the tumor is less than 400 mm³. In some aspects, the volume of the tumor is less than 350 mm³. In some aspects, the volume of the tumor is less than 300 mm³. In some aspects, the volume of the tumor is less than 250 mm³. In some aspects, the volume of the tumor is less than 200 mm³. In some aspects, the volume of the tumor is less than 150 mm³. In some aspects, the volume of the tumor is less than 100 mm³. In one aspect, the volume of the tumor is at least 75 mm³. In another aspect, the volume of the tumor is less than 75 mm³. In another aspect, the volume of the tumor is less than 70 mm³. In another aspect, the volume of the tumor is less than 65 mm³. In another aspect, the volume of the tumor is less than 60 mm³. In another aspect, the volume of the tumor is less than 55 mm³. In one aspect, the volume of the tumor is at least 50 mm³. In other aspects, the tumor is less than 50 mm³. In another aspect, the volume of the tumor is less than 45 mm³. In other aspects, the volume of the tumor is less than 40 mm³. In another aspect, the volume of the tumor is less than 35 mm³. In still another aspect, the volume of the tumor is less than 30 mm³. In another aspect, the volume of the tumor is less than 25 mm³. In still another aspect, the volume of the tumor is less than 20 mm³. In another aspect, the volume of the tumor is less than 15 mm³. In still another aspect, the volume of the tumor is less than 10 mm³. In still another aspect, the volume of the tumor is less than 12 mm³. In still another aspect, the volume of the tumor is less than 9 mm³. In still another aspect, the volume of the tumor is less than 8 mm³. In still another aspect, the volume of the tumor is less than 7 mm³. In still another aspect, the volume of the tumor is less than 6 mm³. In still another aspect, the volume of the tumor is less than 5 mm³.

In one aspect, the tumor has a length of at least 5 mm prior to surgical recession using a compound. In one aspect, these methods detect tumors less than 5 mm. In other aspects the methods herein detect tumors less than 4 mm. In some aspects, the methods herein detect tumors less than 3 mm. In another aspect, the tumor has a length of at least 6 mm. In still another aspect, the tumor has a length of at least 7 mm. In yet another aspect, the tumor has a length of at least 8 mm. In another aspect, the tumor has a length of at least 9 mm. In still another aspect, the tumor has a length of at least 10 mm. In yet another aspect, the tumor has a length of at least 11 mm. In a further aspect, the tumor has a length of at least 12 mm. In still a further aspect, the tumor has a length of at least 13 mm. In still a further aspect, the tumor has a length of at least 14 mm. In another aspect, the tumor has a length of at least 15 mm. In yet another aspect, the tumor has a length of at least 16 mm. In still another aspect, the tumor has a length of at least 17 mm. In a further aspect, the tumor has a length of at least 18 mm. In yet a further aspect, the tumor has a length of at least 19 mm. In still a further aspect, the tumor has a length of at least 20 mm. In another aspect, the tumor has a length of at least 21 mm. In still another aspect, the tumor has a length of at least 22 mm. In yet another aspect, the tumor has a length of at least 23 mm. In a further aspect, the tumor has a length of at least 24 mm. In still a further aspect, the tumor has a length of at least 25 mm. In yet a further aspect, the tumor has a length of at least 30 mm.

The optical imaging and/or image guided surgery methods may be used to image FAP-expressing cells. The optical imaging methods may be used to image tumors in vivo, in vitro, or ex vivo.

An aspect of the disclosure includes a method of diagnosing a disease in a subject in need thereof, the method comprising: administering a composition comprising a disclosed compound; illuminating the compound using an excitation light; detecting the optical signal emitted by the compound; comparing the signal detected with at least one control data set, wherein the at least one control data set comprises signals from the compound contacted with a biological sample that does not comprise a target cell type; and providing a diagnosis of disease wherein the comparison in indicates the presence of the disease.

A “disease”, as used herein, is a state of health of a subject wherein the subject cannot maintain homeostasis, and wherein if the disease is not ameliorated, the subject's health continues to deteriorate. In contrast, a “disorder” is a state of health in which the subject is able to maintain homeostasis, but in which the subject's state of health is less favorable than it would be in the absence of the disorder. Left untreated, a disorder does not necessarily cause a further decrease in the subject's state of health. A disease or disorder is “alleviated” if the severity of a sign or symptom of the disease or disorder, the frequency with which such a sign or symptom is experienced by a subject, or both, is reduced.

In an aspect, the FAP-expressing cells are benign or non-cancer cells. In another aspect, the FAP-expressing cells are malignant or cancer cells.

In some aspects, the disclosed methods are used to detect cells with high FAP expression or FAP overexpression. In some aspects, the cells are selected from the group consisting of prostate cells, prostate cancer cells, bladder cancer cells, pancreatic cancer cells, liver cancer cells, lung cancer cells, kidney cancer cells, sarcoma cells, breast cancer cells, brain cancer cells, neuroendocrine carcinoma cells, colon cancer cells, testicular cancer cells and melanoma cells.

In some aspects, the disclosed methods are used to detect cancer cells. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid, uterus, vagina, and vulva.

In some aspects, the disclosed methods are used to treat cancer. In some aspects, the cancer is selected from the group consisting of prostate cancer, lung cancer, bladder cancer, pancreatic cancer, liver cancer, kidney cancer, sarcoma, breast cancer, brain cancer, neuroendocrine carcinoma, colon cancer, testicular cancer or melanoma.

The terms “treat”, “treating”, or “treatment” refer to administering to a subject a compound or pharmaceutical composition disclosed herein to partially or completely alleviate, inhibit, ameliorate, or relieve the disease or disorder from which the subject is suffering. This means any manner in which one or more of the symptoms of a disease or disorder are ameliorated or otherwise beneficially altered. As used herein, amelioration of the symptoms of a particular disease or disorder refers to any lessening, whether permanent or temporary, lasting or transient, that can be attributed to or associated with treatment by the compounds, compositions, and methods of the present disclosure. For example, treating a subject can mean eliminating or reducing the clinical signs of a disease or disorder in the subject; arrest, inhibit, or slow the progression of the disease or disorder in the subject; and/or decrease the number, frequency, or severity of clinical symptoms and/or recurrence of the disease or disorder in the subject who currently has or who previously had the disease or disorder. In particular, the terms “treatment of a disease” and “treating a disease” include curing, shortening in duration, ameliorating, slowing down, inhibiting progression or worsening, or delaying the onset of clinical symptoms in a subject who has the disease or disorder.

In some embodiments, the disclosed compounds may include a therapeutic agent. In some embodiments, the compounds are therapeutically effective against cancer cells and/or cancer-associated fibroblast (CAFs). The therapeutic agent used may be any molecule capable of modulating or otherwise modifying cell function, including pharmaceutically active compounds.

Suitable molecules that may be useful as therapeutic agents include but are not limited to peptides, oligopeptides, retro-inverso oligopeptides, proteins, protein analogs in which at least one non-peptide linkage replaces a peptide linkage, apoproteins, glycoproteins, enzymes, coenzymes, enzyme inhibitors, amino acids and their derivatives, receptors and other membrane proteins; antigens and antibodies thereto; haptens and antibodies thereto; hormones, lipids, phospholipids, liposomes; toxins; antibiotics; analgesics; bronchodilators; beta-blockers; antimicrobial agents; antihypertensive agents; cardiovascular agents including antiarrhythmics, cardiac glycosides, antianginals and vasodilators; central nervous system agents including stimulants, psychotropics, antimanics, and depressants; antiviral agents; antihistamines; cancer drugs including chemotherapeutic agents; tranquilizers; anti-depressants; H-2 antagonists; anticonvulsants; antinauscants; prostaglandins and prostaglandin analogs; muscle relaxants; anti-inflammatory substances; stimulants; decongestants; antiemetics; diuretics; antispasmodics; antiasthmatics; anti-Parkinson agents; expectorants; cough suppressants; mucolytics; and mineral and nutritional additives.

In another aspect, methods for treating diseases and disease states, diagnosing diseases or disease states, and/or imaging tissues and/or cells that are associated with pathogenic populations of cells expressing or over expressing FAP are described herein. Such methods include the step of administering the conjugates described herein, and/or pharmaceutical compositions containing the conjugates described herein, in amounts effective to treat diseases and disease states, diagnose diseases or disease states, and/or image tissues and/or cells that are associated with pathogenic populations of cells expressing or over expressing FAP.

Non-limiting aspects of the invention are further described in the following paragraphs:
1. A compound having a formula B—X—Z, wherein
   B comprises a Fibroblast Activating Protein (FAP)-targeted molecule;
   X comprises a spacer; and
   Z comprises a NIR dye.
2. The compound of subparagraph 1, wherein B is a monovalent group derived from a FAP-targeted molecule.
3. The compound of subparagraph 1 or subparagraph 2, wherein B is selected from the group consisting of a small molecule, a ligand, an inhibitor, an agonist, and a derivative thereof.
4. The compound of subparagraph 3, wherein the small molecule binds FAP.
5. The compound of subparagraph 3 or subparagraph 4, wherein the small molecule comprises an extended hydrophobic moiety with alpha keto amide unit, a substituted alpha keto amide, or a substituted alpha keto amide with aromatic moiety with methoxy units.
6. The compound of any one of the preceding subparagraphs, wherein X is a hydrophobic spacer.
7. The compound of any one of the preceding subparagraphs, wherein X is variably charged, has a negative charge, or has a positive charge.
8. The compound of any one of the preceding subparagraphs, wherein X is selected from the group consisting of six aminohectanoic acid (SAHA), eight aminooctonoic acid (EAOA), polyethylene glycol (PEG), polyethylene amine (PEA) unit, and N-amino-dPEG₂ acid.
9. The compound of any one subparagraphs 1-7, wherein X is a peptide comprising at least one aryl group or at least one aryl alkyl group, each of which is optionally substituted.
10. The compound of subparagraph 9, wherein the peptide comprises at least two aryl or aryl alkyl group, wherein a first aryl or aryl alkyl group is about 6 atoms to about 10 atoms, alternatively about 6 to about 14 atoms, and a second aryl or aryl alkyl group is about 10 atoms to about 14 atoms, alternatively about 10 atoms to about 15 atoms.
11. The compound of any one of subparagraphs 1-7, wherein X is a peptide comprising at least one amino acid selected from the group consisting of a quaternary amine containing amino acid, an acidic amino acid, a basic amino acid, a neutral polar amino acid, a neutral nonpolar amino acid, an aromatic amino acid, and amino acid derivatives.
12. The compound of subparagraph 11, wherein the acidic amino acid is selected from the group consisting of aspartic acid and glutamic acid.
13. The compound of subparagraph 11, wherein the basic amino acid is selected from the group consisting of arginine, lysine, histidine, and ornithine.
14. The compound of subparagraph 11, wherein the neutral polar amino acid is selected from the group consisting of glycine, serine, threonine, cysteine, tyrosine, asparagine, and glutamine.
15. The compound of subparagraph 11, wherein the neutral nonpolar amino acid is selected from the group consisting of alanine, leucine, isoleucine, valine, proline, phenylalanine, tryptophan, and methionine.
16. The compound of subparagraph 11, wherein the amino acid derivative is derived from tyrosine.
17. The compound of subparagraph 16, wherein the amino acid derivative is tyramine.
18. The compound of subparagraph 16, wherein the amino acid derivative is selected from the group consisting of:

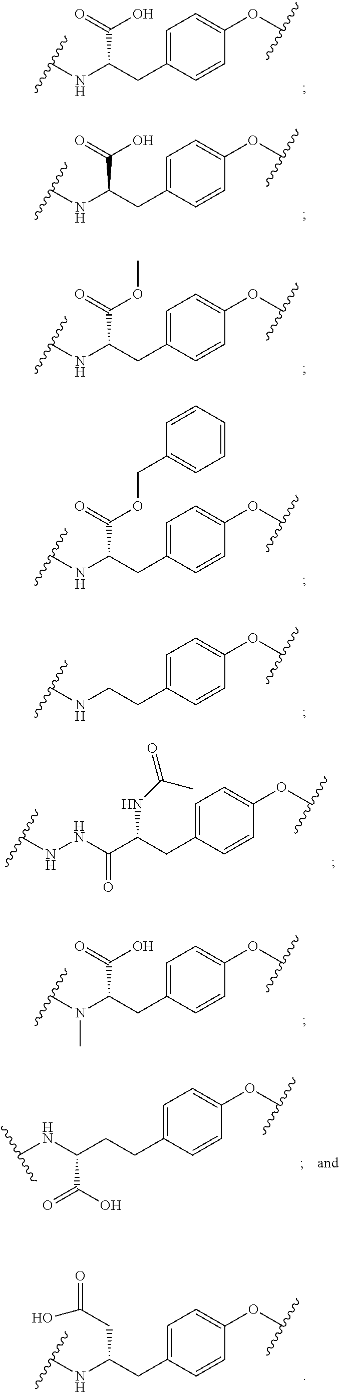

19. The compound of any one of subparagraphs 1-7, wherein X comprises an amino acid spacer with sulfur-containing side chain group.
20. The compound of subparagraph 19, wherein the sulfur-containing side group is selected from the group consisting of cysteine, methionine and a molecule containing a thiophenol moiety.
21. The compound of any one of subparagraphs 1-7, wherein X comprises an amino acid spacer with a chalcogen-containing side chain group.
22. The compound of any one of subparagraphs 1-7, wherein X comprises a single amino acid selected from the group consisting of tyrosine, cysteine, lysine, tyramine, a tyrosine derivative, a cysteine derivative, and a lysine derivative.
23. The compound of subparagraph 22, wherein the tyrosine derivative is selected from the group consisting of:

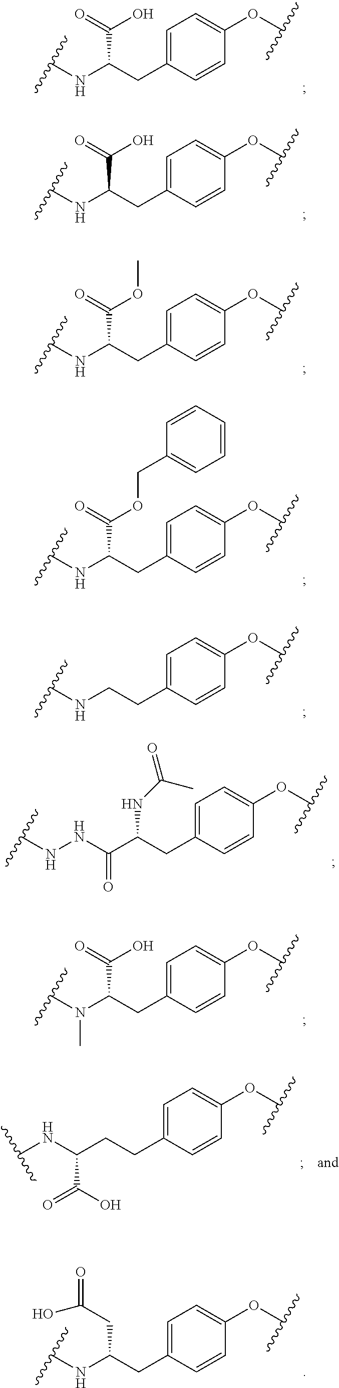

24. The compound of any one of subparagraphs 1-7, wherein X comprises an amino acid isotope.
25. The compound of subparagraph 24, wherein the amino acid is tyrosine, wherein the tyrosine comprises a hydrogen isotope or carbon isotope on the aromatic ring.
26. The compound of any one the preceding subparagraphs, wherein X has a length of about 1 atoms to about 20 atoms, alternatively about 2 atoms, alternatively about 3 atoms, alternatively about 4 atoms, alternatively about 5 atoms, alternatively about 6 atoms, alternatively about 7 atoms, alternatively about 8 atoms, alternatively about 9 atoms, alternatively about 10 atoms, alternatively about 11 atoms, alternatively about 12 atoms, alternatively about 13 atoms, alternatively about 14 atoms, alternatively about 15 atoms, alternatively about 16 atoms, alternatively about 17 atoms, alternatively about 18 atoms, alternatively about 19 atoms, alternatively about 20 atoms.
27. The compound of any one of the preceding subparagraphs, wherein Z comprises a monovalent group derived from a NIR dye.
28. The compound of any one of the preceding subparagraphs, wherein the NIR dye is selected from the group consisting of:

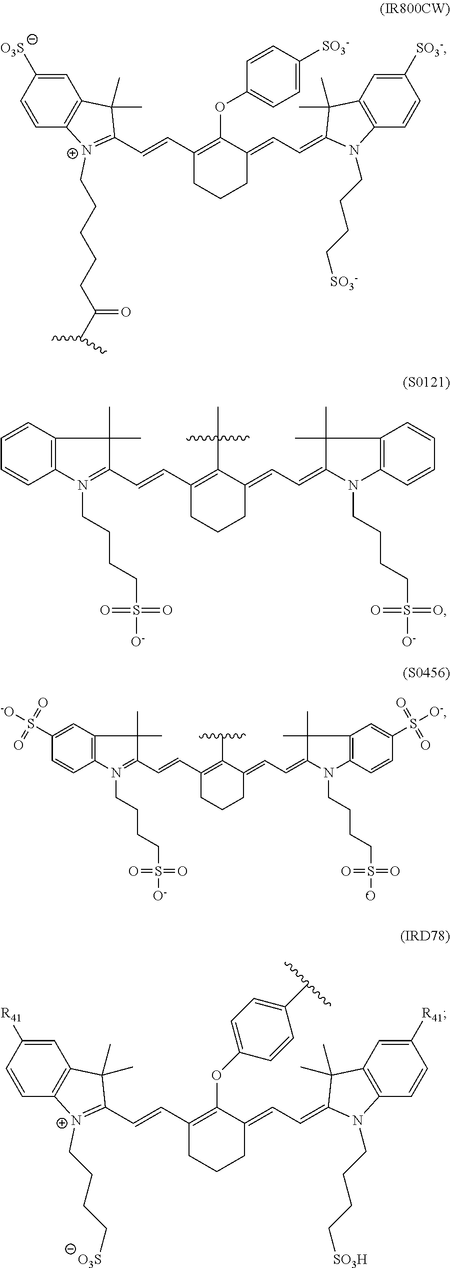

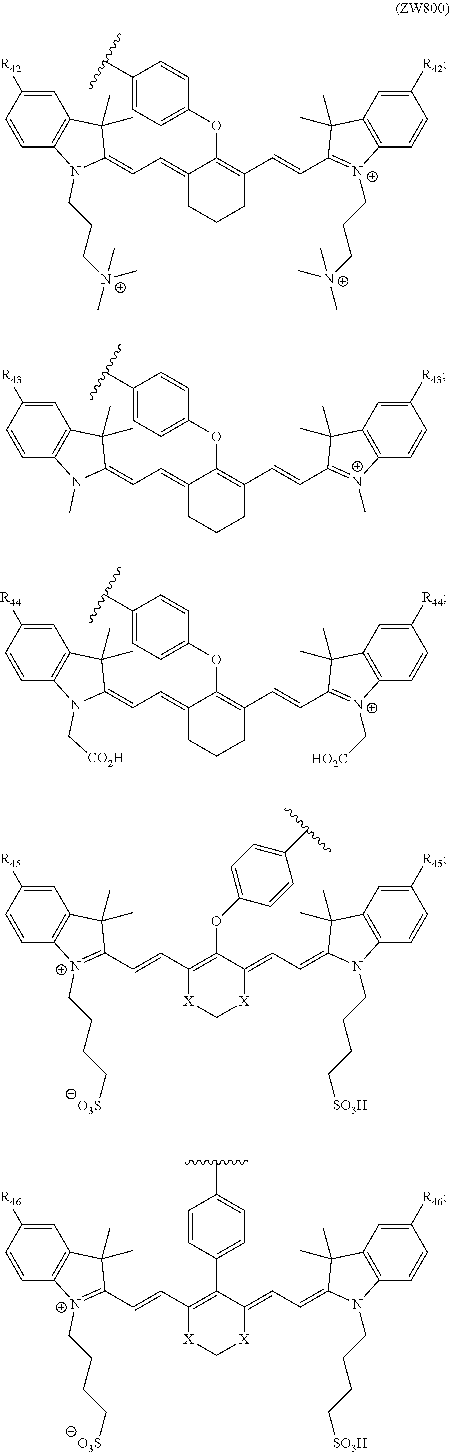

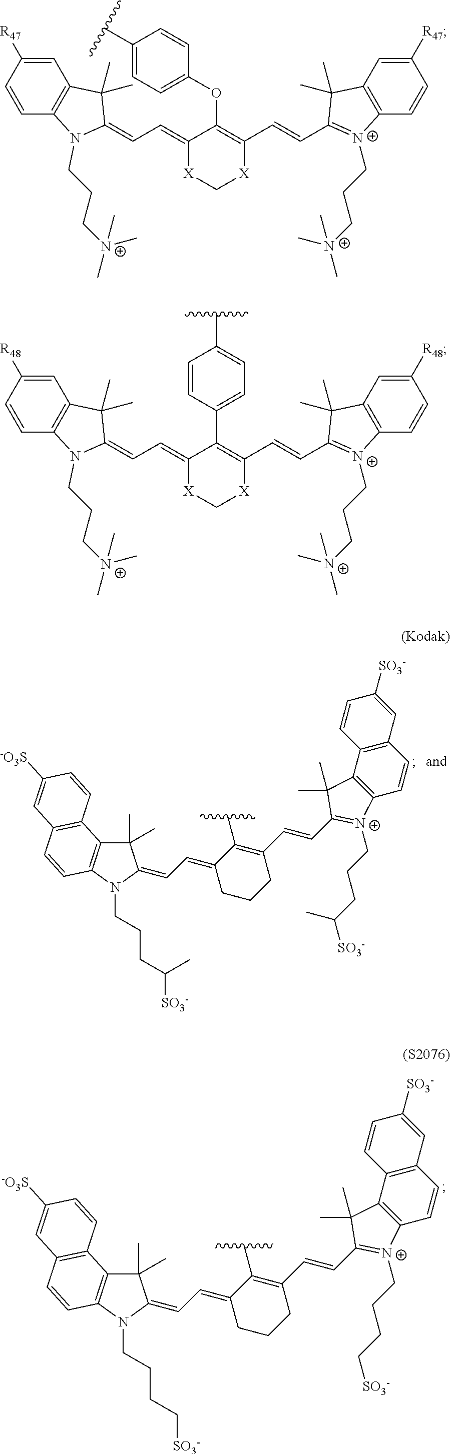

wherein R41, R42, R43, R44, R45, R46, R47, R48=H or SO₃H; X═O, S, or N.
29. The compound of subparagraph 28, wherein B comprises a small molecule or a ligand, X comprises eight aminooctonoic acid (EAOA) or polyethylene glycol (PEG), and Z comprises IRD78.
30. The compound of any one of the preceding subparagraphs, wherein Z is variably charged, has a negative charge, or has a positive charge.
31. A compound having a formula:

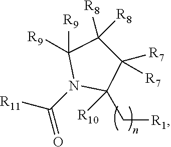

wherein R¹ is CHO, C═CH, CH(CN)₂, CN, NHCN, B(OH)R₂, COR₂, Zn(OH), PO(OH)R₂, SO₂R₂, Si(OH)₂R₂, a halogen, OH, H or COCONHR₃;
wherein R₂ is OH, SH, NH₂, O-alkyl, S-alkyl, NH-alkyl, a halogen, or H; and
R₃ is H, a halogen, OH, methyl, ethyls, isopropyl, tBu, cyclopropyl, alkyl, alkyl-OH, CN, CHPh₂, adamantane or

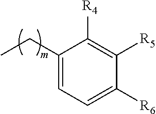

wherein R₄, R₅, or R₆ are independently selected from the group consisting of H, OCH₃, SCH₃, COR₂, SO₂R₂, PO(OH)R₂, Si(OH)₂R₂, halogen, OH, CN, B(OH)R₂, and combinations thereof;
R₇, R₈, R₉, or R₁₀ are independently selected from the group consisting of H, a halogen, OH, OCH₃, SCH₃, CN, COR₂, SO₂R₂, PO(OH)R₂, Si(OH)₂R₂, B(OH)R₂, and combinations thereof;
n and m are independently selected from 0, 1, 2, 3, 4, or 5;
R₁₁ is selected from the group consisting of

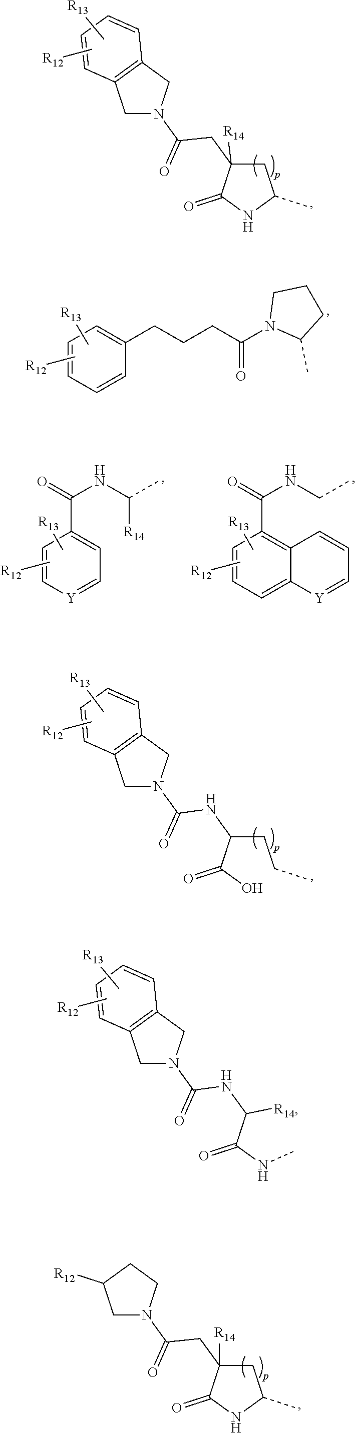

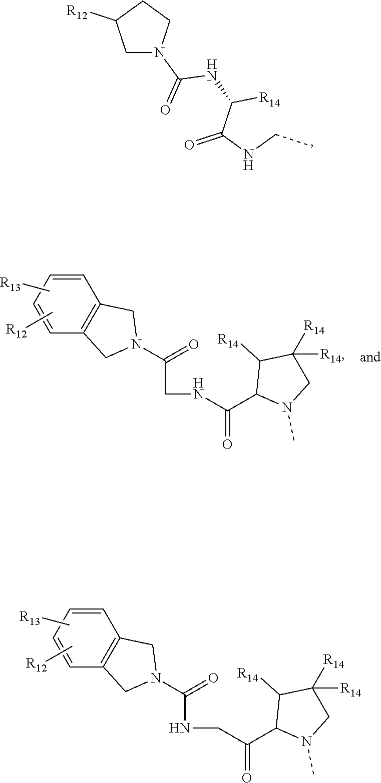

wherein R₁₂ is (CH₂)q-X, X=COOH, SH, OH, NH₂, halogen, or maleimide;
R₁₃ is H, halogen, Me, OH, SH, OCH₃, OBn, or CH₂OH;
R₁₄ is H, F, CH₃, OCH₃, Isopropyl, or Tert-butyl;
Y is CH or N; and
p and q are independently selected from 0, 1, 2, 3, 4, or 5.
32. The compound of any one of the preceding subparagraphs, wherein the compound has an absorption and emission maxima between about 500 nm and about 900 nm, alternatively between about 600 nm and about 800 nm, alternatively between about 650 nm and about 900 nm, alternatively between about 600 nm and about 1000 nm, alternatively about 800 nm.
33. The compound of any one of the preceding subparagraphs, wherein the compounds are made to fluoresce after distribution thereof in the tissue cells.
34. The compound of subparagraph 33, wherein the compounds are made to fluoresce by subjecting the compounds to excitation light of near infrared wavelength.
35. The compound of any one of the preceding subparagraphs, wherein the compound is highly selective for targeting to a tumor cell.
36. The compound of any one of the preceding subparagraphs, wherein the compound is targeted to breast, colorectal, pancreatic, lung, brain, intrahepatic bile duct, and/or ovarian cancer cells.
37. A composition comprising the compound of any one of the preceding subparagraphs.
38. The composition of subparagraph 37, wherein the composition is formulated for administration to a subject in need thereof.
39. The composition of subparagraph 37 or subparagraph 38, wherein the composition further comprises a pharmaceutically acceptable carrier, excipient or diluent.
40. A method of optical imaging of a biological sample, said method comprising:
   contacting the biological sample with a composition comprising a compound of any one of subparagraphs 1-36;
   illuminating the compound with an excitation light of a wavelength absorbable by the compound; and
   detecting the optical signal emitted by the compound.
41. The method of subparagraph 40, wherein the optical imaging occurs in a subject in need thereof.
42. The method of subparagraph 40 or subparagraph 41, wherein the biological sample is selected from the group consisting of urine, blood, oral fluid, plasma, tissue, bone marrow, and tumor samples.
43. The method of any one of subparagraphs 40-42, wherein the biological sample expresses FAP.
44. The method of subparagraph 43, wherein an area of the biological sample that expresses FAP is a tumor micro-environment.
45. The method of any one of subparagraphs 40-44, wherein the biological sample comprises tumor cells and/or FAP-expressing cells.
46. The method of any one of subparagraphs 42-45, wherein the volume of the tumor is at least 1000 mm³.
47. The method of any one of subparagraphs 42-45, wherein the volume of the tumor is less than 1000 mm³, alternatively less than 950 mm³, alternatively less than 900 mm³, alternatively less than 850 mm³, alternatively less than 800 mm³, alternatively less than 750 mm³, alternatively less than 700 mm³, alternatively less than 650 mm³, alternatively less than 600 mm³, alternatively less than 550 mm³, alternatively less than 500 mm³, alternatively less than 450 mm³, alternatively less than 400 mm³, alternatively less than 350 mm³, alternatively less than 300 mm³, alternatively less than 250 mm³, alternatively is less than 200 mm³, alternatively is less than 150 mm³, alternatively is less than 100 mm³, alternatively at least 75 mm³, alternatively less than 75 mm³, alternatively less than 70 mm³, alternatively less than 65 mm³, alternatively less than 60 mm³, alternatively less than 55 mm³, alternatively at least 50 mm³, alternatively less than 50 mm³, alternatively less than 45 mm³, alternatively less than 40 mm³, alternatively less than 35 mm³, alternatively less than 30 mm³, alternatively less than 25 mm³, alternatively less than 20 mm³, alternatively less than 15 mm³, alternatively less than 10 mm³, alternatively less than 12 mm³, alternatively less than 9 mm³, alternatively less than 8 mm³, alternatively less than 7 mm³, alternatively less than 6 mm³, alternatively less than 5 mm³.
48. The method of subparagraph 45, wherein the FAP-expressing cells are benign or non-cancer cells.
49. The method of subparagraph 45, wherein the FAP-expressing cells are malignant or cancer cells.
50. The method of any one of subparagraphs 40-49, wherein the method is used to detect cells with high FAP expression or FAP overexpression.
51. The method of any one of subparagraph 45-50, wherein the cells are selected from the group consisting of prostate cells, prostate cancer cells, bladder cancer cells, pancreatic cancer cells, liver cancer cells, lung cancer cells, kidney cancer cells, sarcoma cells, breast cancer cells, brain cancer cells, neuroendocrine carcinoma cells, colon cancer cells, testicular cancer cells and melanoma cells.
52. The method of any one of subparagraphs 40-51, wherein the method further comprises constructing an image from the optical signal emitted by the compound.
53. The method of subparagraph 52, wherein the optical signal detected and/or the image constructed can be used to detect the presence or absence of the compound of in the biological sample and/or diagnose a disease.
54. The method of any one of subparagraphs 40-53, wherein the method further comprises contacting the biological tissue with a fluorescent compound, wherein the fluorescent compound has a signal property distinguishable from the signal properties of the compound of any one of subparagraphs 1-36.
55. The method of any one of subparagraphs 40-54, wherein an endoscope, catheter, tomographic system, hand-held optical imaging system, surgical goggles, or intra-operative microscope is used for the illuminating and/or detecting.
56. The method of any one of subparagraphs 42-55, wherein the compound improves the tumor-to-background ratio.
57. A method of performing image guided surgery on a subject in need thereof, said method comprising:
   administering a composition comprising a compound of any one of subparagraphs 1-36;
   illuminating the compound using an excitation light; and
   performing surgical resection of the areas that fluoresce upon excitation by the excitation light.
58. The method of subparagraph 57, wherein the method is used to treat cancer.
59. The method of subparagraph 59, wherein the cancer is selected from the group consisting of prostate cancer, lung cancer, bladder cancer, pancreatic cancer, liver cancer, kidney cancer, sarcoma, breast cancer, brain cancer, neuroendocrine carcinoma, colon cancer, testicular cancer and melanoma.
60. The method of any one of subparagraphs 57-59, wherein the compound improves surgical resection and/or provides cleaner surgical margins than non-NIR conjugated fluorescing dyes.
61. The method of any one of subparagraphs 57-60, wherein the areas that fluoresce upon excitation are tumor micro-environments or comprise tumor cells.
62. A method of diagnosing a disease in a subject in need thereof, the method comprising:
   administering a composition comprising a compound of any one of subparagraphs 1-36;
   illuminating the compound using an excitation light;
   detecting the optical signal emitted by the compound;
   comparing the signal detected with at least one control data set, wherein the at least one control data set comprises signals from the compound contacted with a biological sample that does not comprise a target cell type; and
   providing a diagnosis of disease wherein the comparison in indicates the presence of the disease.
63. The method of subparagraph 62, wherein the biological sample is selected from the group consisting of urine, blood, oral fluid, plasma, tissue, bone marrow, and tumor samples
64. The method of subparagraph 62 or subparagraph 63, wherein the disease is a metastatic disease.
65. The method of any one of subparagraphs 62-64, wherein the method improves the prognosis of the subject.
66. The method of any one of subparagraphs 41-65, wherein the subject is a mammal.
67. The method of any one of subparagraphs 41-66, wherein the subject is a human.
68. The method of any one of subparagraphs 40-67, wherein the biological sample is more than about 5 mm below the skin of the subject, alternatively more than about 6 mm below the skin of the subject, alternatively more than about 7 mm below the skin of the subject, alternatively more than about 8 mm below the skin of the subject, alternatively more than about 9 mm below the skin of the subject, or alternatively more than about 10 mm below the skin of the subject.
69. The method of any one of subparagraphs 42-56 or any one of subparagraphs 63-65, wherein the method detects tumors less than 4 mm, alternatively less than 3 mm, alternatively at least 6 mm, alternatively at least 7 mm, alternatively at least 8 mm, alternatively at least 9 mm, alternatively at least 10 mm, alternatively at least 11 mm, alternatively least 12 mm, alternatively length of at least 13 mm, alternatively at least 14 mm, alternatively at least 15 mm, alternatively at least 16 mm, alternatively at least 17 mm, alternatively at least 18 mm, alternatively least 19 mm, alternatively at least 20 mm, alternatively at least 21 mm, alternatively at least 22 mm, alternatively at least 23 mm, alternatively length of at least 24 mm, alternatively at least 25 mm, alternatively at least 30 mm.
70. The method of subparagraph 61, wherein the tumor has a length of at least 5 mm prior to surgical recession.
71. The method of any one of subparagraphs 40-70, wherein the excitation light is a near-infrared wavelength light or infrared light.
72. The method of any one of subparagraphs 40-71, wherein the excitation light has a wavelength range from about 600 nm to about 1000 nm, alternatively about 670 nm to about 850 nm, alternatively between about 650 nm to about 900 nm, alternatively between about 650 nm to about 1000 nm, alternatively about 800 nm.

The presently described technology and its advantages will be better understood by reference to the following examples. These examples are provided to describe specific implementations of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### EXAMPLES

### Example 1: Synthesis of FAPI-Targeted Conjugates

The following schemes show the synthesis of FAPI-targeted conjugates of the present invention.

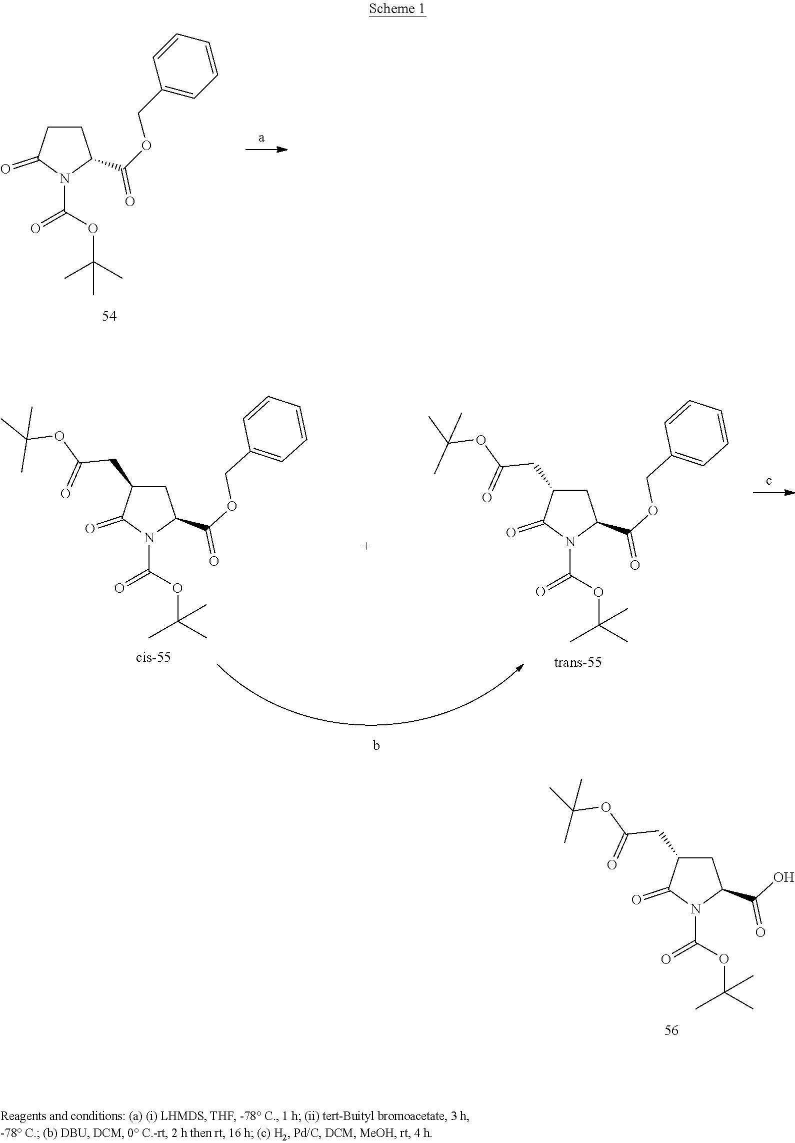 [SMILES: CC(C)(C)OC(=O)N1C(=O)CC[C@@H]1C(=O)OCc1ccccc1 ; CC(C)(C)OC(=O)C[C@H]1C[C@@H](C(=O)OCc2ccccc2)N(C(=O)OC(C)(C)C)C1=O ; CC(C)(C)OC(=O)C[C@@H]1C[C@@H](C(=O)OCc2ccccc2)N(C(=O)OC(C)(C)C)C1=O ; CC(C)(C)OC(=O)C[C@@H]1C[C@@H](C(=O)O)N(C(=O)OC(C)(C)C)C1=O]

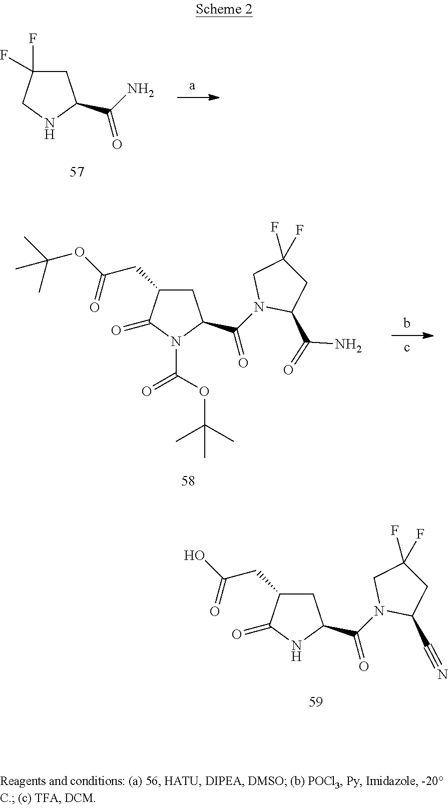 [SMILES: NC(=O)[C@@H]1CC(F)(F)CN1 ; CC(C)(C)OC(=O)C[C@@H]1C[C@@H](C(=O)N2CC(F)(F)C[C@H]2C(N)=O)N(C(=O)OC(C)(C)C)C1=O ; N#C[C@@H]1CC(F)(F)CN1C(=O)[C@@H]1C[C@@H](CC(=O)O)C(=O)N1]

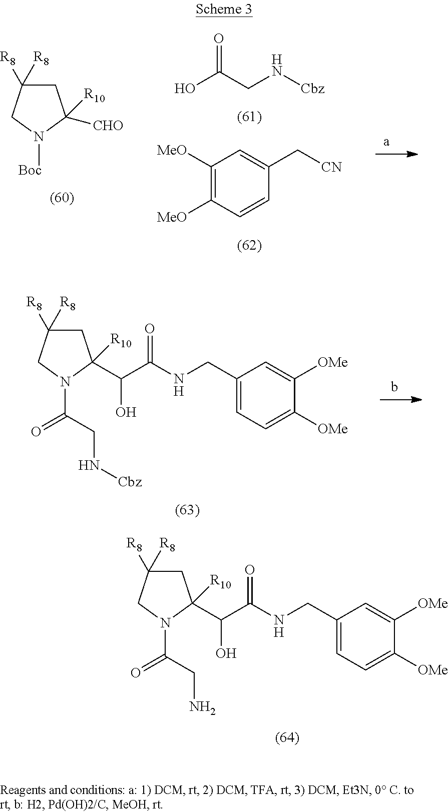

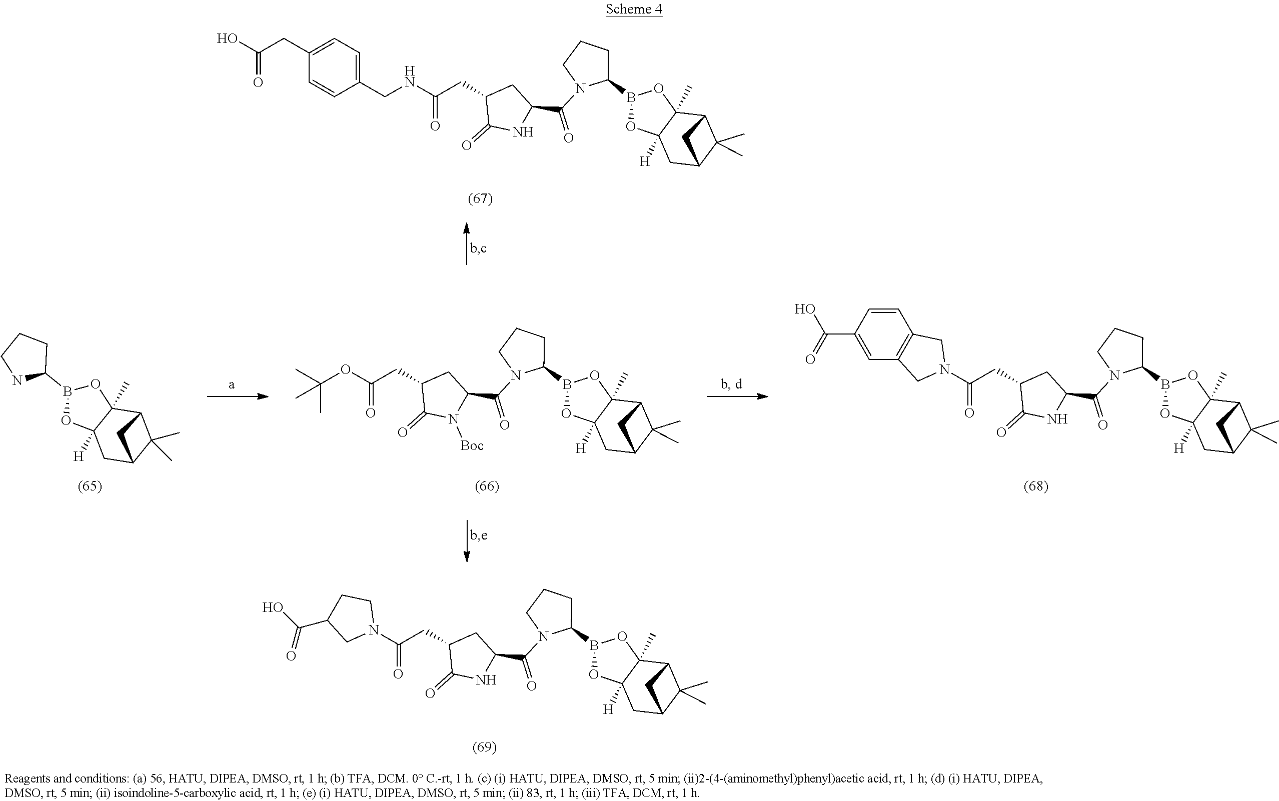

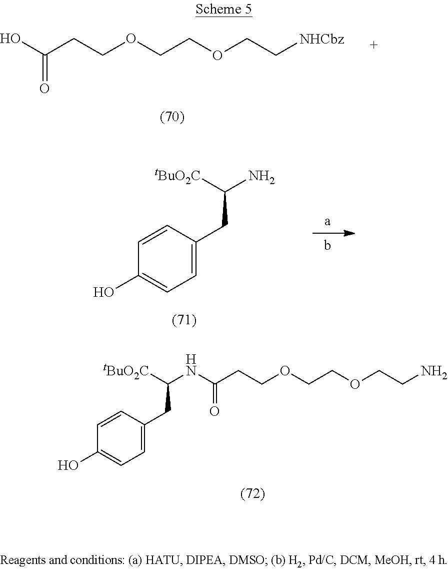 [SMILES: O=C(O)CCOCCOCCNC(=O)OCc1ccccc1 ; CC(C)(C)OC(=O)[C@@H](N)Cc1ccc(O)cc1 ; CC(C)(C)OC(=O)[C@H](Cc1ccc(O)cc1)NC(=O)CCOCCOCCN]

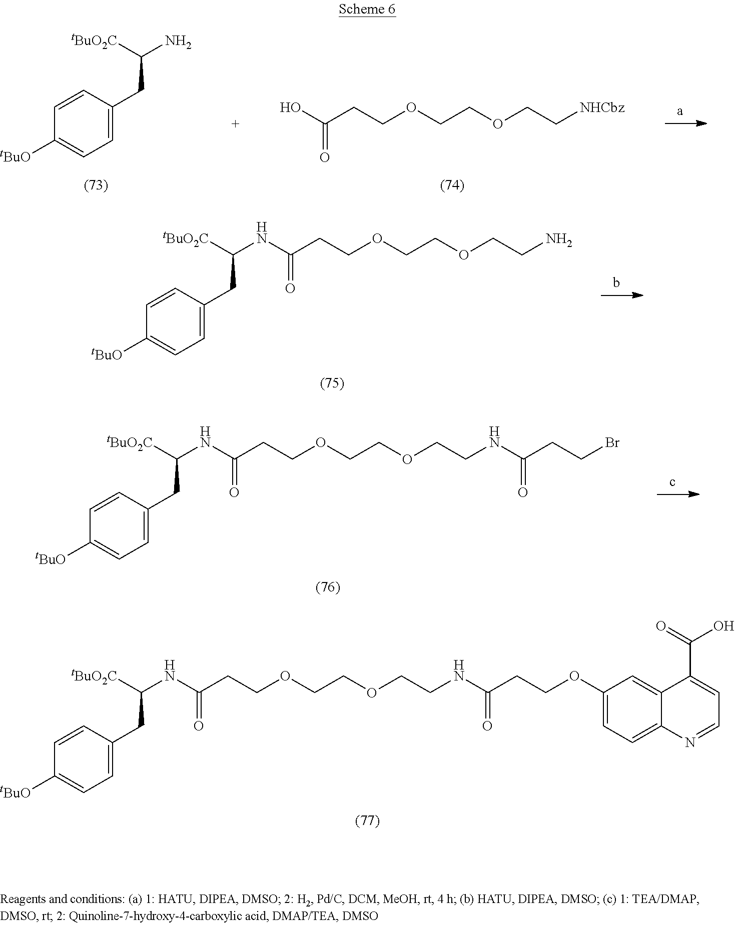 [SMILES: CC(C)(C)OC(=O)[C@@H](N)Cc1ccc(OC(C)(C)C)cc1 ; O=C(O)CCOCCOCCNC(=O)OCc1ccccc1 ; CC(C)(C)OC(=O)[C@H](Cc1ccc(OC(C)(C)C)cc1)NC(=O)CCOCCOCCN ; CC(C)(C)OC(=O)[C@H](Cc1ccc(OC(C)(C)C)cc1)NC(=O)CCOCCOCCNC(=O)CCBr ; CC(C)(C)OC(=O)[C@H](Cc1ccc(OC(C)(C)C)cc1)NC(=O)CCOCCOCCNC(=O)CCOc1ccc2nccc(C(=O)O)c2c1]

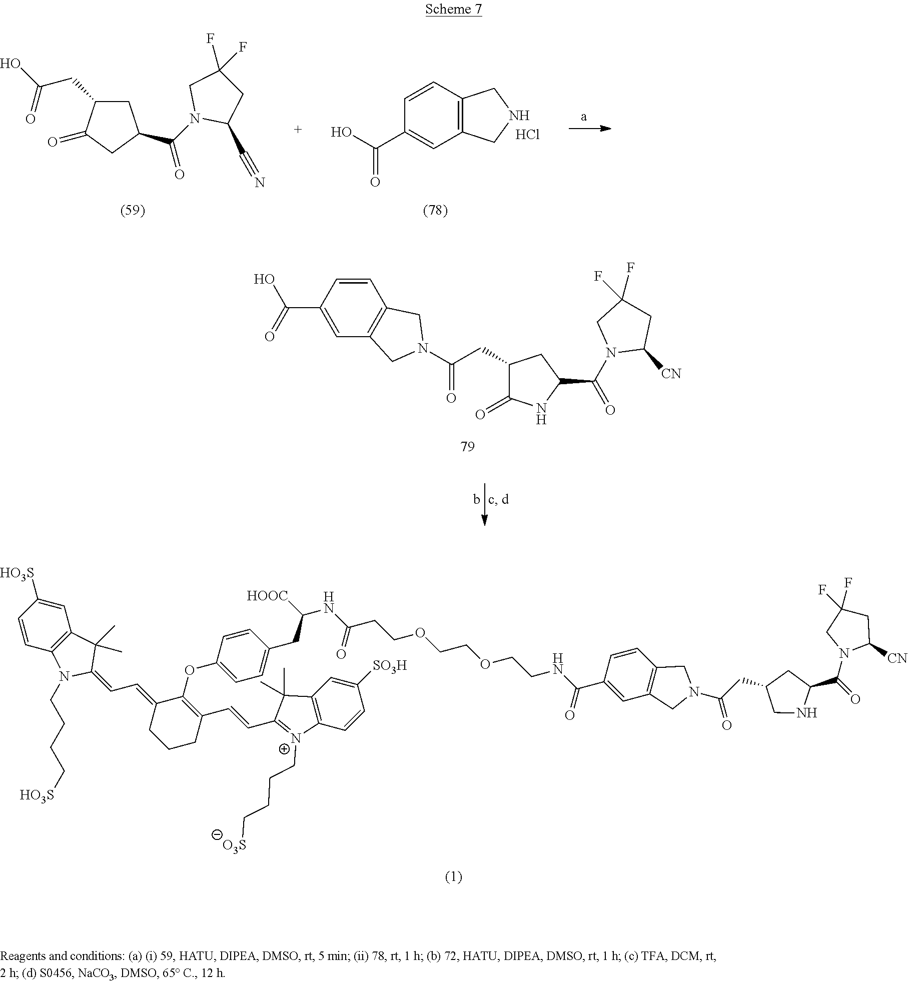

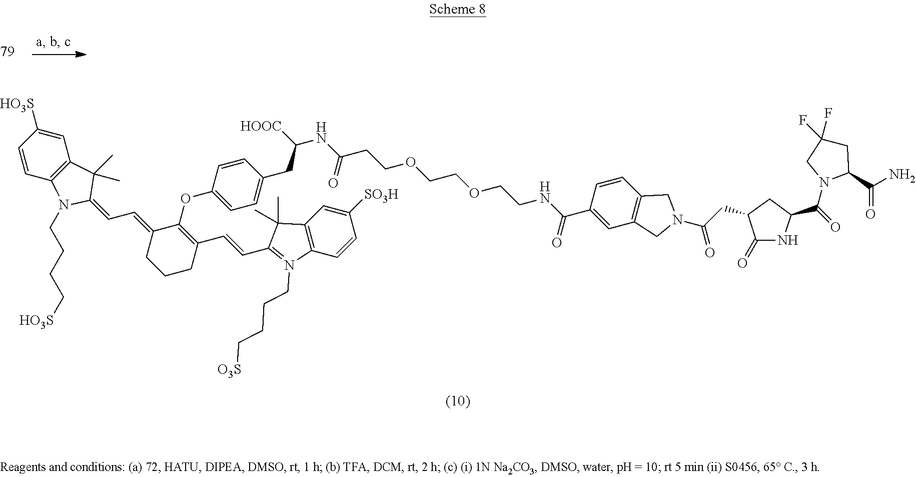

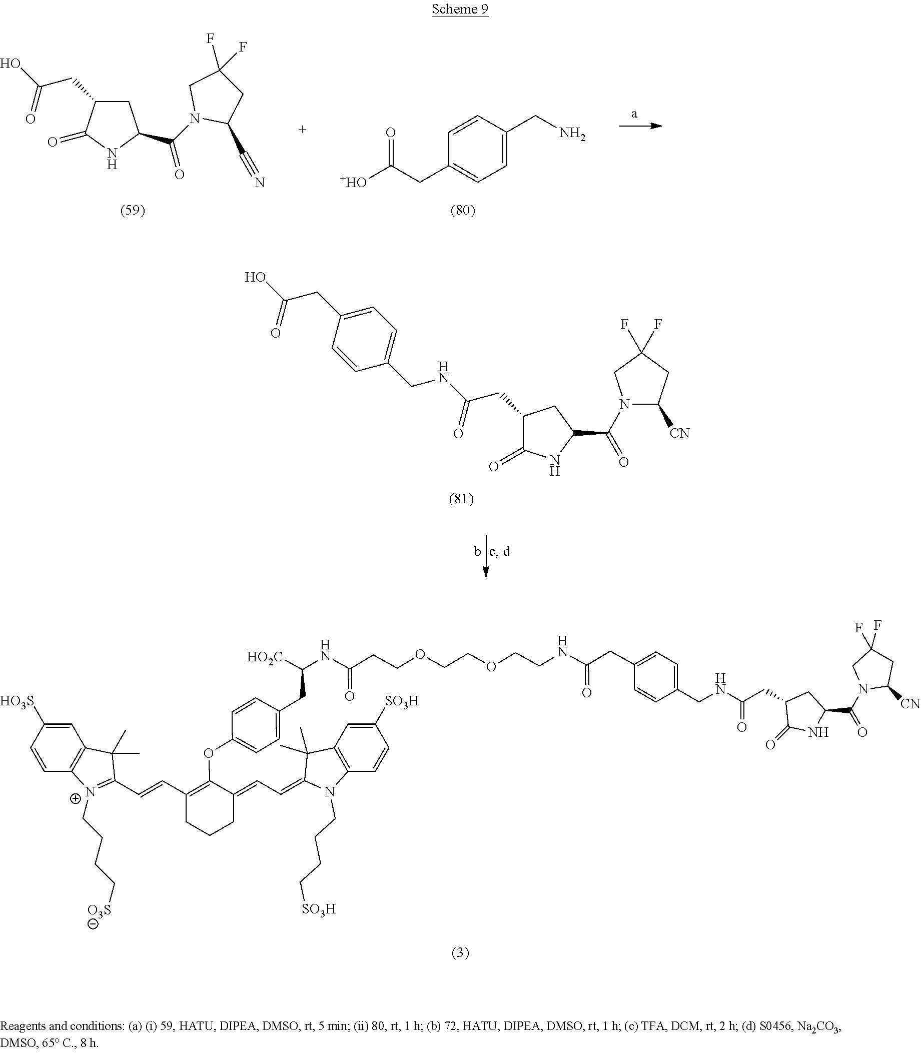

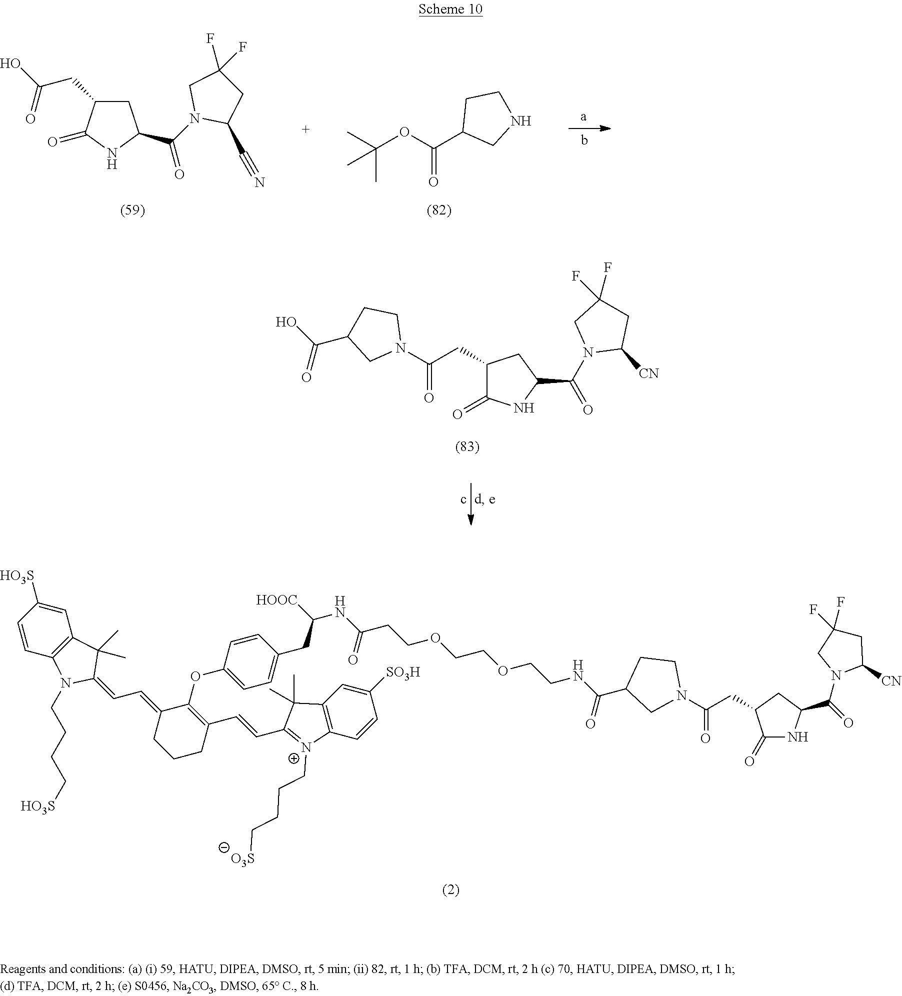

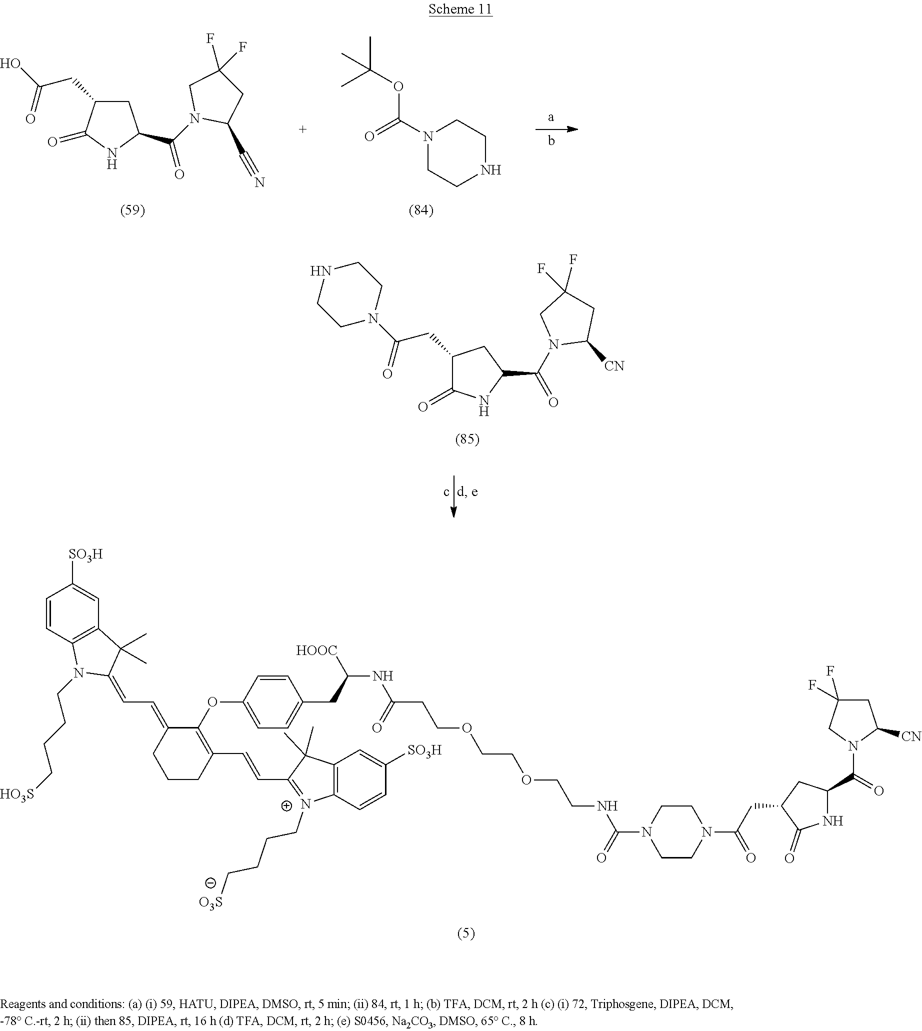

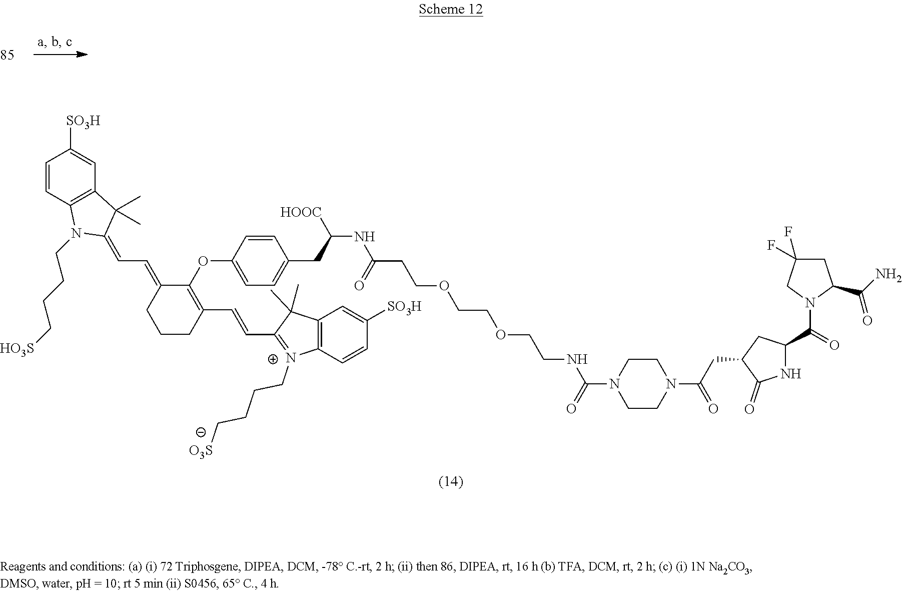

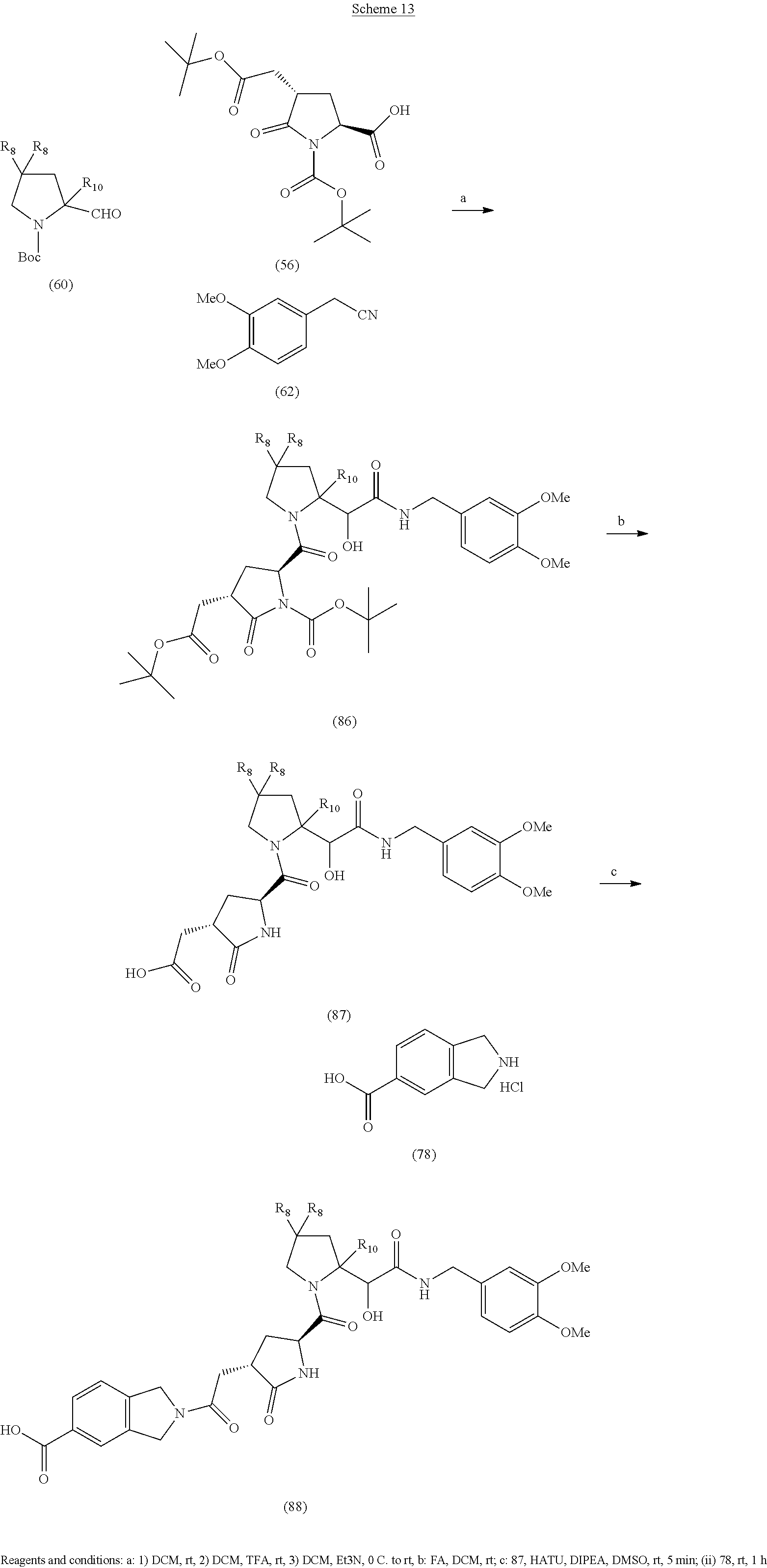

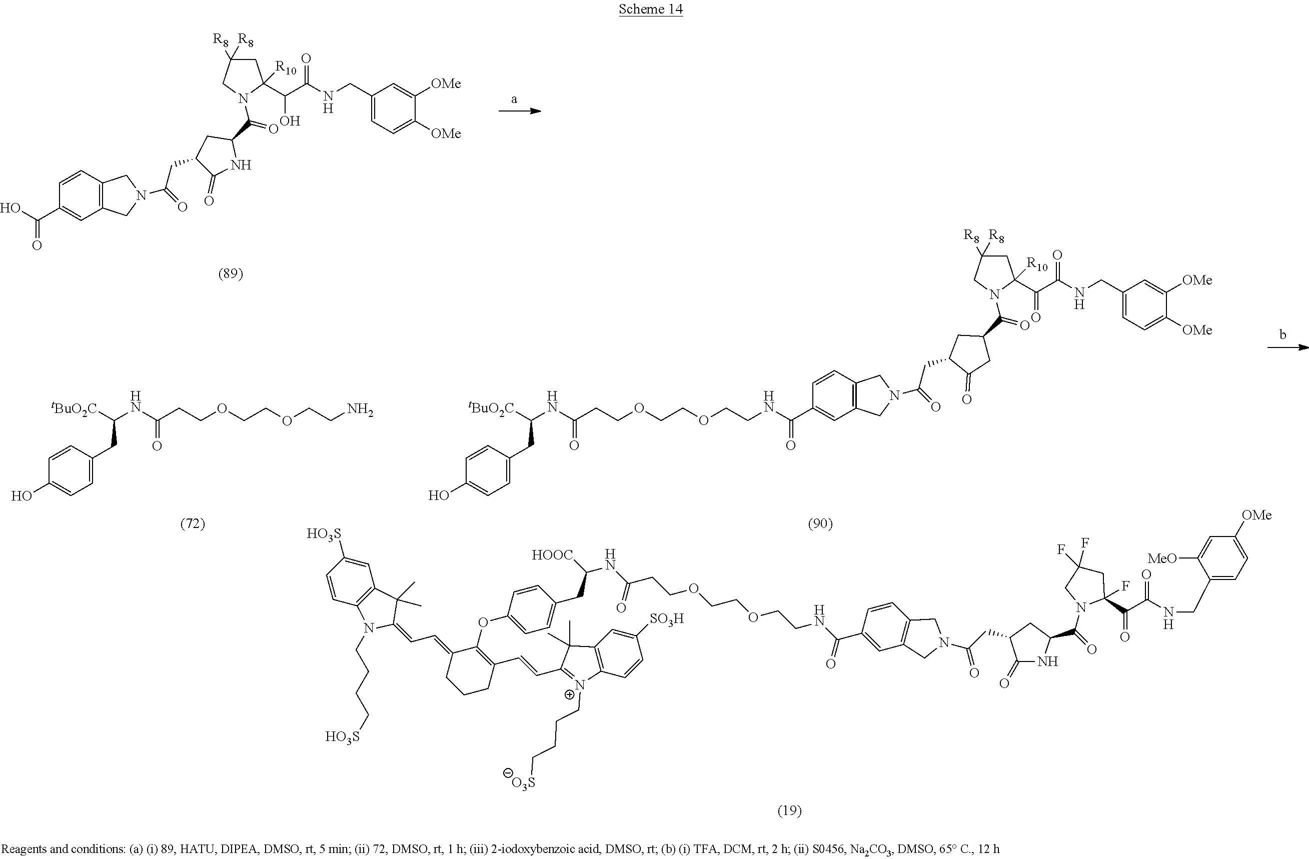

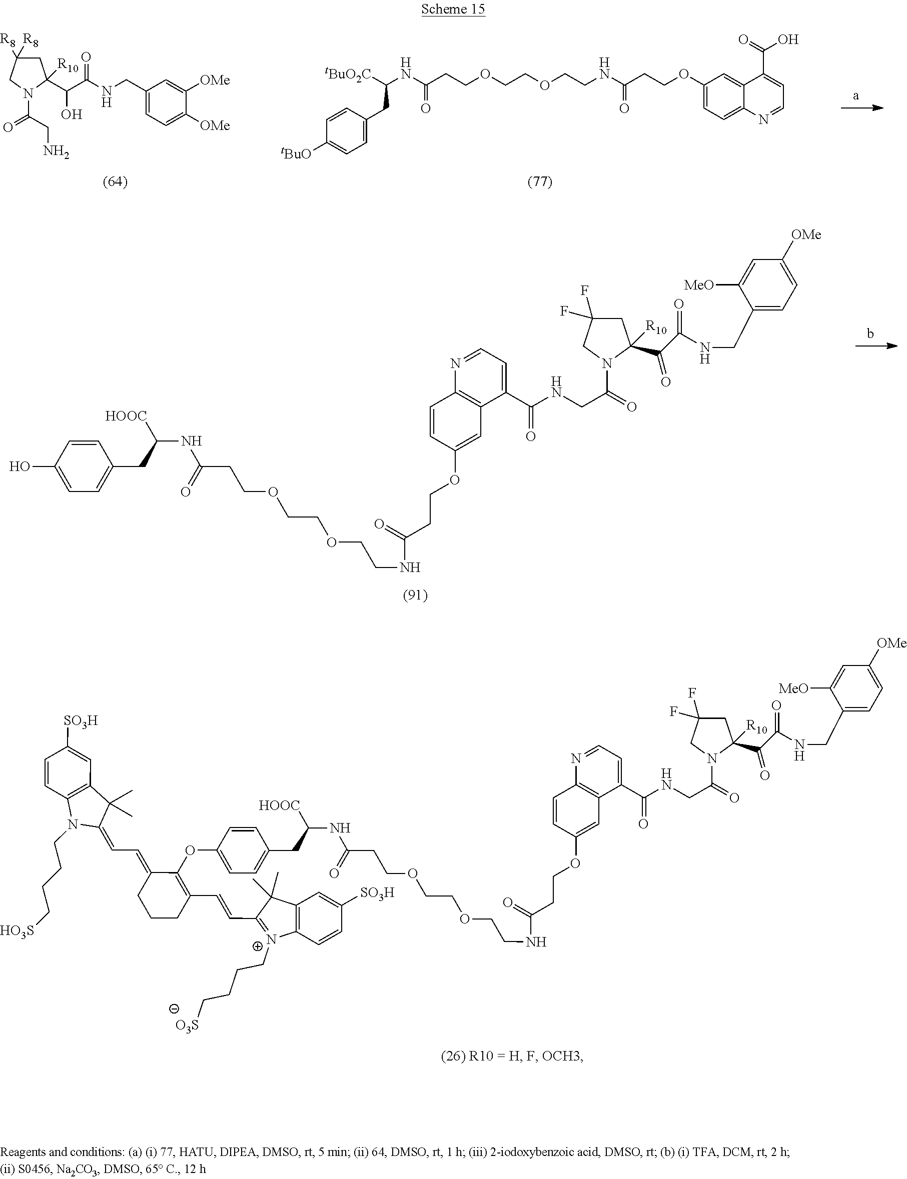

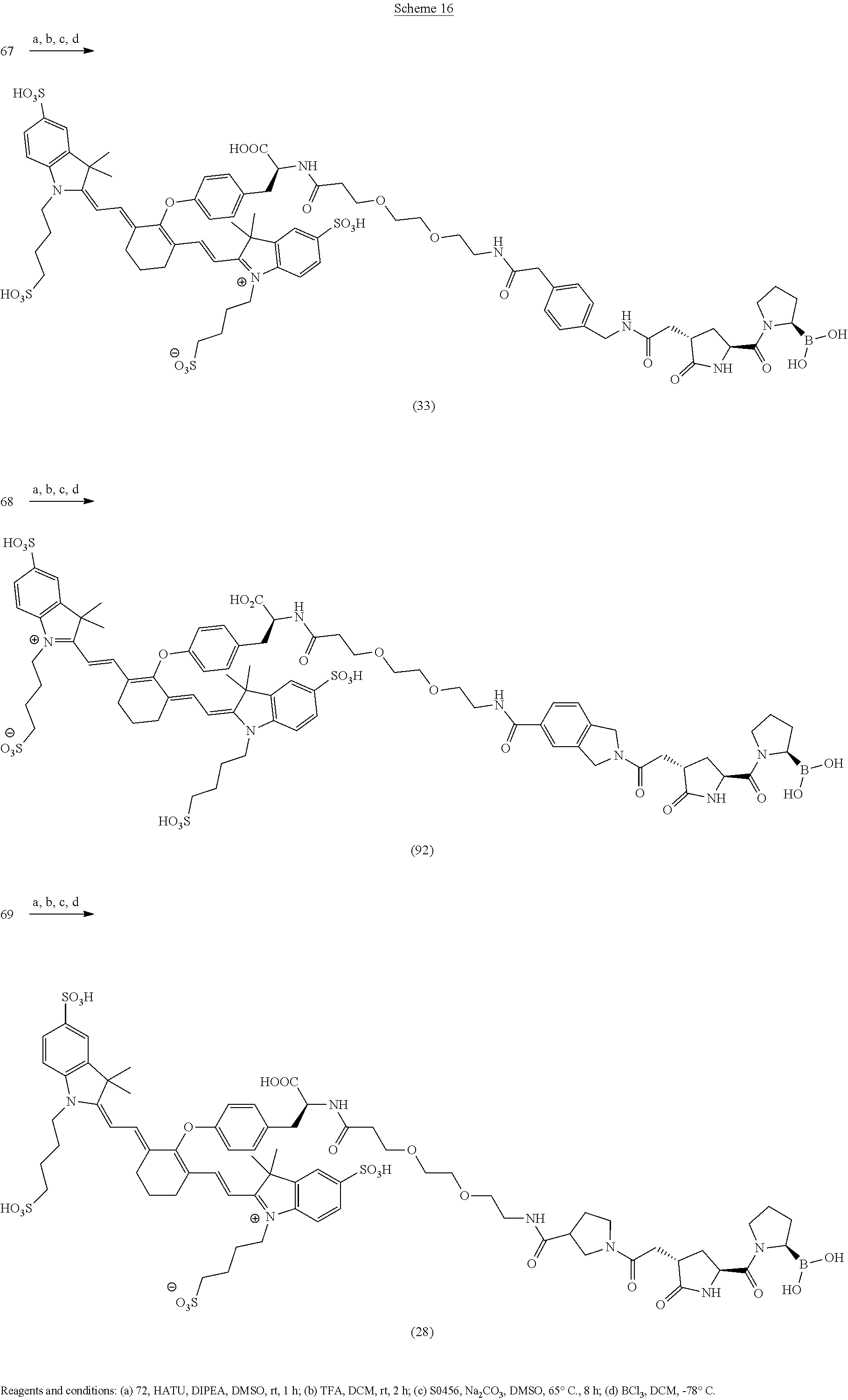

### Example 2: Chemical Synthesis Procedures of FAPI-IGS Conjugates

### 2-benzyl 1-(tert-butyl) (2S,4S)-4-(2-(tert-butoxy)-2-oxoethyl)-5-oxopyrrolidine-1,2-dicarboxylate

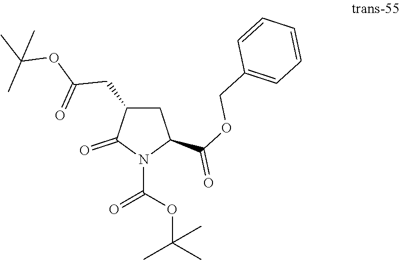 [SMILES: CC(C)(C)OC(=O)C[C@@H]1C[C@@H](C(=O)OCc2ccccc2)N(C(=O)OC(C)(C)C)C1=O]

A solution of 2-benzyl 1-(tert-butyl) (S)-5-oxopyrrolidine-1,2-dicarboxylate (5 g, 15.65 mmol) in THF (80 mL) was cooled to −20° C. in an ice-salt bath under inert atmosphere. LHMDS (1.0M, in THF, 18.78 mL, 18.78 mmol) was added dropwise over 5 min. and continued stirring for 1 h. tert-Butyl bromoacetate 2.55 mL, 17.22 mmol) was added slowly over 2 min. and stirred for 2 h at the same temperature. The reaction was monitored using LC/MS and quenched using sat. ammonium chloride aq. solution and extracted using ethyl acetate. The combined organic layer was washed with water and brine and dried (Na₂SO₄). The solvent was evaporated in vacuo to give brown oil (7.4 g). LC/MS showed the formation trans-55:cis-55 in 25:75 ratio. The crude oil of cis/trans mixture of compound 55 (7.4 g) was dissolved in the DCM (313 mL) and cooled the reaction mixture to −20° C. and DBU (6.93 mL, 46.95 mmol) was added dropwise to the reaction mixture over 20 min. and stirred while allowing it to come to rt over next 2 h and stirred at rt for 16 h. The reaction was monitored using LC/MS. The reaction mixture was washed using IN aq. HCl followed by water and brine and dried (Na₂SO₄). The solvent was evaporated in vacuo and the crude was dissolved in ether and stored at −20° C. for overnight. The crystals were filtered and washed with cold ether. This crystallization was repeated to obtain further two crops and the concentrated residue was purified by column chromatography on silica gel (Hexane:DCM:EtOAc, 4:5:1) to obtain combined cis-55 in 60% yield.

### (2S,4S)-4-(2-(tert-butoxy)-2-oxoethyl)-1-(tert-butoxycarbonyl)-5-oxopyrrolidine-2-carboxylic acid

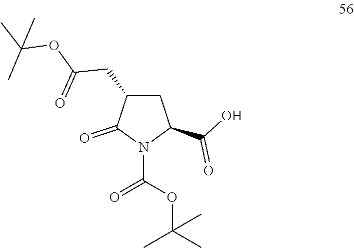 [SMILES: CC(C)(C)OC(=O)C[C@@H]1C[C@@H](C(=O)O)N(C(=O)OC(C)(C)C)C1=O]

To a solution of trans-55 (390 mg, 0.899 mmol) in dichloromethane (2 mL) stirred to dissolve and flushed the flask with nitrogen. Pd—C(80 mg, 15% by weight) was added followed by Methanol (18 mL) was added to the reaction mixture and the flask was flushed with hydrogen gas and stirred under H₂ balloon pressure. The progress of the reaction was monitored by LC/MS for 4 h. After completion of the reaction, the reaction mixture was filtered through a Celite bed and washed with DCM:methanol (1:1) mixture. The combined solvent was evaporated and crude product dried under high vacuum to obtain 652 mg of compound 56.

### tert-butyl (3S,5S)-3-(2-(tert-butoxy)-2-oxoethyl)-5-((S)-2-carbamoyl-4,4-difluoropyrrolidine-1-carbonyl)-2-oxopyrrolidine-1-carboxylate

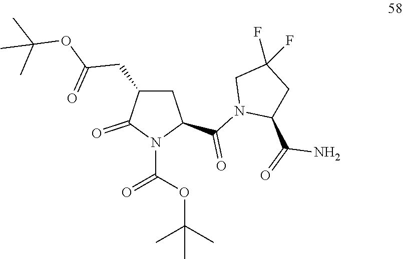 [SMILES: CC(C)(C)OC(=O)C[C@@H]1C[C@@H](C(=O)N2CC(F)(F)C[C@H]2C(N)=O)N(C(=O)OC(C)(C)C)C1=O]

A solution of compound 56 (600 mg, 1.456 mmol) and HATU (609 mg, 1.602 mmol) in DMSO (10 mL) was stirred at rt to dissolve. DIPEA (0.76 mL, 4.36 mmol) was added and stirred the content for 5 min. (S)-4,4-difluoropyrrolidine-2-carboxamide (326 mg, 1.747 mmol) was added to the above reaction mixture and stirred for 1 h. The reaction was monitored by LC/MS and quenched by adding 5% aq. Citric acid solution. Extracted using ethyl acetate and washed organics using water, brine and dried (Na₂SO₄). The organics was evaporated in vacuo and the crude was purified by column chromatography on silica gel (Hexane/EtOAc) to obtain compound 58 in 72% yield.

### 2-((3S,5S)-5-((S)-2-cyano-4,4-difluoropyrrolidine-1-carbonyl)-2-oxopyrrolidin-3-yl) acetic acid

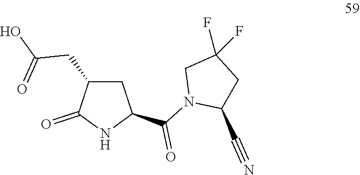 [SMILES: N#C[C@@H]1CC(F)(F)CN1C(=O)[C@@H]1C[C@@H](CC(=O)O)C(=O)N1]

A solution of 58 (822 mg, 1.73 mmol) and imidazole (129 mg, 1.90 mmol) in pyridine (8.5 mL) was cooled to −20° C. in an ice-salt bath under inert atmosphere. POCl₃ (0.404 mL, 4.32 mmol) was added dropwise over 5 min. and continued stirring at the same temperature for 30 min. The reaction was monitored by LC/MS and evaporated solvent in vacuo and diluted the residue using DCM. Washed the organics with 5% aq. Citric acid, water, brine and dried (Na₂SO₄). The solvent was evaporated in vacuo and the crude was purified by column chromatography on silica gel (Hexane/EtOAc) to obtain tert-butyl (3S,5S)-3-(2-(tert-butoxy)-2-oxoethyl)-5-((S)-2-cyano-4,4-difluoropyrrolidine-1-carbonyl)-2-oxopyrrolidine-1-carboxylate (632 mg) in 80% yield. Which was dissolved in DCM (2 mL) and TFA (5 mL) was added to it slowly and stirred the content for 1 h. The reaction was monitored by LC/MS and solvent was evaporated in vacuo and to the residue ether was added. The precipitated solid was filtered and washed with ether and dried under vacuum to obtain compound 59 in 90% yield.

### tert-butyl (3-(2-(2-aminoethoxy) ethoxy) propanoyl)-L-tyrosinate

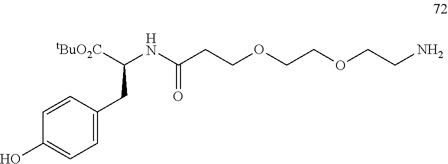 [SMILES: CC(C)(C)OC(=O)[C@H](Cc1ccc(O)cc1)NC(=O)CCOCCOCCN]

To a solution of Cbz-N-amido-PEG2-acid (5 g, 16.06 mmol) in DMSO (53.5 mL) and DIPEA (83.9 mL, 4.81 mmol) was added under argon atmosphere. The reaction mixture was stirred at room temperature for 30 min (until clear solution is formed). HATU (6.290 g, 16.542 mmol) was added to the reaction mixture portion wise using addition funnel over 15 min. The reaction mixture turned to pale yellow to yellowish brown color. The reaction mixture was stirred at room temperature for additional 15 min. tert-butyl L-tyrosinate (3.49 g, 16.221 mmol) was added to the reaction mixture over 5 min and stirred for 15 min at 23° C. The reaction progress was monitored by LC/MS. The reaction mixture was added to a stirred solution of IN HCl (500 mL) over 10 min and stirred for 15 min and aqueous layer was extracted with ethyl acetate. The combined organic layers were washed with water, brine and dried over sodium sulfate. The organics was evaporated in vacuo and the crude was purified by column chromatography on silica gel (Hexane/EtOAc) to obtain compound tert-butyl (3-(2-(2-aminoethoxy) ethoxy) propanoyl)-L-tyrosinate in 73% yield. This compound was then dissolved in DCM (60 mL) under argon and was stirred at room temperature for 20 min (until clear solution is formed). After flushing the flask with argon (2×), Pd/C (920 mg, 15% by weight) was added to the reaction mixture and methanol (60 mL) was added to the reaction mixture and the flask was flushed with hydrogen gas and stirred under H₂ balloon pressure for 8 h. The progress of the reaction was monitored by LC/MS. After completion of the reaction, the reaction mixture was filtered through a Celite bed and washed with dichloromethane:methanol (1:1) mixture. The combined solvent was evaporated and crude product dried under high vacuum to obtain 4.4 g of desired product 72 in 95.8% yield.

### 2-(2-((3S,5R)-5-((S)-2-cyano-4,4-difluoropyrrolidine-1-carbonyl)-2-oxopyrrolidin-3-yl) acetyl) isoindoline-5-carboxylic acid

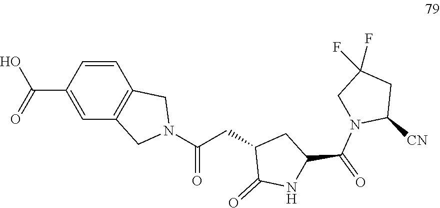

To a solution of 59 (80 mg, 0.265 mmol) in DMSO (5 mL) and DIPEA (0.2 mL, 2.12 mmol) was added under argon atmosphere. The reaction mixture was stirred at room temperature for 5 min (until clear solution is formed). HATU (101 mg, 0.165 mmol) was added to the reaction mixture. The reaction mixture turned to pale yellow to yellowish brown color. The reaction mixture was stirred at room temperature for additional 15 min. Isoindoline-5-carboxylic acid hydrochloride (53 mg, 0.165 mmol) was added to the reaction mixture for 1 h. The reaction progress was monitored by LC/MS. The reaction mixture was added to a stirred solution of 5% aq. Citric acid and aqueous layer was extracted with ethyl acetate. The combined organic layer was washed with water, brine and dried over sodium sulfate. The solvent was evaporated in vacuo and the crude was purified by column chromatography on silica gel (Hexane/EtOAc) to obtain 79 in 83% yield.

### (3-(2-(2-(2-(2-((3S,5S)-5-((S)-2-cyano-4,4-difluoropyrrolidine-1-carbonyl)-2-oxopyrrolidin-3-yl) acetyl) isoindoline-5-carboxamido)ethoxy)ethoxy)propanoyl)-L-tyrosine

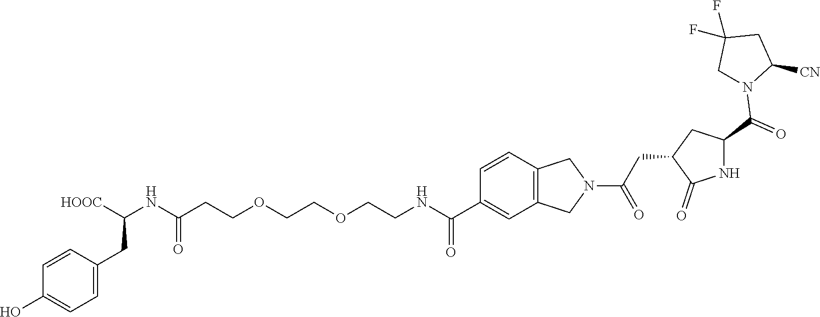 [SMILES: N#C[C@@H]1CC(F)(F)CN1C(=O)[C@@H]1C[C@@H](CC(=O)N2Cc3ccc(C(=O)NCCOCCOCCC(=O)N[C@@H](Cc4ccc(O)cc4)C(=O)O)cc3C2)C(=O)N1]

To a solution of 79 (120 mg, 0.268 mmol) and 72 (106 mg, 0.268 mmol) in DMSO (5 mL) and DIPEA (140 mL, 0.806 mmol) was added under argon atmosphere. The reaction mixture was stirred at room temperature for 5 min (until clear solution is formed). PyBop (168 mg, 0.322 mmol) was added to the reaction mixture. The reaction mixture turned to pale yellow to yellowish brown color. The reaction progress was monitored by LC/MS. The reaction mixture was added to a stirred solution of 5% aq. Citric acid and aqueous layer was extracted with ethyl acetate. The combined organic layer was washed with water, brine and dried over sodium sulfate. The solvent was evaporated in vacuo and the crude was purified by column chromatography on silica gel (DCM/MeOH) to obtain tert-butyl (3-(2-(2-(2-(2-((3S,5S)-5-((S)-2-cyano-4,4-difluoropyrrolidine-1-carbonyl)-2-oxopyrrolidin-3-yl) acetyl) isoindoline-5-carboxamido)ethoxy)ethoxy)propanoyl)-L-tyrosinate in 187 mg (85% yield), which is dissolved in DCM (2 mL) and TFA (2 mL) was added slowly and stirred the content at rt for 2 h. The reaction was monitored by LC/MS and solvent was evaporated in vacuo and to the residue ether was added. The precipitated solid was filtered and washed with ether and dried under vacuum to obtain 3-(2-(2-(2-(2-((3S,5S)-5-((S)-2-cyano-4,4-difluoropyrrolidine-1-carbonyl)-2-oxopyrrolidin-3-yl) acetyl) isoindoline-5-carboxamido)ethoxy)ethoxy)propanoyl)-L-tyrosine in 90% yield.

### 4-(2-((E)-2-((E)-2-(4-((S)-13-carboxy-1-(2-(2-((3S,5S)-5-((S)-2-cyano-4,4-difluoropyrrolidine-1-carbonyl)-2-oxopyrrolidin-3-yl) acetyl) isoindolin-5-yl)-1,11-dioxo-5,8-dioxa-2,12-diazatetradecan-14-yl) phenoxy)-3-(2-((E)-3,3-dimethyl-5-sulfo-1-(4-sulfobutyl) indolin-2-ylidene)ethylidene) cyclohex-1-en-1-yl) vinyl)-3,3-dimethyl-5-sulfo-3H-indol-1-ium-1-yl) butane-1-sulfonate

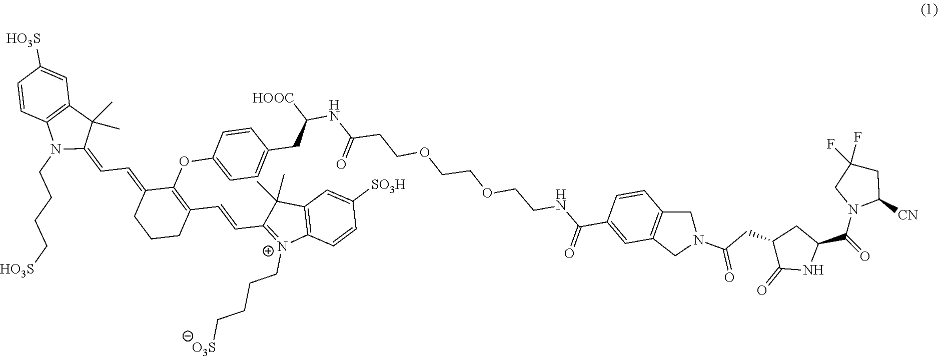

A 5 mL round bottom flask was charged with 3-(2-(2-(2-(2-((3S,5S)-5-((S)-2-cyano-4,4-difluoropyrrolidine-1-carbonyl)-2-oxopyrrolidin-3-yl) acetyl) isoindoline-5-carboxamido)ethoxy)ethoxy)propanoyl)-L-tyrosine (5 mg, 0.0065 mmol), Na₂CO₃ (6.9 mg, 0.065 mmol) and S0456 (6.5 mg, 0.068 mmol) and DMSO (0.6 mL) was added and stirred the content at 65° C. oil bath for 6 h. The reaction was monitored by LC/MS and formation of 1 along with amide-2 was also observed. The reaction mixture was cooled to rt and purified by RP-HPLC to obtain the compound 1 in 23% and 10 in 20% yield.

### 4-(2-((E)-2-((E)-2-(4-((S)-1-(2-(2-((3S,5S)-5-((S)-2-carbamoyl-4,4-difluoropyrrolidine-1-carbonyl)-2-oxopyrrolidin-3-yl) acetyl) isoindolin-5-yl)-13-carboxy-1,11-dioxo-5,8-dioxa-2,12-diazatetradecan-14-yl) phenoxy)-3-(2-((E)-3,3-dimethyl-5-sulfo-1-(4-sulfobutyl) indolin-2-ylidene)ethylidene) cyclohex-1-en-1-yl) vinyl)-3,3-dimethyl-5-sulfo-3H-indol-1-ium-1-yl) butane-1-sulfonate

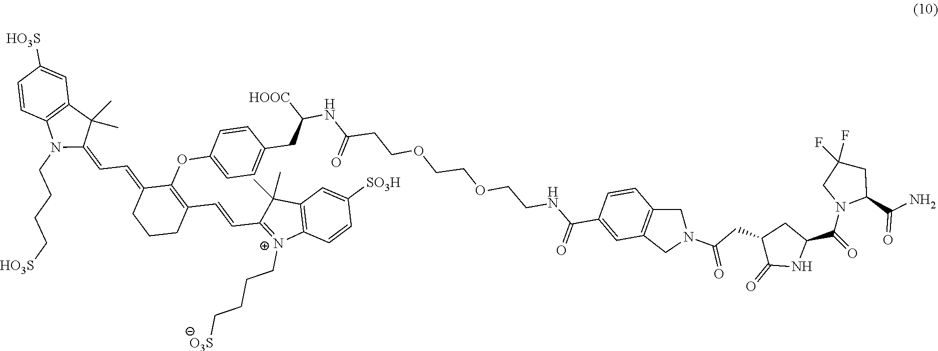

### 2-(4-((2-((3S,5S)-5-((S)-2-cyano-4,4-difluoropyrrolidine-1-carbonyl)-2-oxopyrrolidin-3-yl) acetamido)methyl)phenyl) acetic acid

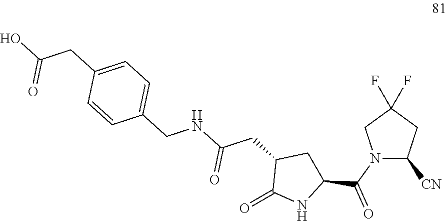 [SMILES: N#C[C@@H]1CC(F)(F)CN1C(=O)[C@@H]1C[C@@H](CC(=O)NCc2ccc(CC(=O)O)cc2)C(=O)N1]

To a solution of 59 (80 mg, 0.265 mmol) in DMSO (5 mL) and DIPEA 0.138 mL, 0.796 mmol) was added under argon atmosphere. The reaction mixture was stirred at room temperature for 5 min (until clear solution is formed). HATU (106 mg, 0.278 mmol) was added to the reaction mixture. The reaction mixture turned to pale yellow to yellowish brown color. The reaction mixture was stirred at room temperature for additional 15 min and 2-(4-(aminomethyl)phenyl) acetic acid (44 mg, 0.265 mmol) was added to the reaction mixture for 1 h. The reaction progress was monitored by LC/MS. The reaction mixture was added to a stirred solution of 5% aq. Citric acid and aqueous layer was extracted with ethyl acetate. The combined organic layer was washed with water, brine and dried over sodium sulfate. The solvent was evaporated in vacuo and the crude was purified by column chromatography on silica gel (MeOH/EtOAc) to obtain 81 in 84% yield.

### (3-(2-(2-(2-(4-((2-((3S,5S)-5-((S)-2-cyano-4,4-difluoropyrrolidine-1-carbonyl)-2-oxopyrrolidin-3-yl) acetamido)methyl)phenyl) acetamido) ethoxy) ethoxy) propanoyl)-L-tyrosine

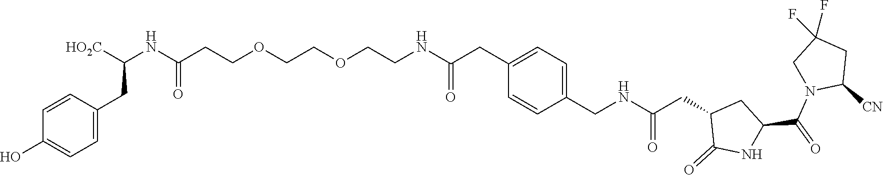 [SMILES: N#C[C@@H]1CC(F)(F)CN1C(=O)[C@@H]1C[C@@H](CC(=O)NCc2ccc(CC(=O)NCCOCCOCCC(=O)N[C@@H](Cc3ccc(O)cc3)C(=O)O)cc2)C(=O)N1]

To a solution of 81 (50 mg, 0.11 mmol) and 72 (44 mg, 0.11 mmol) in DMSO (2 mL) and DIPEA (0.194 mL, 1.12 mmol) was added under argon atmosphere. The reaction mixture was stirred at room temperature for 5 min (until clear solution is formed). PyBop (58 mg, 0.11 mmol) was added to the reaction mixture. The reaction mixture turned to pale yellow to yellowish brown color. The reaction progress was monitored by LC/MS. The reaction mixture was added to a stirred solution of 5% aq. Citric acid and aqueous layer was extracted with ethyl acetate. The combined organic layer was washed with water, brine and dried over sodium sulfate. The solvent was evaporated in vacuo and the crude was purified by column chromatography on silica gel (DCM/MeOH) to obtain tert-butyl (3-(2-(2-(2-(4-((2-((3S,5S)-5-((S)-2-cyano-4,4-difluoropyrrolidine-1-carbonyl)-2-oxopyrrolidin-3-yl) acetamido)methyl)phenyl) acetamido) ethoxy) ethoxy) propanoyl)-L-tyrosinate, 70 mg (77% yield), which is dissolved in DCM (2 mL) and TFA (2 mL) was added slowly and stirred the content at rt for 2 h. The reaction was monitored by LC/MS and solvent was evaporated in vacuo and to the residue ether was added. The precipitated solid was filtered and washed with ether and dried under vacuum to obtain (3-(2-(2-(2-(4-((2-((3S,5S)-5-((S)-2-cyano-4,4-difluoropyrrolidine-1-carbonyl)-2-oxopyrrolidin-3-yl) acetamido)methyl)phenyl) acetamido) ethoxy) ethoxy) propanoyl)-L-tyrosine in 95% yield.

### 4-(2-((E)-2-((E)-2-(4-((S)-14-carboxy-1-(4-((2-((3S,5S)-5-((S)-2-cyano-4,4-difluoropyrrolidine-1-carbonyl)-2-oxopyrrolidin-3-yl) acetamido)methyl)phenyl)-2,12-dioxo-6,9-dioxa-3,13-diazapentadecan-15-yl) phenoxy)-3-(2-((E)-3,3-dimethyl-5-sulfo-1-(4-sulfobutyl) indolin-2-ylidene)ethylidene) cyclohex-1-en-1-yl) vinyl)-3,3-dimethyl-5-sulfo-3H-indol-1-ium-1-yl) butane-1-sulfonate

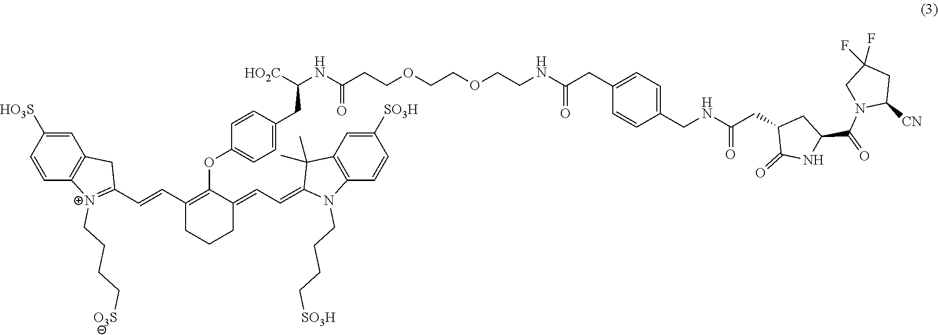

A 5 mL round bottom flask was charged with (3-(2-(2-(2-(4-((2-((3S,5S)-5-((S)-2-cyano-4,4-difluoropyrrolidine-1-carbonyl)-2-oxopyrrolidin-3-yl) acetamido)methyl)phenyl) acetamido) ethoxy) ethoxy) propanoyl)-L-tyrosine (15 mg, 0.019 mmol), Na₂CO₃ (23.7 mg, 0.19 mmol) and S0456 (22 mg, 0.023 mmol) and DMSO (0.6 mL) was added and stirred the content at 65° C. oil bath for 6 h. The reaction was monitored by LC/MS, the reaction mixture was cooled to rt and purified by RP-HPLC to obtain the compound 3 in 42% yield.

### 1-(2-((3S,5S)-5-((S)-2-cyano-4,4-difluoropyrrolidine-1-carbonyl)-2-oxopyrrolidin-3-yl) acetyl) pyrrolidine-3-carboxylic acid

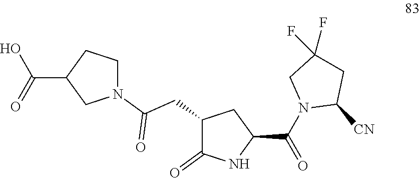 [SMILES: N#C[C@@H]1CC(F)(F)CN1C(=O)[C@@H]1C[C@@H](CC(=O)N2CCC(C(=O)O)C2)C(=O)N1]

To a solution of 59 (80 mg, 0.265 mmol) in DMSO (5 mL) and DIPEA 0.138 mL, 0.796 mmol) was added under argon atmosphere. The reaction mixture was stirred at room temperature for 5 min (until clear solution is formed). HATU (106 mg, 0.278 mmol) was added to the reaction mixture. The reaction mixture turned to pale yellow to yellowish brown color. The reaction mixture was stirred at room temperature for additional 15 min tert-butyl pyrrolidine-3-carboxylate (45 mg, 0.265 mmol) was added to the reaction mixture for 1 h. The reaction progress was monitored by LC/MS. The reaction mixture was added to a stirred solution of 5% aq. Citric acid and aqueous layer was extracted with ethyl acetate. The combined organic layer was washed with water, brine and dried over sodium sulfate. The solvent was evaporated in vacuo and the crude was purified by column chromatography on silica gel (MeOH/EtOAc) to obtain tert-butyl 1-(2-((3S,5S)-5-((S)-2-cyano-4,4-difluoropyrrolidine-1-carbonyl)-2-oxopyrrolidin-3-yl) acetyl) pyrrolidine-3-carboxylate in 81% yield, which was dissolved in DCM (2 mL) and TFA (2 mL) was added slowly and stirred the content at rt for 1 h. The reaction was monitored by LC/MS and solvent was evaporated in vacuo and to the residue ether was added. The precipitated solid was filtered and washed with ether and dried under vacuum to obtain 83 in 76% yield.

(3-(2-(2-(1-(2-((3S,5S)-5-((S)-2-cyano-4,4-difluoropyrrolidine-1-carbonyl)-2-oxopyrrolidin-3-yl) acetyl) pyrrolidine-3-carboxamido)ethoxy)ethoxy)propanoyl)-L-tyrosine

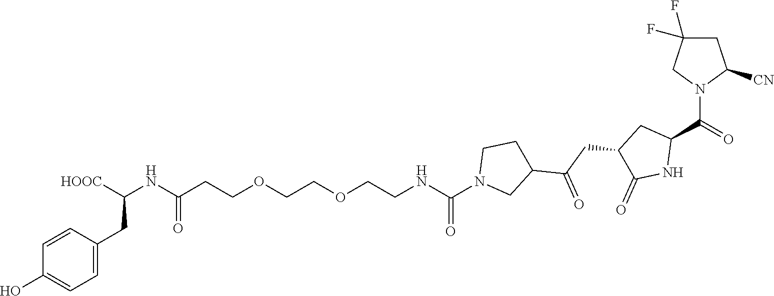 [SMILES: N#C[C@@H]1CC(F)(F)CN1C(=O)[C@@H]1C[C@@H](CC(=O)C2CCN(C(=O)NCCOCCOCCC(=O)N[C@@H](Cc3ccc(O)cc3)C(=O)O)C2)C(=O)N1]

To a solution of 83 (10 mg, 0.025 mmol) and 72 (11 mg, 0.027 mmol) in DMSO (0.6 mL) and DIPEA (0.018 mL, 0.10 mmol) was added under argon atmosphere. The reaction mixture was stirred at room temperature for 5 min (until clear solution is formed). HATU (10 mg, 0.026 mmol) was added to the reaction mixture. The reaction mixture turned to pale yellow to yellowish brown color. The reaction progress was monitored by LC/MS. The reaction mixture was added to a stirred solution of 5% aq. Citric acid and aqueous layer was extracted with ethyl acetate. The combined organic layer was washed with water, brine and dried over sodium sulfate. The solvent was evaporated in vacuo and the crude was purified by column chromatography on silica gel (DCM/MeOH) to obtain, tert-butyl (3-(2-(2-(1-(2-((3S,5S)-5-((S)-2-cyano-4,4-difluoropyrrolidine-1-carbonyl)-2-oxopyrrolidin-3-yl) acetyl) pyrrolidine-3-carboxamido)ethoxy)ethoxy)propanoyl)-L-tyrosinate 10 mg, which is dissolved in DCM (1 mL) and TFA (1 mL) was added slowly and stirred the content at rt for 2 h. The reaction was monitored by LC/MS and solvent was evaporated in vacuo and to the residue ether was added. The precipitated solid was filtered and washed with ether and dried under vacuum to obtain (3-(2-(2-(1-(2-((3S,5S)-5-((S)-2-cyano-4,4-difluoropyrrolidine-1-carbonyl)-2-oxopyrrolidin-3-yl) acetyl) pyrrolidine-3-carboxamido)ethoxy)ethoxy)propanoyl)-L-tyrosine in 88% yield.

### 4-(2-((E)-2-((E)-2-(4-((13S)-13-carboxy-1-(1-(2-((3S,5S)-5-((S)-2-cyano-4,4-difluoropyrrolidine-1-carbonyl)-2-oxopyrrolidin-3-yl) acetyl) pyrrolidin-3-yl)-1,11-dioxo-5,8-dioxa-2,12-diazatetradecan-14-yl) phenoxy)-3-(2-((E)-3,3-dimethyl-5-sulfo-1-(4-sulfobutyl) indolin-2-ylidene)ethylidene) cyclohex-1-en-1-yl) vinyl)-3,3-dimethyl-5-sulfo-3H-indol-1-ium-1-yl) butane-1-sulfonate

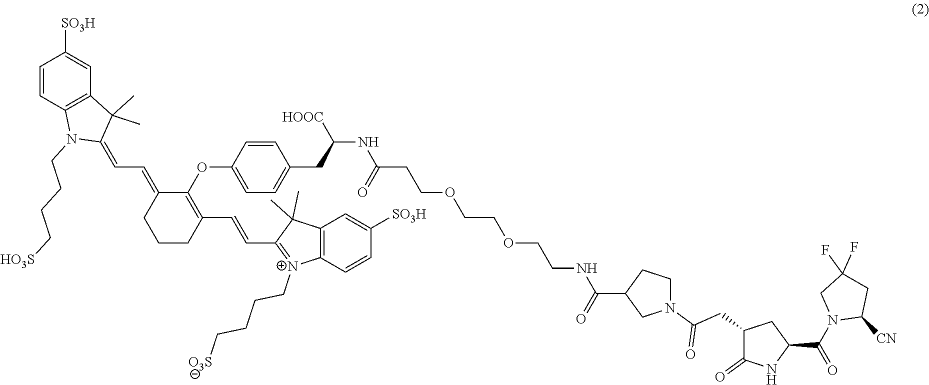

A 5 mL round bottom flask was charged with (3-(2-(2-(1-(2-((3S,5S)-5-((S)-2-cyano-4,4-difluoropyrrolidine-1-carbonyl)-2-oxopyrrolidin-3-yl) acetyl) pyrrolidine-3-carboxamido) ethoxy) ethoxy) propanoyl)-L-tyrosine (8 mg, 0.011 mmol), Na₂CO₃ (11.8 mg, 0.11 mmol) and S0456 (10.6 mg, 0.011 mmol) and DMSO (0.8 mL) was added and stirred the content at 65° C. oil bath for 9 h. The reaction was monitored by LC/MS, the reaction mixture was cooled to rt and purified by RP-HPLC to obtain the compound 2 in 25% yield.

### (S)-4,4-difluoro-1-((2S,4S)-5-oxo-4-(2-oxo-2-(piperazin-1-yl)ethyl) pyrrolidine-2-carbonyl) pyrrolidine-2-carbonitrile

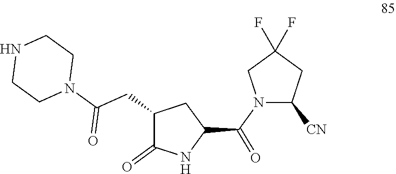

To a solution of 59 (60 mg, 0.199 mmol) in DMSO (4 mL) and

DIPEA 0.173 mL, 0.995 mmol) was added under argon atmosphere. The reaction mixture was stirred at room temperature for 5 min (until clear solution is formed). HATU (83 mg, 0.219 mmol) was added to the reaction mixture. The reaction mixture turned to pale yellow to yellowish brown color. The reaction mixture was stirred at room temperature for additional 15 min and 84 (41 mg, 0.219 mmol) was added to the reaction mixture for 1 h. The reaction progress was monitored by LC/MS. The reaction mixture was added to a stirred solution of 5% aq. Citric acid and aqueous layer was extracted with ethyl acetate. The combined organic layer was washed with water, brine and dried over sodium sulfate. The solvent was evaporated in vacuo and the crude was purified by column chromatography on silica gel (MeOH/DCM) to obtain tert-butyl 4-(2-((3S,5S)-5-((S)-2-cyano-4,4-difluoropyrrolidine-1-carbonyl)-2-oxopyrrolidin-3-yl)acetyl)piperazine-1-carboxylate in 60 mg, which was dissolved in DCM (1 mL) and TFA (1 mL) was added slowly and stirred the content at rt for 1 h. The reaction was monitored by LC/MS and solvent was evaporated in vacuo and to the residue ether was added. The precipitated solid was filtered and washed with ether and dried under vacuum to obtain 85 in 98% yield.

### (3-(2-(2-(4-(2-((3S,5S)-5-((S)-2-cyano-4,4-difluoropyrrolidine-1-carbonyl)-2-oxopyrrolidin-3-yl)acetyl)piperazine-1-carboxamido)ethoxy)ethoxy)propanoyl)-L-tyrosine

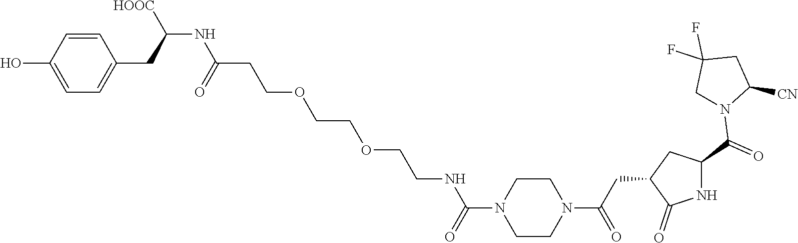 [SMILES: N#C[C@@H]1CC(F)(F)CN1C(=O)[C@@H]1C[C@@H](CC(=O)N2CCN(C(=O)NCCOCCOCCC(=O)N[C@@H](Cc3ccc(O)cc3)C(=O)O)CC2)C(=O)N1]

To a solution of 85 (10.7 mg, 0.027 mmol) in DCM (0.6 mL), DIPEA (0.014 mL, 0.081 mmol) was added under argon atmosphere. The reaction mixture was stirred at −78° C. for 10 min and solution of triphosgene (2.4 mg, 0.0089 mmol) prepared in DCM (0.5 mL) was added via syringe to the reaction mixture. The reaction mixture was allowed to warm up to rt over the period of 2.5 h and solution of 72 (11 mg, 0.027 mmol) prepared in DCM (0.5 mL) and DIPEA (0.014 mL, 0.081 mmol) The reaction progress was monitored by LC/MS. The reaction mixture was added to a stirred solution of 5% aq. Citric acid and aqueous layer was extracted with ethyl acetate. The combined organic layer was washed with water, brine and dried over sodium sulfate. The solvent was evaporated in vacuo and the crude was purified by column chromatography on silica gel (DCM/MeOH) to obtain, tert-butyl (3-(2-(2-(4-(2-((3S,5S)-5-((S)-2-cyano-4,4-difluoropyrrolidine-1-carbonyl)-2-oxopyrrolidin-3-yl)acetyl)piperazine-1-carboxamido)ethoxy)ethoxy)propanoyl)-L-tyrosinate 40 mg, which is dissolved in DCM (1 mL) and TFA (1 mL) was added slowly and stirred the content at rt for 1 h. The reaction was monitored by LC/MS and solvent was evaporated in vacuo and to the residue ether was added. The precipitated solid was filtered and washed with ether and dried under vacuum to obtain (3-(2-(2-(4-(2-((3S,5S)-5-((S)-2-cyano-4,4-difluoropyrrolidine-1-carbonyl)-2-oxopyrrolidin-3-yl)acetyl)piperazine-1-carboxamido)ethoxy)ethoxy)propanoyl)-L-tyrosine, 30 mg and purified by RP-HPLC.

### 4-(2-((E)-2-((E)-2-(4-((S)-13-carboxy-1-(4-(2-((3S,5S)-5-((S)-2-cyano-4,4-difluoropyrrolidine-1-carbonyl)-2-oxopyrrolidin-3-yl) acetyl) piperazin-1-yl)-1,11-dioxo-5,8-dioxa-2,12-diazatetradecan-14-yl) phenoxy)-3-(2-((E)-3,3-dimethyl-5-sulfo-1-(4-sulfobutyl) indolin-2-ylidene)ethylidene) cyclohex-1-en-1-yl) vinyl)-3,3-dimethyl-5-sulfo-3H-indol-1-ium-1-yl) butane-1-sulfonate

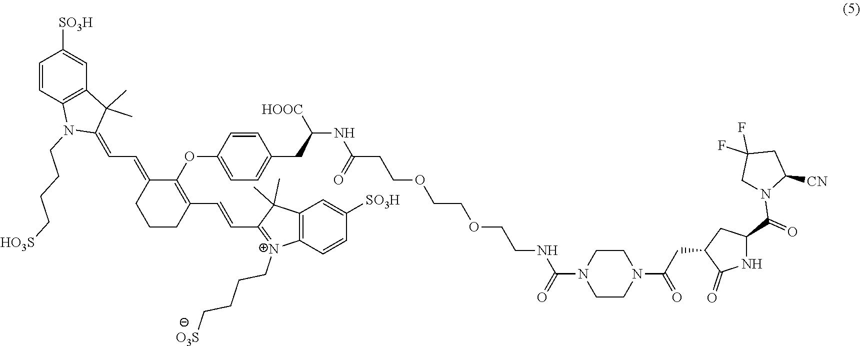

A 5 mL round bottom flask was charged with (3-(2-(2-(4-(2-((3S,5S)-5-((S)-2-cyano-4,4-difluoropyrrolidine-1-carbonyl)-2-oxopyrrolidin-3-yl)acetyl)piperazine-1-carboxamido) ethoxy) ethoxy) propanoyl)-L-tyrosine (5 mg, 0.007 mmol), Na₂CO₃ (7.2 mg, 0.07 mmol) and DMSO (0.6 mL) was added and stirred. S0456 (6.5 mg, 0.0.007 mmol) was added to this and the content was stirred at 60° C. oil bath for 20 h. The reaction was monitored by LC/MS, the reaction mixture was cooled to rt and purified by RP-HPLC to obtain the compound 5 in 23% yield.

### 4-(2-((E)-2-((E)-2-(4-((S)-1-(4-(2-((3S,5S)-5-((S)-2-carbamoyl-4,4-difluoropyrrolidine-1-carbonyl)-2-oxopyrrolidin-3-yl) acetyl) piperazin-1-yl)-13-carboxy-1,11-dioxo-5,8-dioxa-2,12-diazatetradecan-14-yl) phenoxy)-3-(2-((E)-3,3-dimethyl-5-sulfo-1-(4-sulfobutyl) indolin-2-ylidene)ethylidene) cyclohex-1-en-1-yl) vinyl)-3,3-dimethyl-5-sulfo-3H-indol-1-ium-1-yl) butane-1-sulfonate

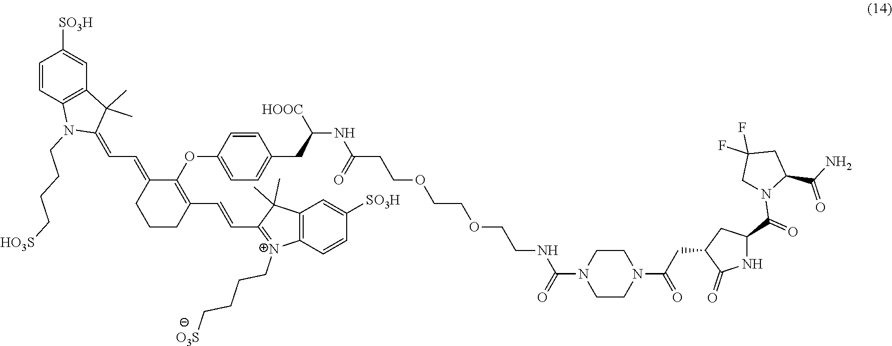

A 5 mL round bottom flask was charged with (3-(2-(2-(4-(2-((3S,5S)-5-((S)-2-cyano-4,4-difluoropyrrolidine-1-carbonyl)-2-oxopyrrolidin-3-yl)acetyl)piperazine-1-carboxamido) ethoxy) ethoxy) propanoyl)-L-tyrosine (1 mg, 0.0014 mmol), was dissolved in DMSO (0.6 mL) and water was added. The pH of the reaction mixture was adjusted to 10 using IN sodium carbonate solution and S0456 (1.2 mg, 0.0014 mmol) was added to this and the content was stirred at 65° C. oil bath for 3 h. The reaction was monitored by LC/MS, the reaction mixture was cooled to rt and purified by RP-HPLC to obtain the compound 14 in 41% yield.

### Example 3: In vivo studies

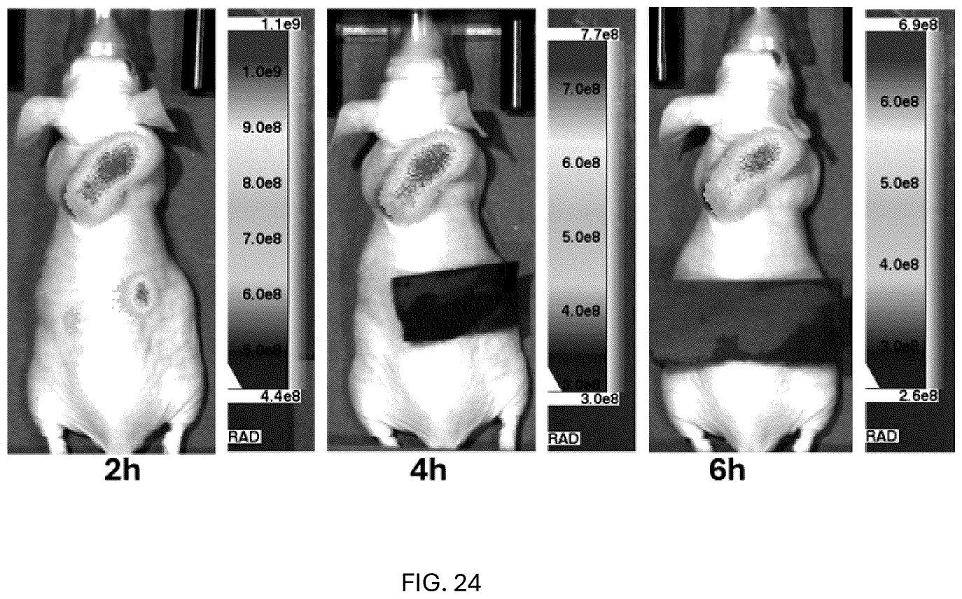

U87MG human Malignant Glioma tumor xenograft bearing mice were injected with 10 nmol of compound 3. The mice were euthanized and imaged with IVIS imager (ex=745 nm, em=ICG, exposure time=1 s) at different time intervals. (see FIG. 24)

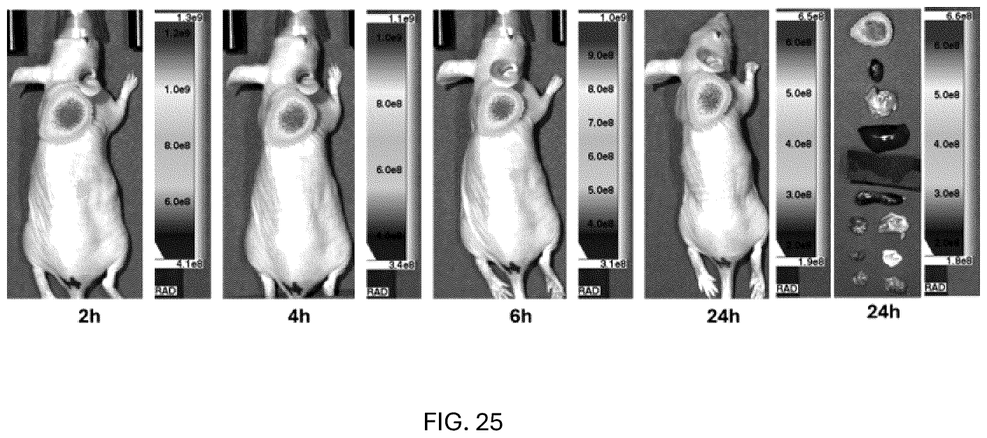

HT human colorectal tumor xenograft bearing mice were injected with 10 nmol of compound 5. The mice were euthanized and then imaged with IVIS imager (ex=745 nm, em=ICG, exposure time=1 s) at different time intervals. Selected tissues were also harvested and imaged (see FIG. 25).

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

It will be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound having a formula B—X—Z, wherein
B comprises a Fibroblast Activating Protein (FAP)-targeting molecule or FAP inhibitor (FAPI);
X comprises a spacer; and
Z comprises an imaging agent.

2. The compound of claim 1, wherein B is a monovalent group derived from a FAP-targeted molecule.

3. The compound of claim 1, wherein B is a small molecule comprising an extended hydrophobic moiety with alpha keto amide unit, a substituted alpha keto amide, or a substituted alpha keto amide with aromatic moiety with methoxy units.

4. The compound of claim 1, wherein B has a formula: 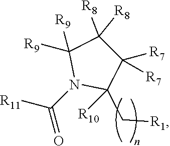
wherein R₁ is CHO, C═CH, CH(CN)₂, CN, NHCN, B(OH)R₂, COR₂, Zn(OH), PO(OH)R₂, SO₂R₂, Si(OH)₂R₂, a halogen, OH, H or COCONHR₃;
wherein R₂ is OH, SH, NH₂, O-alkyl, S-alkyl, NH-alkyl, a halogen, or H; and
R₃ is H, a halogen, OH, methyl, ethyl, isopropyl, tBu, cyclopropyl, alkyl, alkyl-OH, CN, CHPh₂, adamantane or 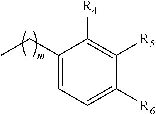
wherein R₄, R₅, or R₆ are independently selected from the group consisting of H, OCH₃, SCH₃, COR₂, SO₂R₂, PO(OH)R₂, Si(OH)₂R₂, halogen, OH, CN, B(OH)R₂, and combinations thereof;
R₇, R₈, R₉, or R₁₀ are independently selected from the group consisting of H, a halogen, OH, OCH₃, SCH₃, CN, COR₂, SO₂R₂, PO(OH)R₂, Si(OH)₂R₂, B(OH)R₂, a C₁-C₁₀ alkyl, and combinations thereof;
n and m are independently selected from 0, 1, 2, 3, 4, or 5;
R₁₁ is selected from the group consisting of 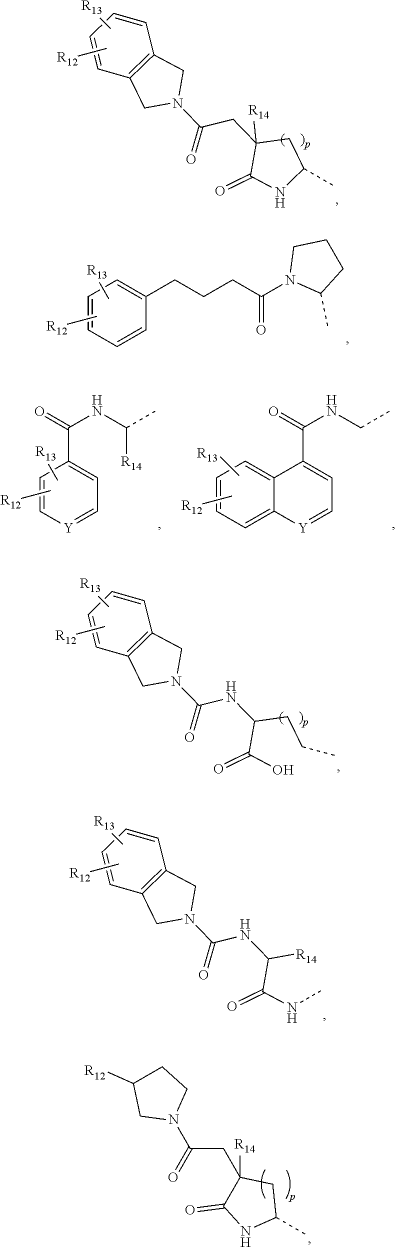 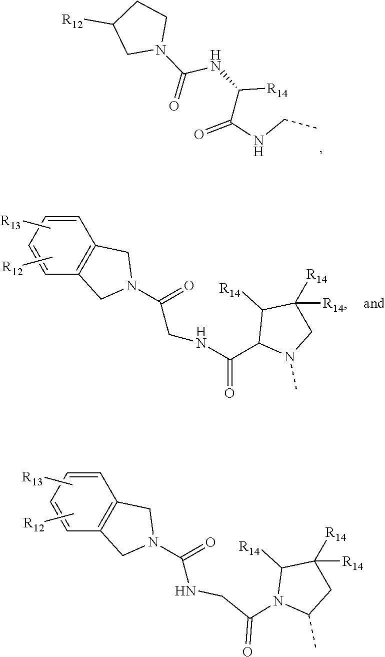
wherein R₁₂ is (CH₂)q-X, X=COOH, SH, OH, NH₂, halogen, or maleimide;
R₁₃ is H, halogen, Me, OH, SH, OCH₃, OBn, or CH₂OH;
R₁₄ is H, F, CH₃, OCH₃, Isopropyl, or Tert-butyl;
Y is CH or N; and
p and q are independently selected from 0, 1, 2, 3, 4, or 5.

5. The compound of claim 1, wherein X is selected from the group consisting of six aminohectanoic acid (SAHA), eight aminooctonoic acid (EAOA), polyethylene glycol (PEG), polyethylene amine (PEA) unit, and N-amino-dPEG₂ acid.

6. The compound of claim 1, wherein X is a peptide comprising at least one aryl group or at least one aryl alkyl group, each of which is optionally substituted.

7. The compound of claim 6, wherein the peptide comprises at least two aryl or aryl alkyl group, wherein a first aryl or aryl alkyl group is about 6 atoms to about 15 atoms.

8. The compound of claim 6, wherein the peptide compromises at least one amino acid selected from the group consisting of a quaternary amine containing amino acid, an acidic amino acid, a basic amino acid, a neutral polar amino acid, a neutral nonpolar amino acid, an aromatic amino acid, and amino acid derivatives.

9. The compound of claim 8, wherein the acidic amino acid is selected from the group consisting of aspartic acid and glutamic acid; and/or
the basic amino acid is selected from the group consisting of arginine, lysine, histidine, and ornithine; and/or
the neutral polar amino acid is selected from the group consisting of glycine, serine, threonine, cysteine, tyrosine, asparagine, and glutamine; and/or
the neutral nonpolar amino acid is selected from the group consisting of alanine, leucine, isoleucine, valine, proline, phenylalanine, tryptophan, and methionine;
and/or amino acid derivative is derived from tyrosine.

10. The compound of claim 8, wherein the amino acid derivative is selected from the group consisting of:

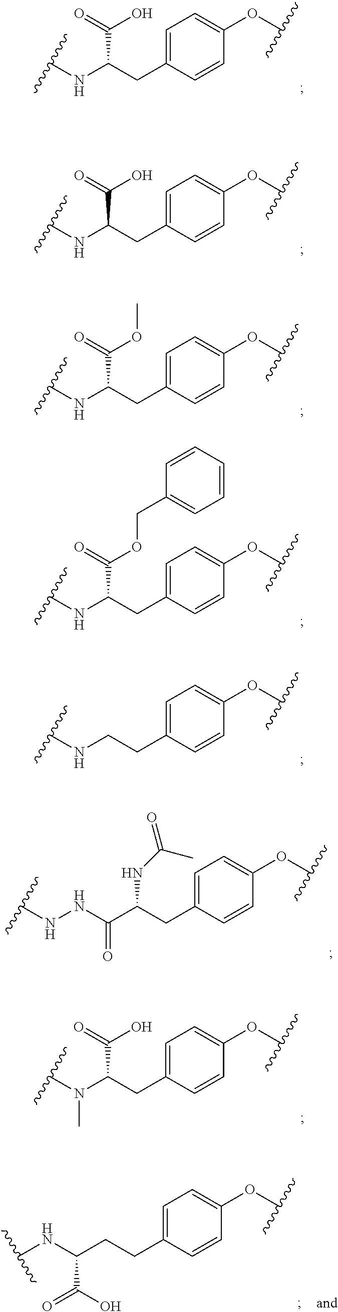

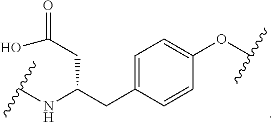 [SMILES: *N[C@H](CC(=O)O)Cc1ccc(O*)cc1]

11. The compound of claim 1, wherein X comprises an amino acid spacer with sulfur-containing side chain group, wherein the sulfur-containing side group is selected from the group consisting of cysteine, methionine and a molecule containing a thiophenol moiety or an amino acid spacer with a chalcogen-containing side chain group.

12. The compound of claim 1, wherein X comprises a single amino acid selected from the group consisting of tyrosine, cysteine, lysine, tyramine, a tyrosine derivative, a cysteine derivative, and a lysine derivative.

13. The compound of claim 1, wherein X comprises an amino acid isotope.

14. The compound of claim 1, wherein X has a length of about 1 atoms to about 20 atoms.

15. The compound of claim 1, wherein Z comprises a monovalent group derived from a NIR dye.

16. The compound of claim 15, wherein the NIR dye is selected from the group consisting of: 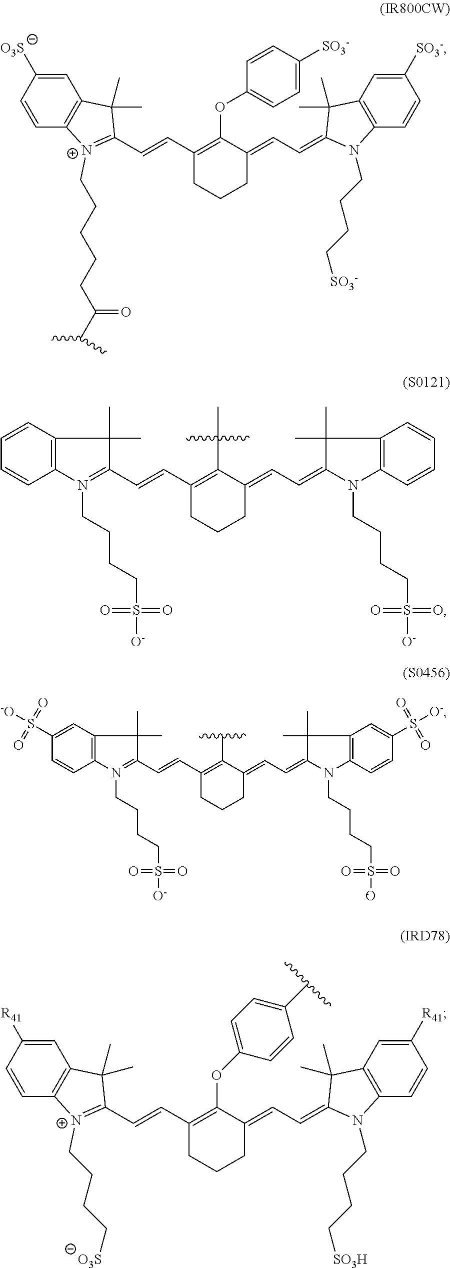 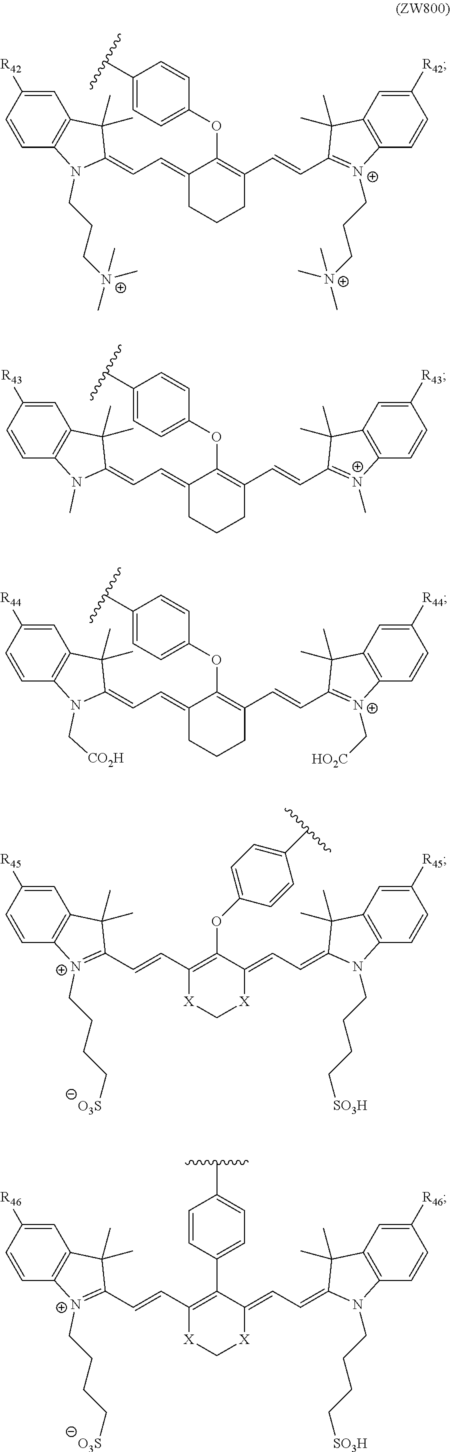 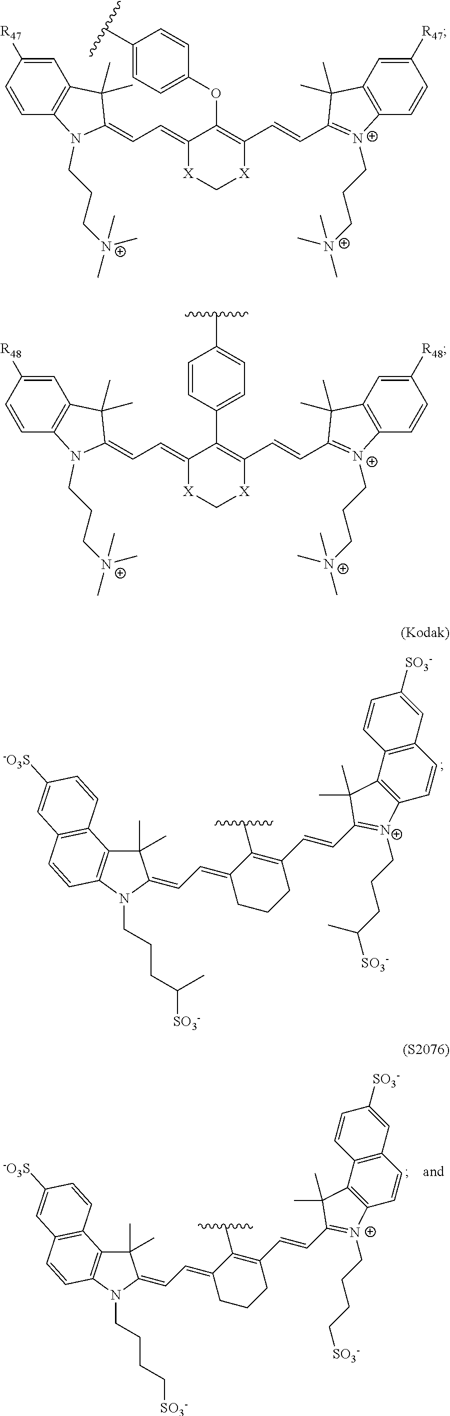 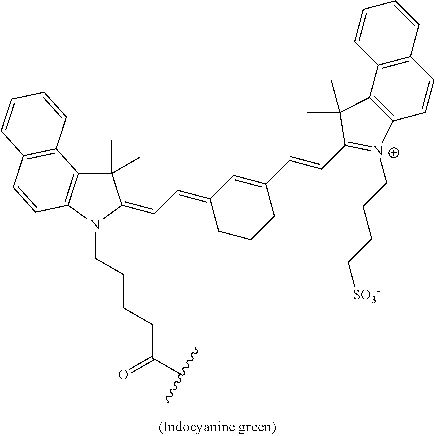
wherein R41, R42, R43, R44, R45, R46, R47, R48=H or SO₃H; X═O, S, or N.

17. A compound having the following formula: 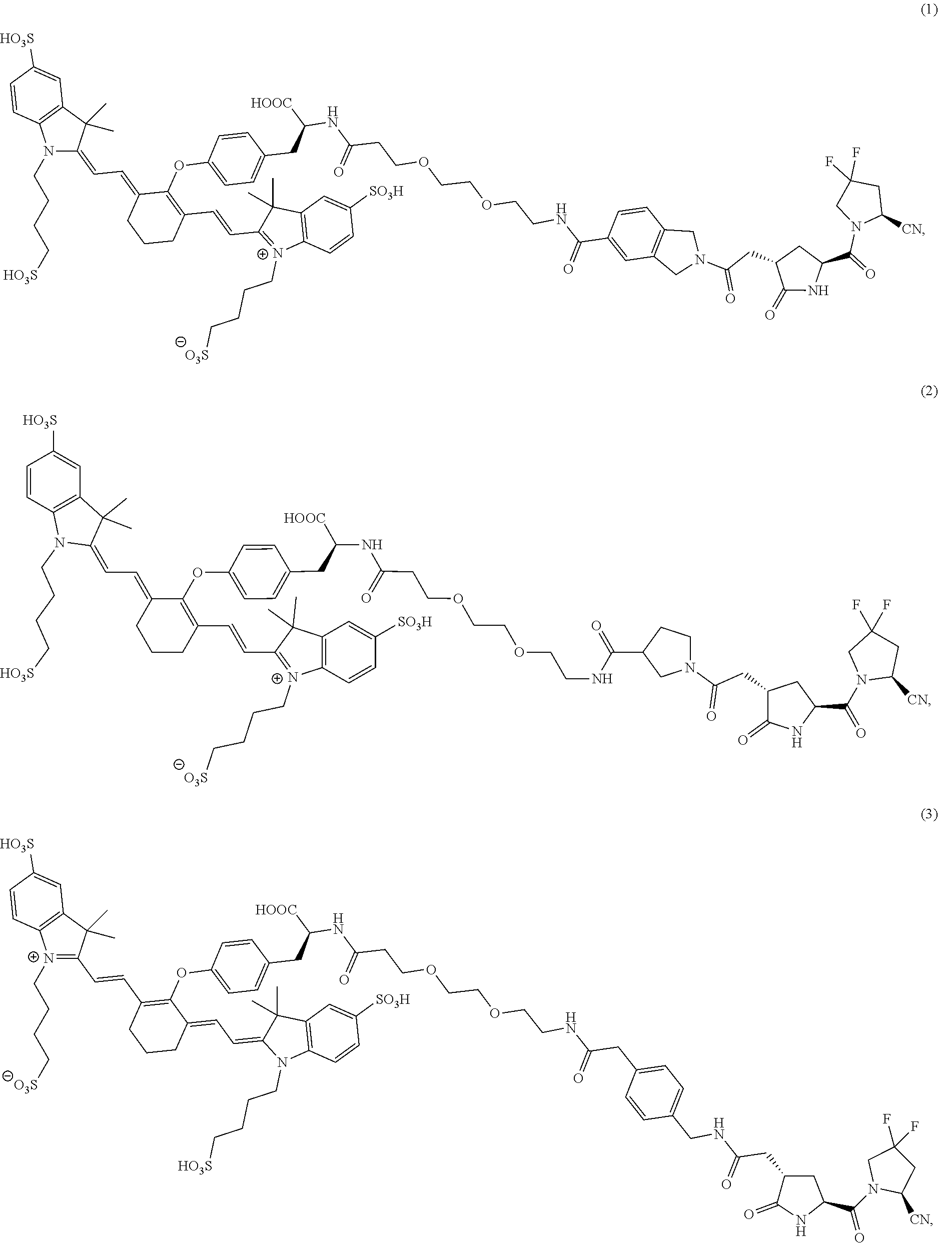 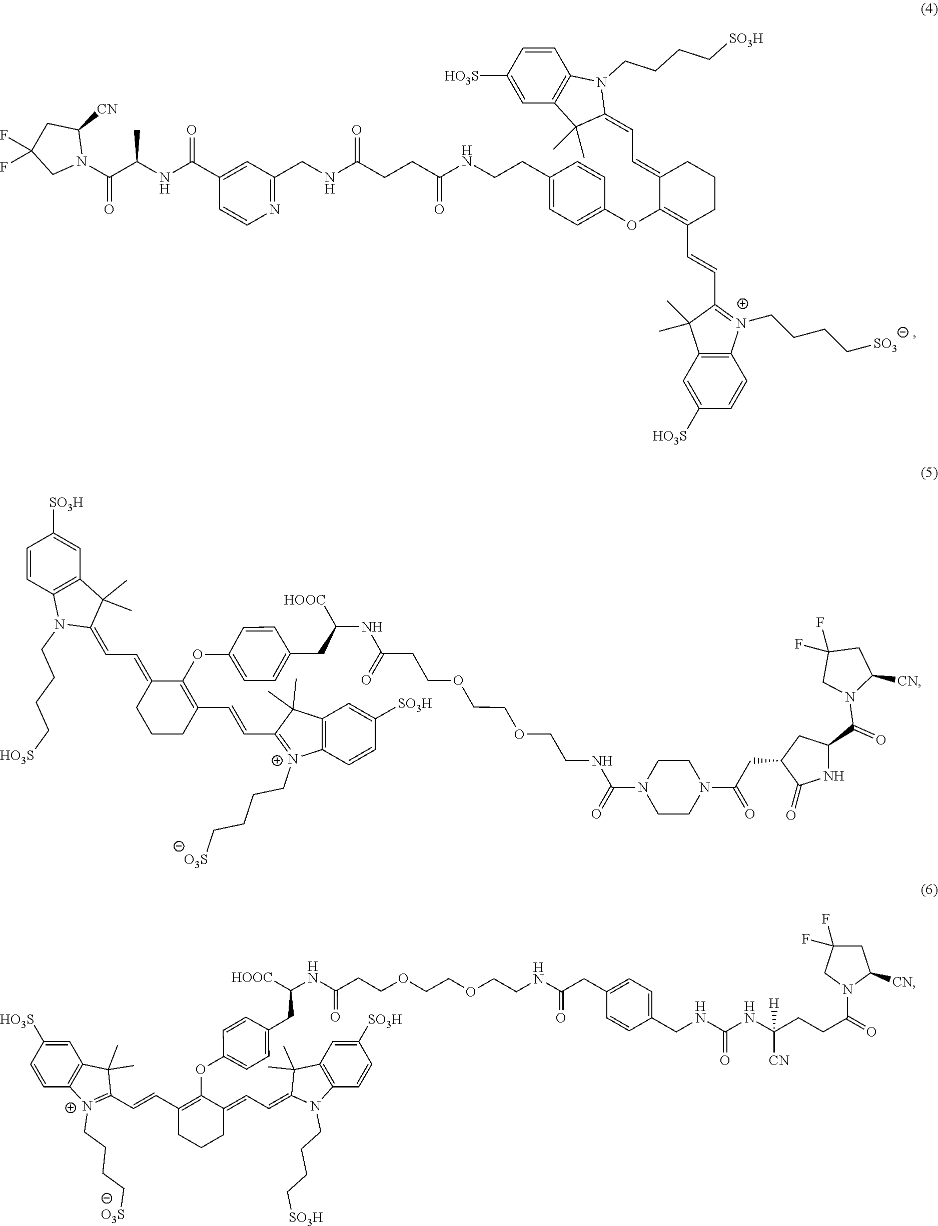 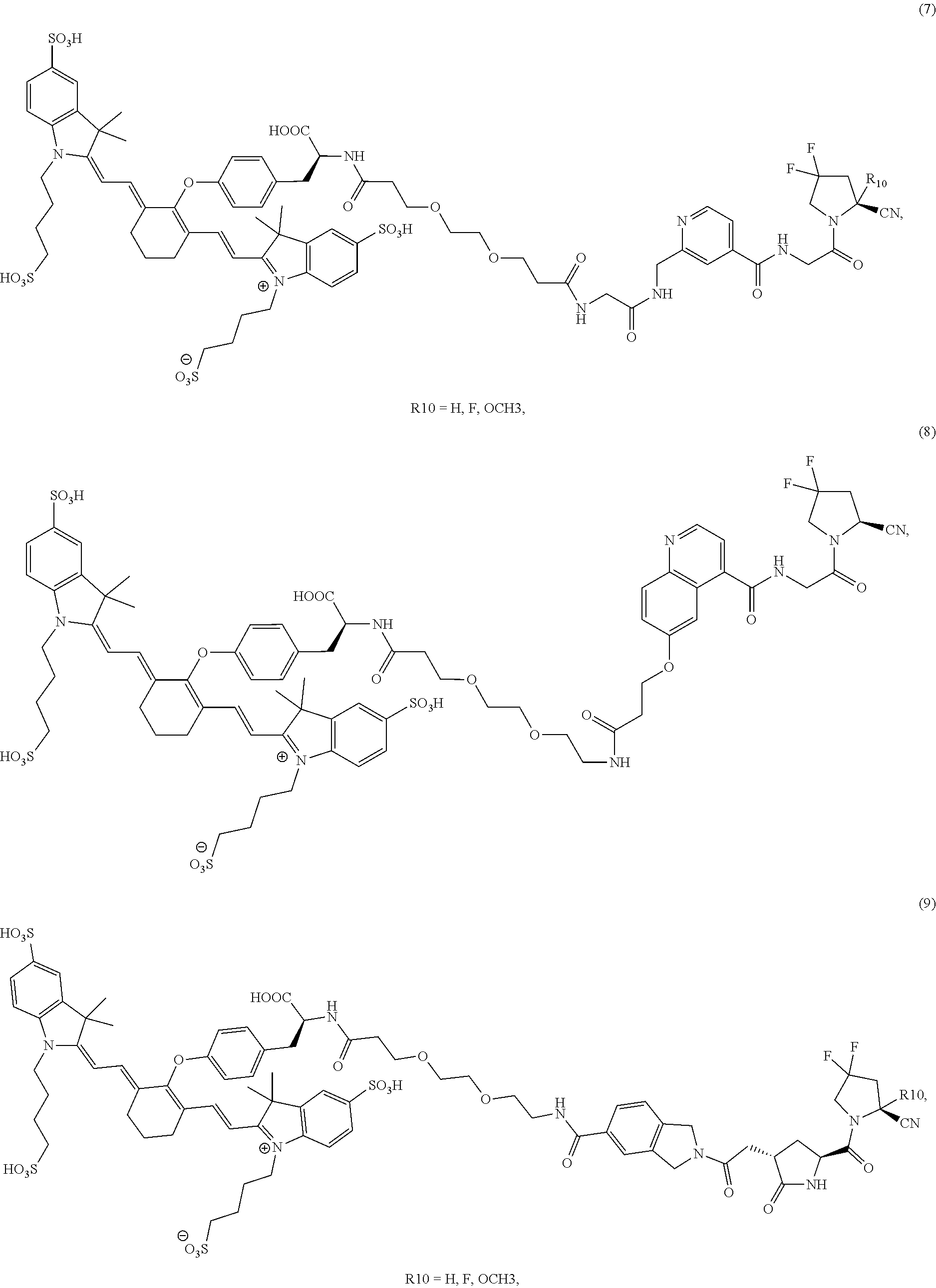 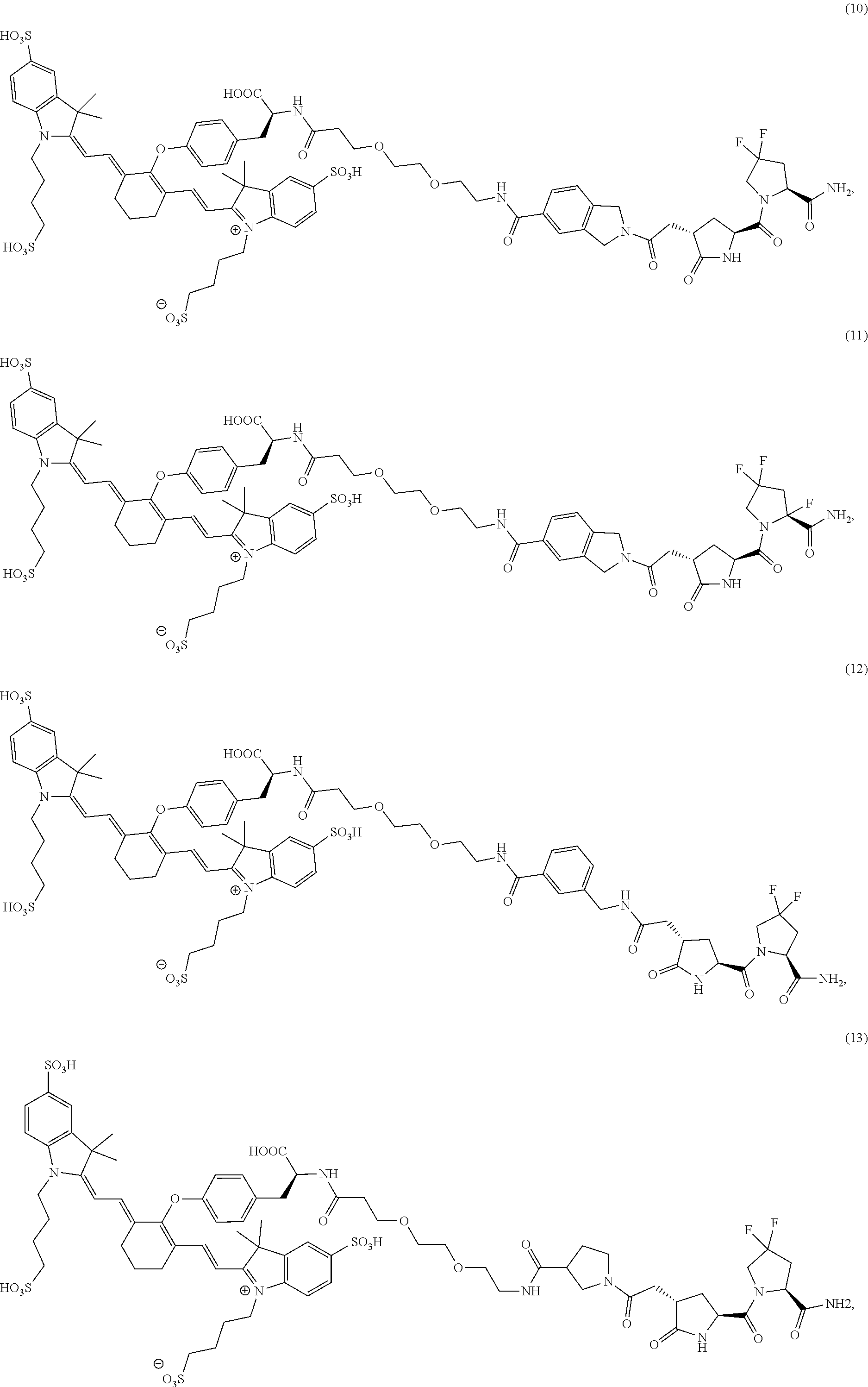 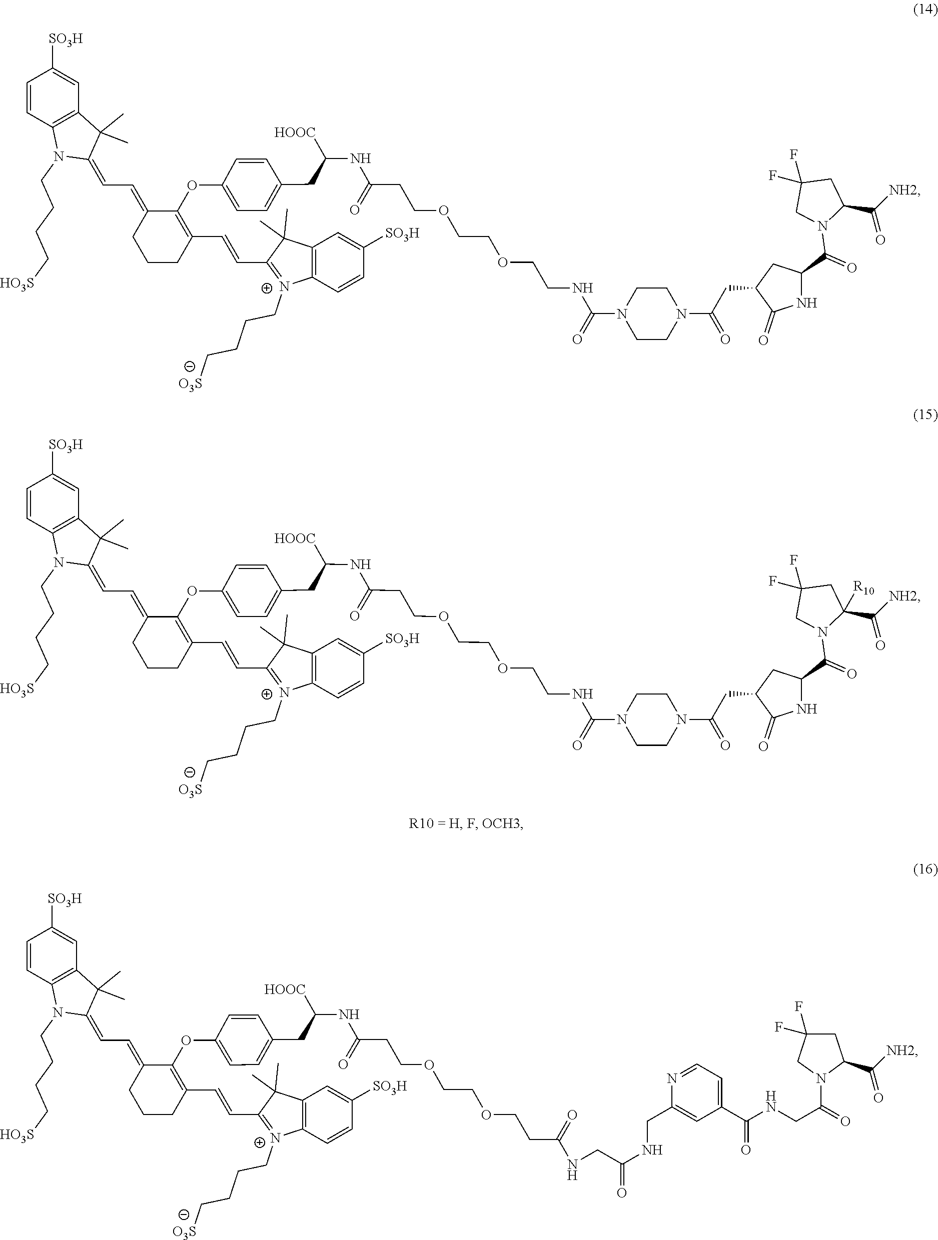 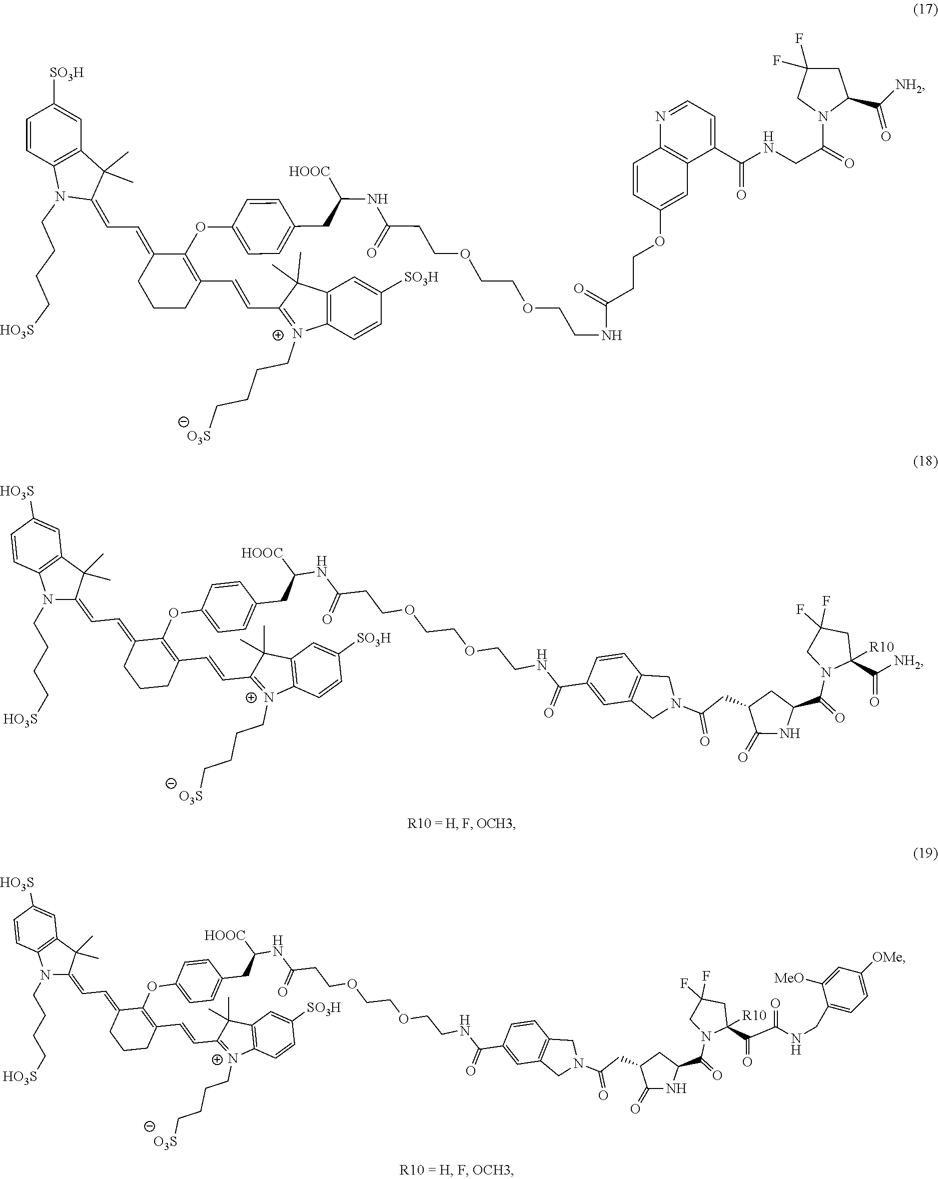 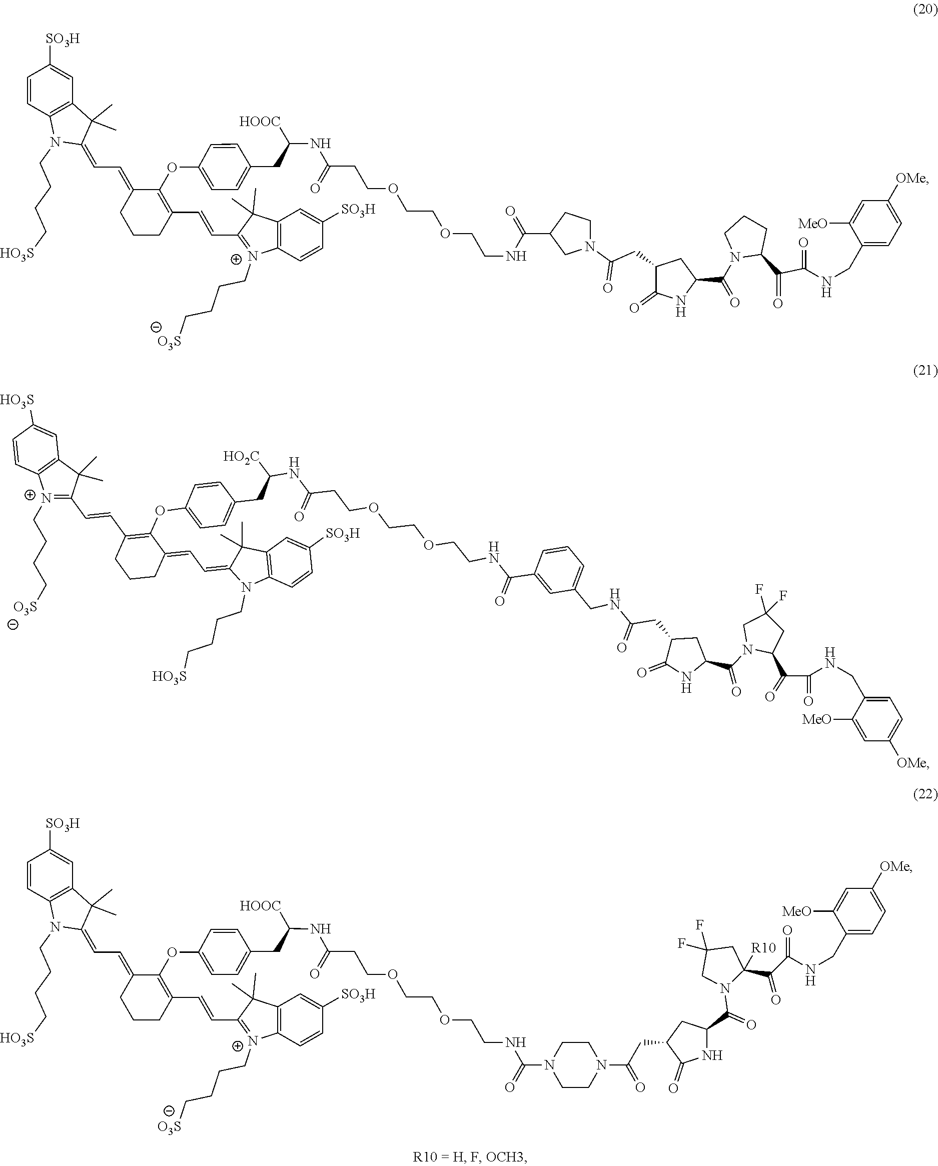 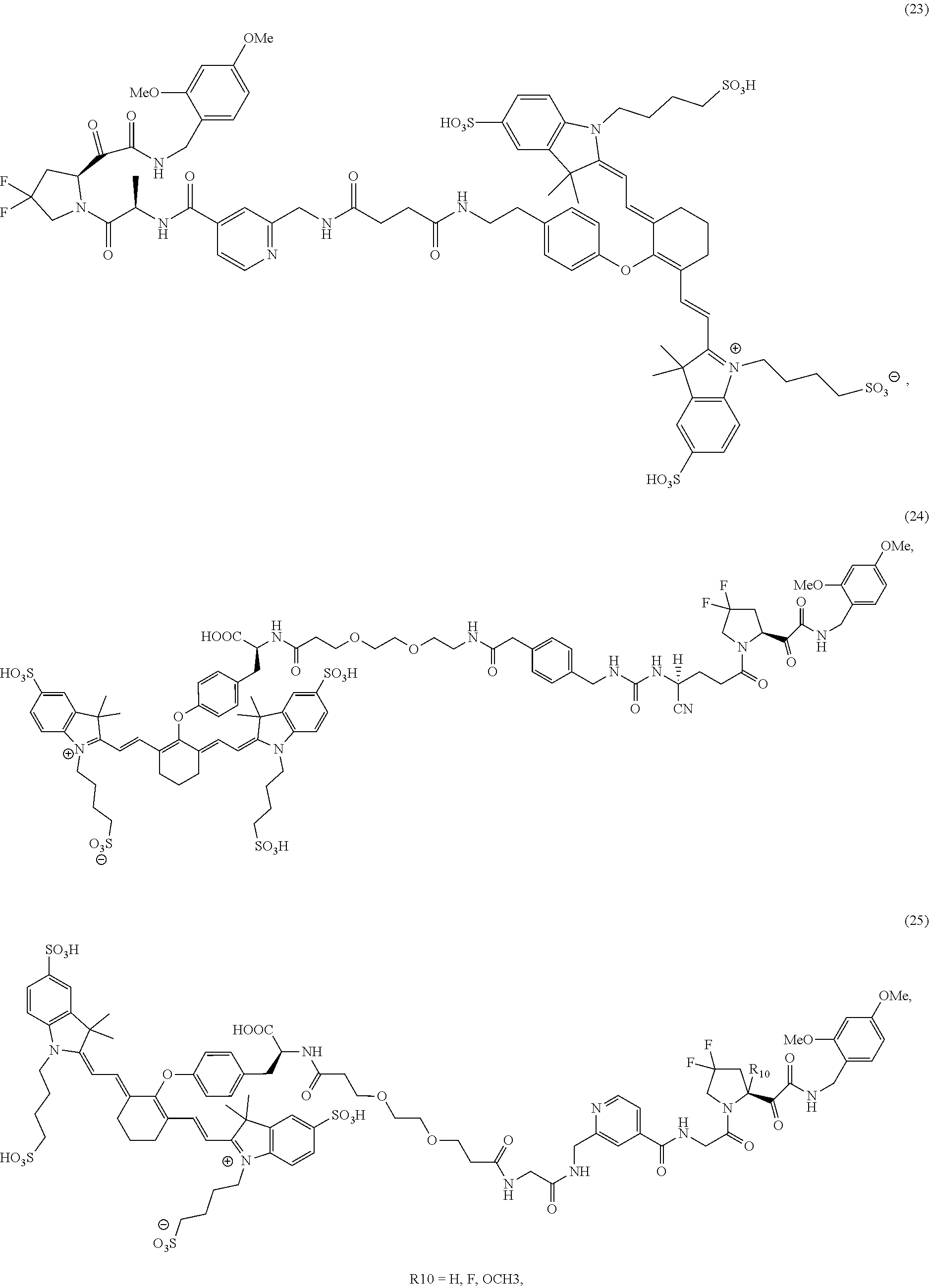 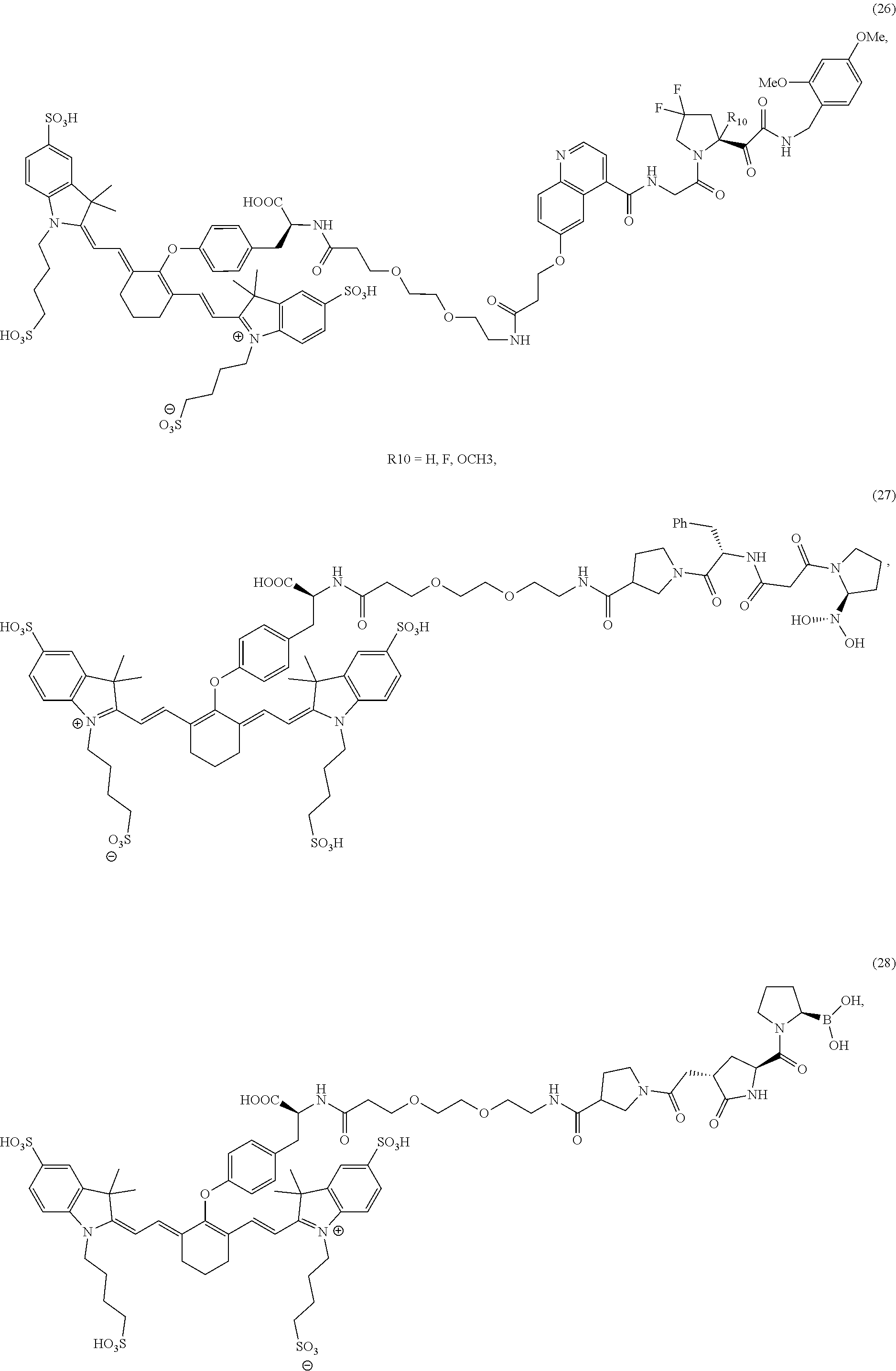 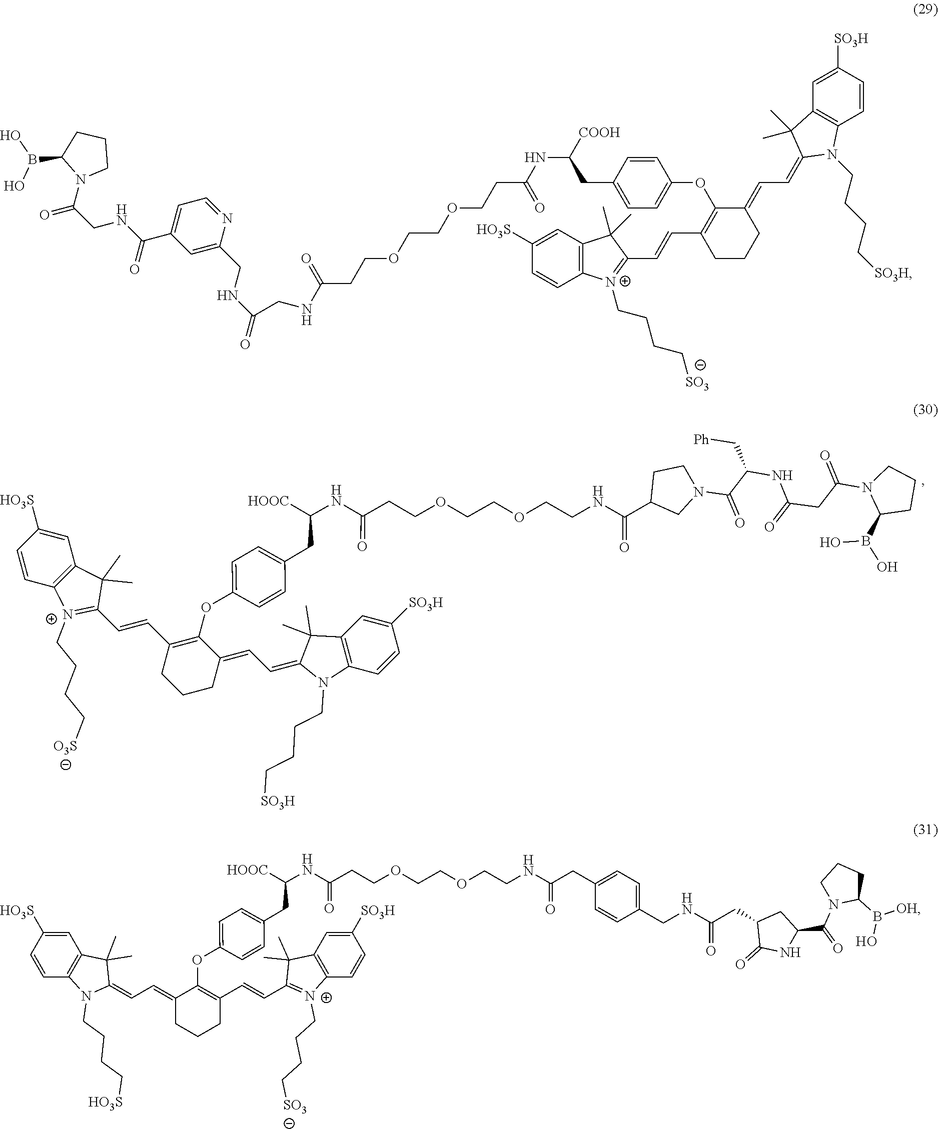 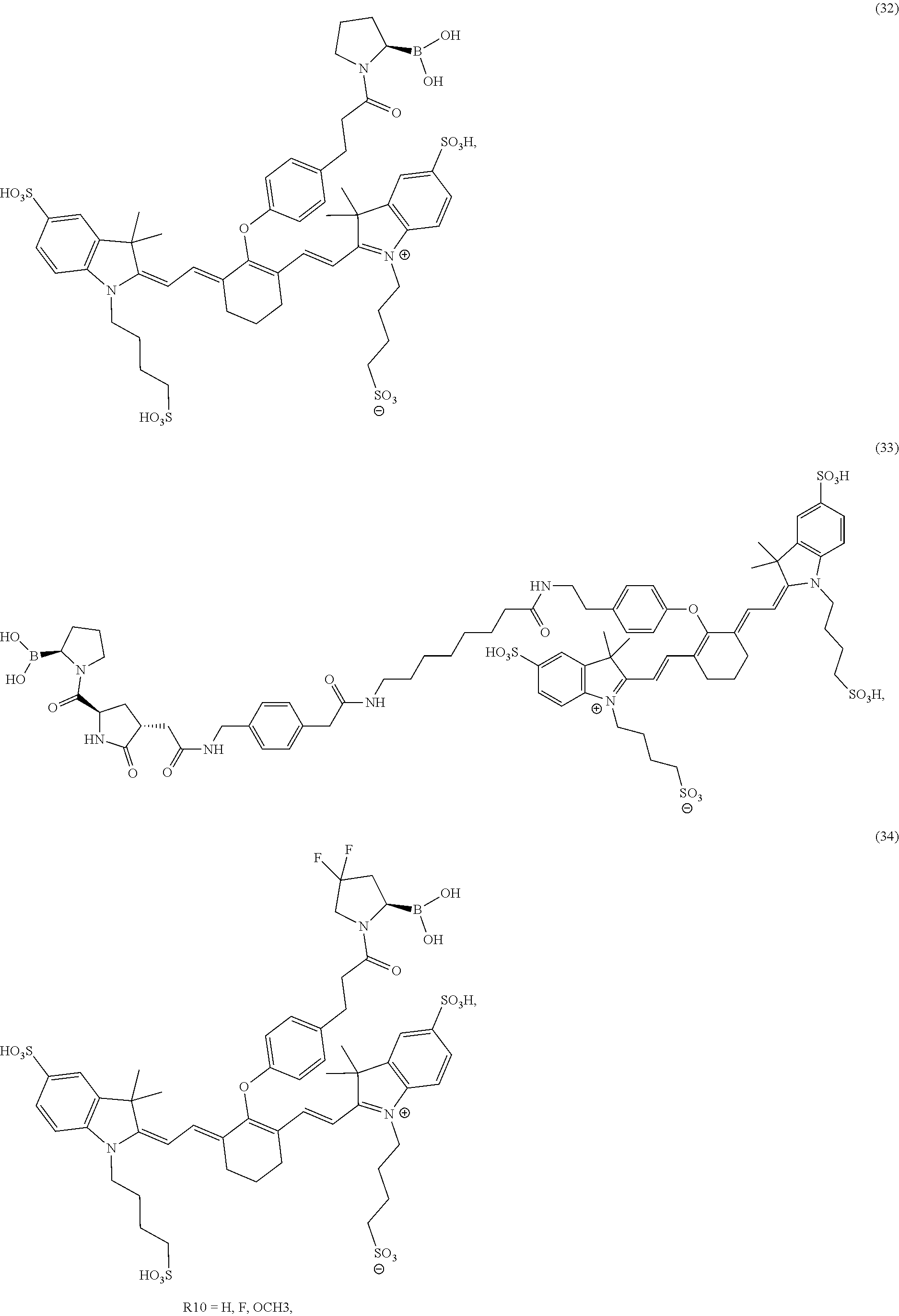 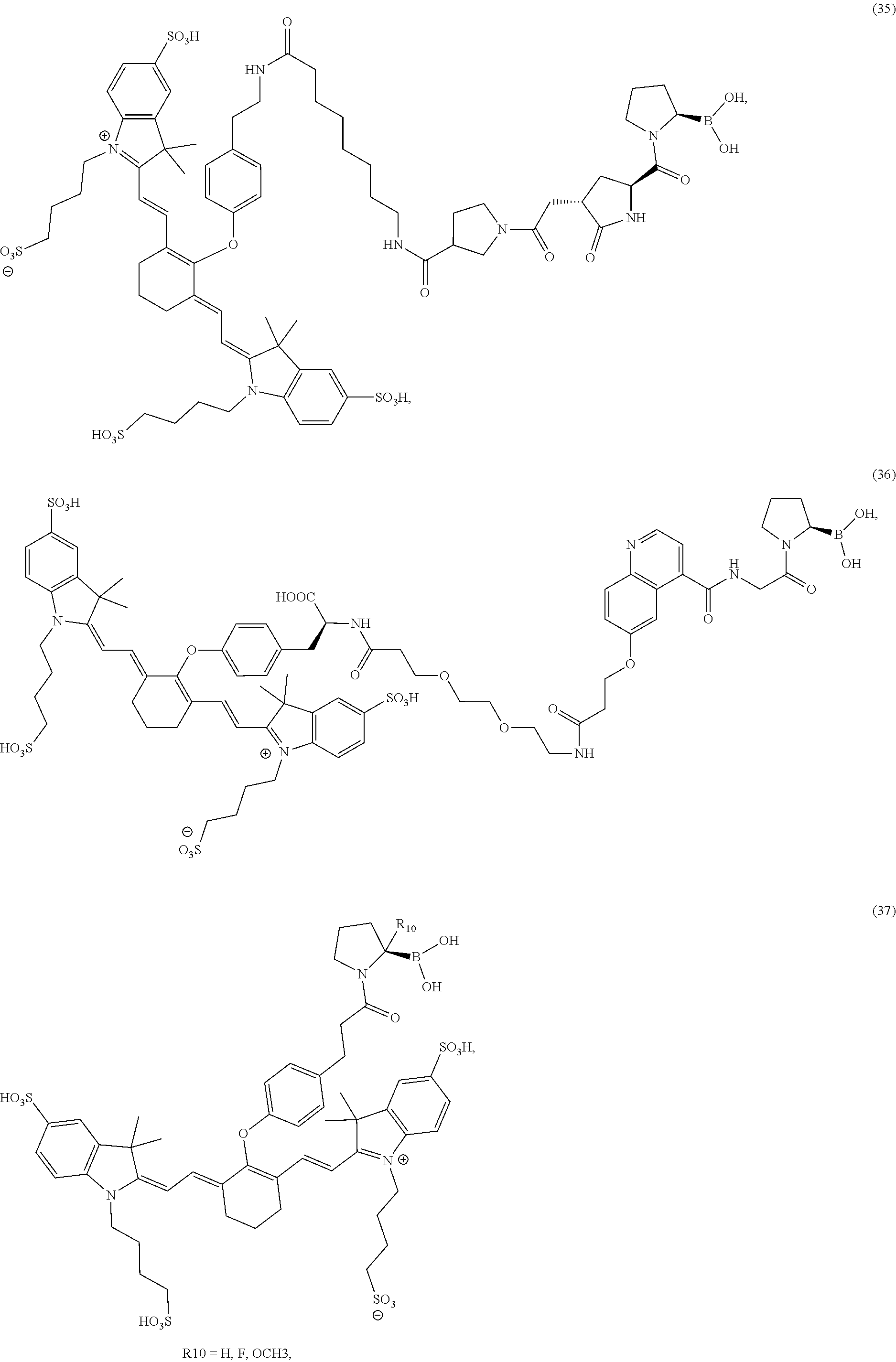 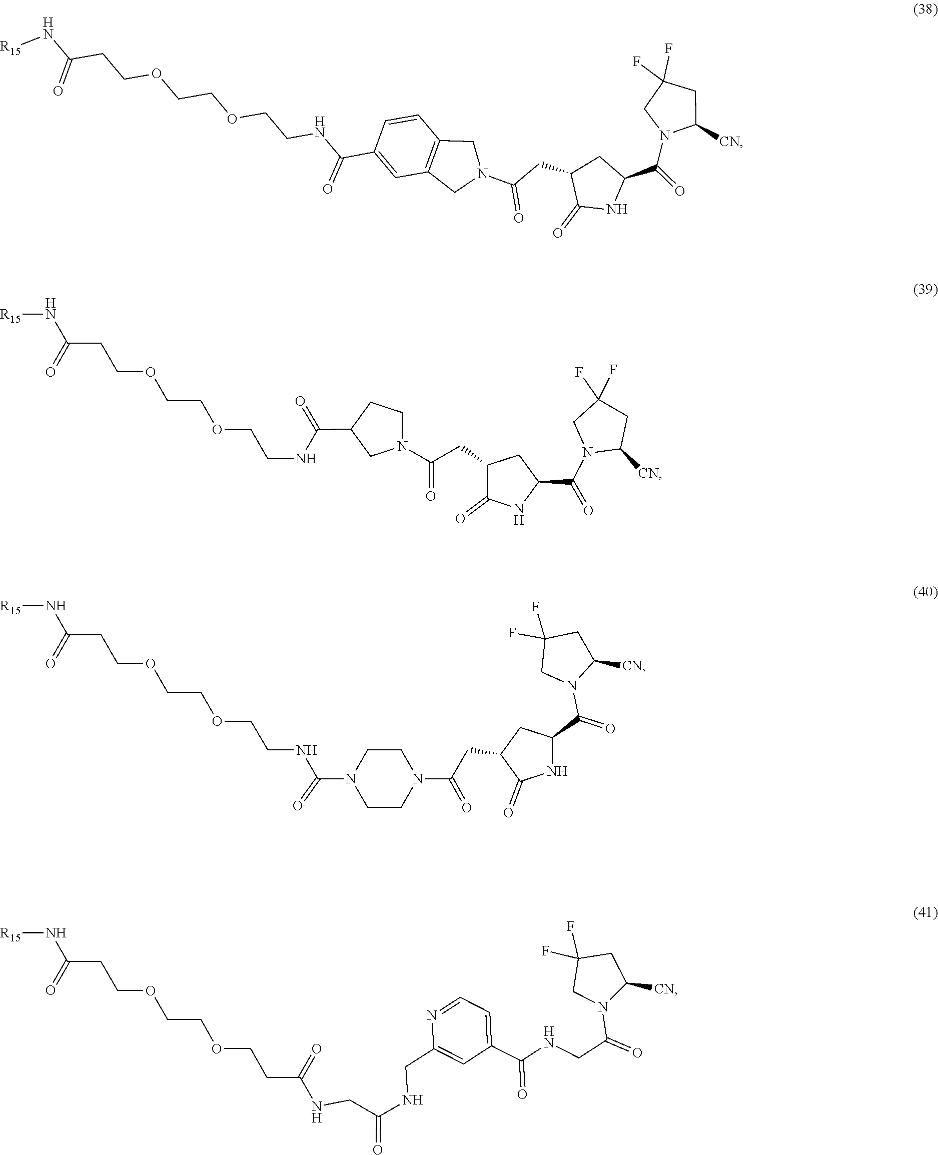 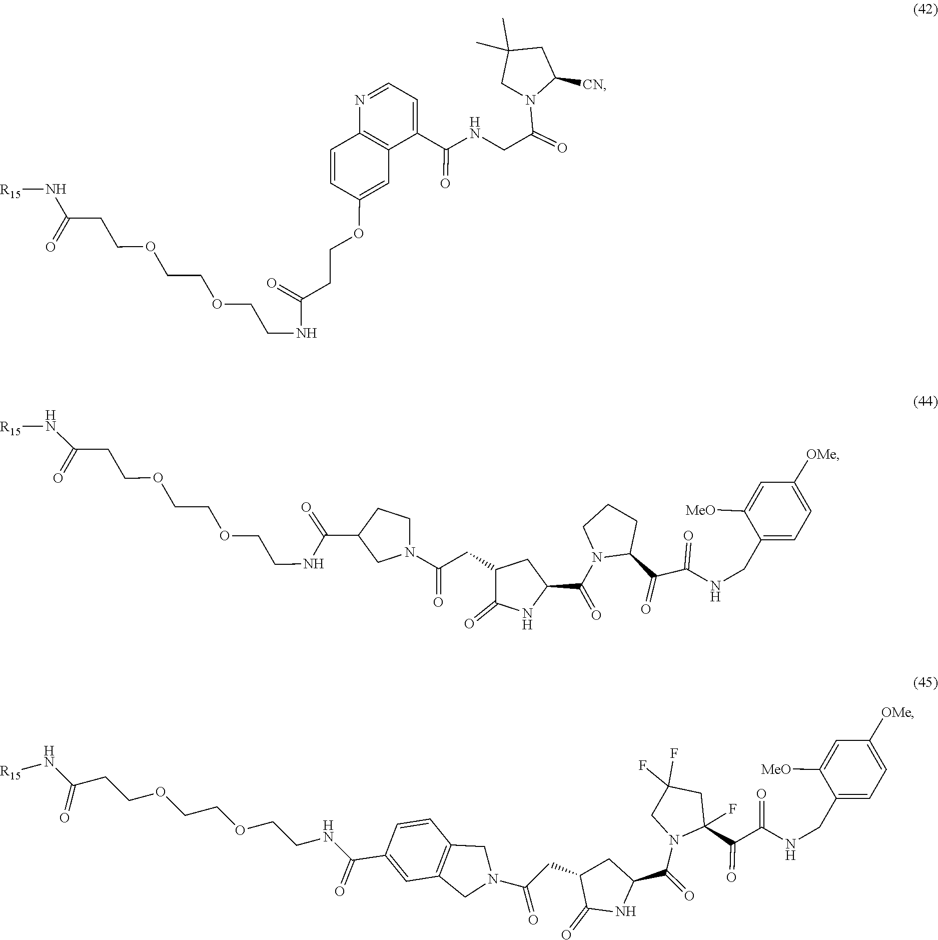 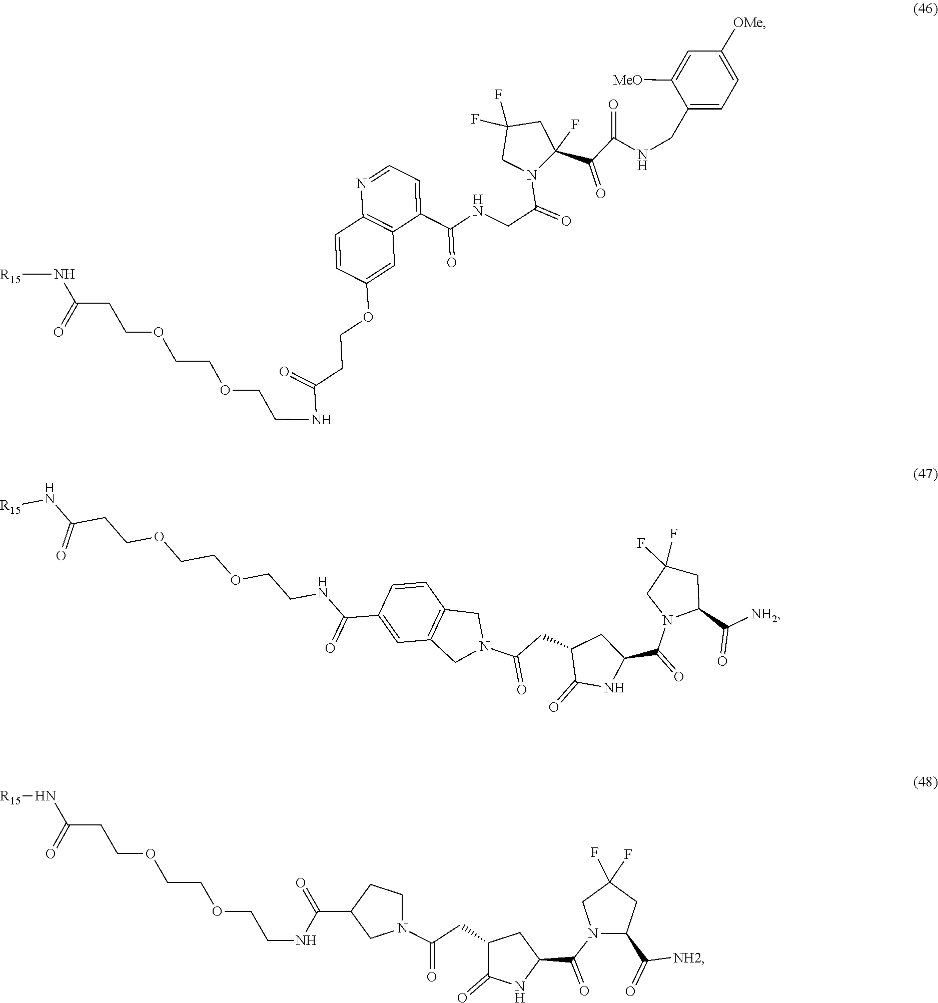 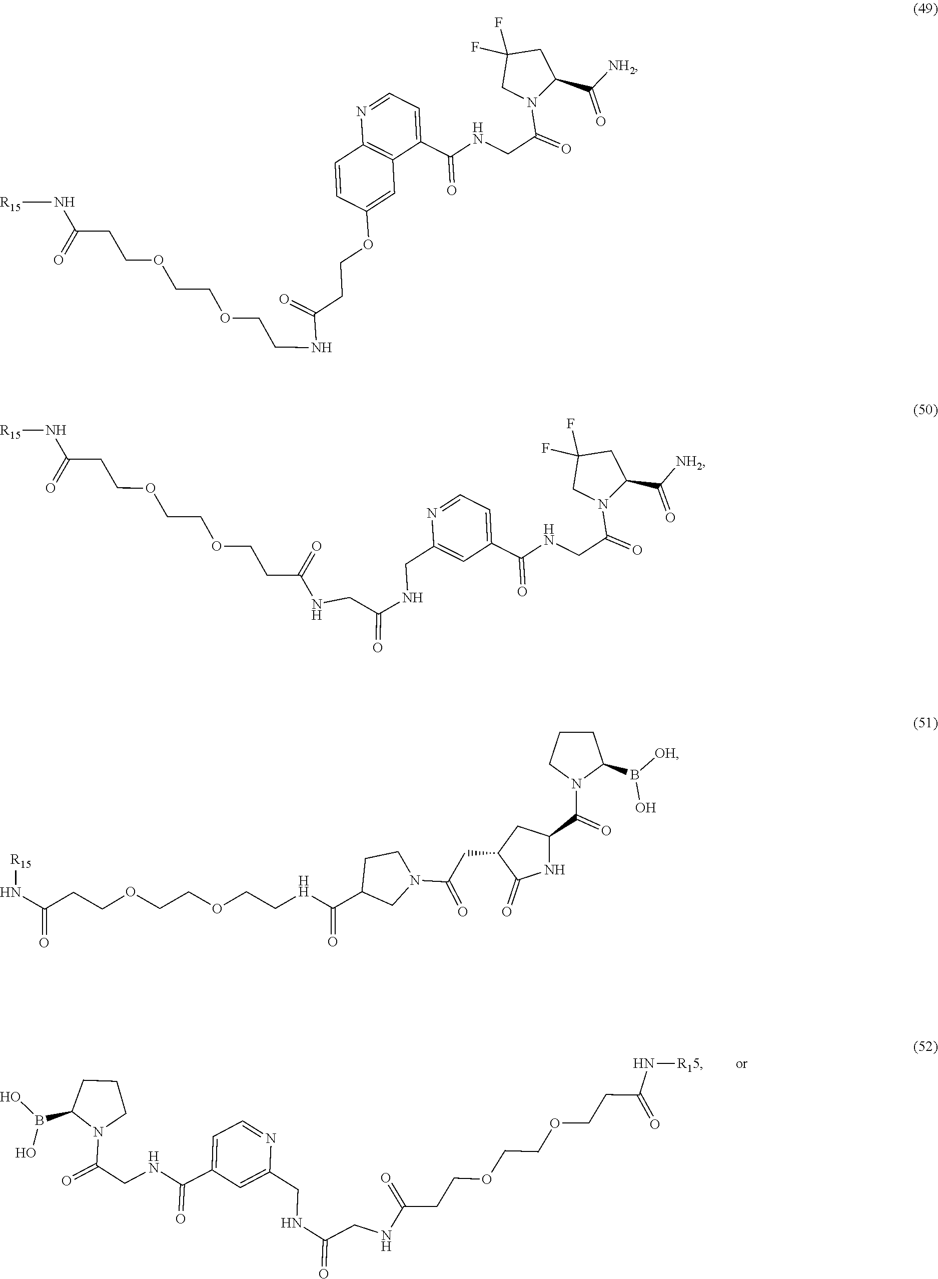 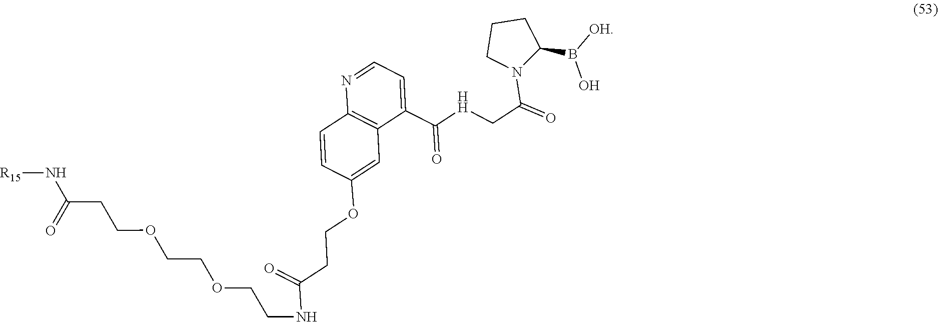
wherein R15 is chelating group selected from the group consisting of: 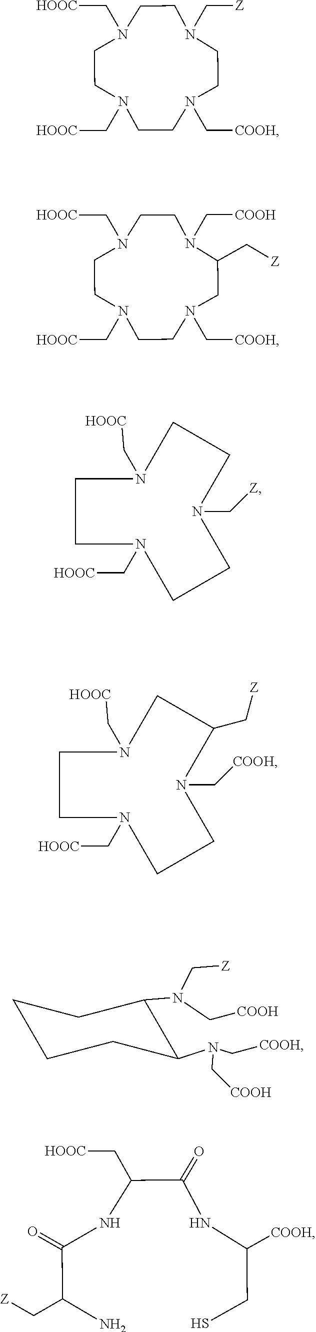 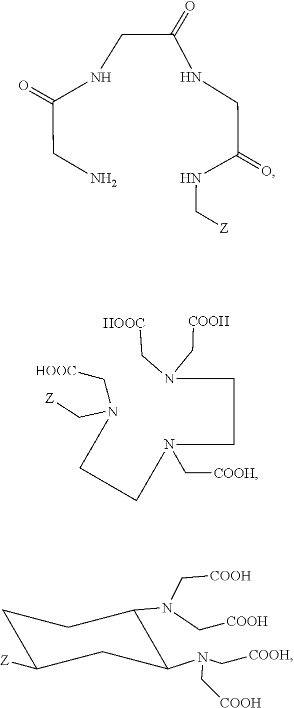
and Z=CO—, CH₂-Ph-NH—CO—, CH (COOH)—(CH₂)₂—CO—.
